(19) United States
(12) Patent Application Publication
(10) Pub. No.: US 2025/0073324 A1
(43) Pub. Date: Mar. 6, 2025

(54) MODIFIED ARENAVIRUS PARTICLES EXPRESSING MUTANT KRAS, MUTATED CANCER DRIVER GENE, OR TUMOR-ASSOCIATED ANTIGEN AS CANCER IMMUNOTHERAPIES

(71) Applicant: Hookipa Biotech GmbH, Vienna (AT)

(72) Inventors: Henning LAUTERBACH, Herrsching am Ammersee (DE); Jörg Christoph LAMPERT, Den Haag (NL); Mohamed HABBEDDINE, Basel (CH); Josipa RAGUZ, Vienna (AT); Timo SCHIPPERS, Vienna (AT); Sarah SCHMIDT, Vienna (AT); Sarah AHMADI-ERBER, Sallingberg (AT); Sandra ROSSKOPF, Deutsch-Wagram (AT); Klaus ORLINGER, Vienna (AT); Igor MATUSHANSKY, Brooklyn, NY (US)

(21) Appl. No.: 18/708,090

(22) PCT Filed: Nov. 7, 2022
(86) PCT No.: PCT/EP2022/081018

§ 371 Date: May 7, 2024

Related U.S. Application Data
(60) Provisional application No. 63/404,068, filed on Sep. 6, 2022.
(60) Provisional application No. 63/404,008, filed on Sep. 6, 2022.
(60) Provisional application No. 63/277,049, filed on Nov. 8, 2021.
(60) Provisional application No. 63/277,052, filed on Nov. 8, 2021.

(51) Int. Cl. A61K 39/00; C12N 7/00; C12N 9/14; C12N 15/86

(52) U.S. Cl. CPC A61K 39/001164; C12N 7/00; C12N 9/14; C12N 15/86; C12Y 306/05002; A61K 2039/5258; A61K 2039/575; C12N 2760/10022; C12N 2760/10023; C12N 2760/10034; C12N 2760/10043; C12N 2760/10052; C12N 2760/10062

(57) ABSTRACT

The invention relates to genetically modified arenaviruses suitable for the treatment of neoplastic diseases, such as cancer. The arenaviruses described herein may be suitable for treatment of neoplastic diseases and/or for the use in immunotherapies. In particular, provided herein are methods and compositions for treating a neoplastic disease by administering a genetically modified arenavirus, wherein the arenavirus has been engineered to include a nucleotide sequence encoding one or more antigenic fragment(s) of mutant KRAS alone or to further include a nucleotide sequence encoding one or more antigenic fragment(s) of a mutated cancer driver gene (e.g., a mutant TP53) or a tumor-associated antigen.

### CROSS-REFERENCE TO RELATED APPLICATIONS

This application claims priority to U.S. Provisional Application No. 63/404,008 filed Sep. 6, 2022, U.S. Provisional Application No. 63/404,068 filed Sep. 6, 2022, U.S. Provisional Application No. 63/277,049 filed Nov. 8, 2021, and U.S. Provisional Application No. 63/277,052 filed Nov. 8, 2021, the content of each of which is incorporated by reference in its entirety herein, and to which priority is claimed.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “13194-082-228_SequenceListing.xml”, was created on Nov. 4, 2022, and is 93,515 bytes in size.

### 1. INTRODUCTION

The invention relates to genetically modified arenaviruses suitable for the treatment of neoplastic diseases, such as cancer. The arenaviruses described herein may be suitable for treatment of neoplastic diseases and/or for the use in immunotherapies. In particular, provided herein are methods and compositions for treating a neoplastic disease by administering a genetically modified arenavirus, wherein the arenavirus has been engineered to include a nucleotide sequence encoding an or several antigenic fragment(s) of mutant KRAS alone or to further include a nucleotide sequence encoding an or several antigenic fragment(s) of one or several different mutated cancer driver gene(s) (e.g., a mutant TP53) or tumor-associated antigen(s).

### 2. BACKGROUND

There is an unmet medical need for the treatment of neoplastic diseases, such as cancer. The emerging field of immunotherapies holds promise for the treatment of these life-threatening diseases. In addition, combination therapies are being explored. However, as more therapies become available, the possible combinations are complex.

One strategy for immunotherapies involves arenavirus-based expression of mutant KRAS antigens, mutated cancer driver genes (e.g., a mutant TP53), tumor-associated antigens. See for example, WO2009/083210; WO/2016/075250; WO2017/198726; and WO2021/089853. Intratumoral administration of these immunotherapies has been described. See for example, WO2018/185307.

### 3. SUMMARY OF THE INVENTION

The present application relates to genetically modified arenaviruses suitable for the treatment of neoplastic diseases, such as cancer. In particular, the present application relates to an arenavirus particle, wherein
a. the arenavirus particle comprises an arenavirus genome comprising a nucleotide sequence encoding an antigenic fragment of mutant KRAS, wherein the antigenic fragment comprises the mutation; and
b. at least one arenavirus open reading frame (“ORF”) of the arenavirus genome is either (i) functionally inactivated or deleted; or (ii) located in a position other than the wild-type position of said at least one arenavirus ORF; or (iii) sequestered into two or more functional fragments and a fragment of the at least one arenavirus ORF is located in a position other than the wild-type position of said at least one arenavirus ORF.

The arenavirus particle, wherein the mutation in KRAS is at amino acid position G12, G13, A18, A59, Q61, K117, A146, or D119 of KRAS.

The arenavirus particle, wherein the mutation in KRAS is A18D, A59E, A59G, A59P, A59T, A59S, A59V, A146P, A146S, A146T, A146V, D119N, G12A, G12C, G12D, G12F, G12L, G12R, G12S, G12V, G13A, G13C, G13D, G13E, G13R, G13S, G13V, K117N, Q61E, Q61H, Q61K, Q61L, Q61P, Q61R or a combination thereof.

The arenavirus particle, wherein the mutation in KRAS is G12A, G12C, G12D, G12R, G12S, G12V, G13D, Q61H, Q61R, A146T or a combination thereof.

The arenavirus particle, wherein the mutation in KRAS is G13D, G12V, G12C, G12D, G12R or a combination thereof. In a more specific embodiment, the arenavirus genome comprises a nucleotide sequence encoding from N- to C-terminus fragments of mutant KRAS comprising the mutations G13D, G12V, G12C, G12D, and G12R, respectively. In other more specific embodiments, the arenavirus genome comprises a nucleotide sequence encoding fragments of mutant KRAS comprising the mutations G13D, G12V, G12C, G12D, and G12R in any possible order.

The arenavirus particle, wherein the arenavirus genome comprises a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of SEQ ID NO:20.

The arenavirus particle, wherein the arenavirus genome comprises a nucleotide sequence encoding an expression product whose amino acid sequence is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:19.

The arenavirus particle, wherein the fragment of mutant KRAS is 8, 9, 10, 11, 12, 13, 14, 15, 16, 17, 18, 19, 20, 21, 22, 23, 24, 25, 26, 27, 28, 29, or 30 amino acids long. In a more specific embodiment, the fragment of mutant KRAS is 18 amino acids long.

The arenavirus particle, wherein the region flanking the mutation at the N-terminus of the antigenic fragment is 0, 1, 2, 3, 4, 5, 6, 7, 8, 9, 10, 11, 12, 13, 14, 15, 16, 17, 18, 19, or 20 amino acids long. In a more specific embodiment, the region flanking the mutation at the N-terminus of the antigenic fragment is 8 or 9 amino acids long.

The arenavirus particle, wherein the region flanking the mutation at the C-terminus of the antigenic fragment is 0, 1, 2, 3, 4, 5, 6, 7, 8, 9, 10, 11, 12, 13, 14, 15, 16, 17, 18, 19, or 20 amino acids long. In a more specific embodiment, the region flanking the mutation at the C-terminus of the antigenic fragment is 8 or 9 amino acids long.

The arenavirus particle, wherein the nucleotide sequence encodes two, three, four, five, six, seven, eight, nine, 10, 11, 12, 13, 14, 15, 16, 17, 18, 19, or 20 antigenic fragments of a mutant KRAS, and wherein each antigenic fragment comprises the mutation of one of the mutant KRAS proteins. In a more specific embodiment, the nucleotide sequence encodes five antigenic fragments of a mutant KRAS, and wherein each antigenic fragment comprises the mutation of one of the mutant KRAS proteins. In a more specific embodiment, the five antigenic fragments of a mutant KRAS comprise the mutations G13D, G12V, G12C, G12D, and G12R.

The arenavirus particle, wherein the antigenic fragments comprise the same or different mutations of mutant KRAS proteins.

The arenavirus particle, wherein the antigenic fragments are fused to each other via the same or different linkers.

The arenavirus particle, wherein the antigenic fragments are fused directly to each other without intervening sequences.

The arenavirus particle, wherein the linker is AAY linker (AAY), AAA linker (AAA), GS linker (GGSGGGGSGG) (SEQ ID NO:42), or variants of AAY, AAA, and GS linker sequences optimized via in silico prediction.

The arenavirus particle, wherein the nucleotide sequence is engineered to reduce or remove any CpG and TpA islands.

The arenavirus particle, wherein the removal of the CpG and TpA islands comprises three cycles:
(i) CpG is removed in a first cycle;
(ii) TpA is removed in a second cycle; and
(iii) CpG is removed in a third cycle to remove newly introduced CpG in the second cycle.

The arenavirus particle, wherein the arenavirus genome comprises:
(i) a first S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral glycoprotein (“GP”) under control of an arenavirus genomic 3′ UTR; and
(iii) an L-Segment.

The arenavirus particle, wherein the arenavirus genome comprises:
(i) a first S-Segment comprising the nucleotide sequence of SEQ ID NO:20 under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence of SEQ ID NO:20 under control of an arenavirus genomic 5′ UTR and an ORF encoding the arenaviral glycoprotein (“GP”) under control of an arenavirus genomic 3′ UTR; and
(iii) an L-Segment.

The arenavirus particle, wherein the arenavirus genome comprises:
(i) a first S-Segment comprising the nucleotide sequence of SEQ ID NO:21; and
(ii) a second S-Segment comprising the nucleotide sequence of SEQ ID NO:22; and
(iii) an L-Segment.

The arenavirus particle, wherein the arenavirus genome comprises:
(i) a first S-Segment comprising the nucleotide sequence of SEQ ID NO:23; and
(ii) a second S-Segment comprising the nucleotide sequence of SEQ ID NO:24; and
(iii) an L-Segment.

The arenavirus particle, wherein the arenavirus genome comprises:
(i) a first S-Segment comprising an ORF encoding the arenavirus GP1 and GP2 subunits fused to a heterologous signal peptide under control of an arenavirus genomic 5′ UTR and an ORF encoding a fusion of arenavirus GP signal peptide and a nucleotide sequence encoding the antigenic fragment(s) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(iii) an L-Segment.

The arenavirus particle, wherein the nucleotide sequence encoding the antigenic fragment(s) on the first S-Segment is different from the nucleotide sequence encoding the antigenic fragment(s) on the second S-Segment.

The arenavirus particle, wherein the nucleotide sequence encoding the antigenic fragment(s) on the first S-Segment is the same as the nucleotide sequence encoding the antigenic fragment(s) on the second S-Segment.

The arenavirus particle, wherein the antigenic fragment(s) encoded on the first S-Segment is/are different from the antigenic fragment(s) encoded on the second S-Segment.

The arenavirus particle, wherein the antigenic fragment(s) encoded on the first S-Segment is/are the same as the antigenic fragment(s) encoded on the second S-Segment.

The arenavirus particle, wherein the antigenic fragments encoded on the first S-Segment are the same as the antigenic fragments encoded on the second S-Segment but are fused to each other in a different order from the order in which the antigenic fragments encoded on the second S-Segment are fused to each other.

The arenavirus particle, wherein the arenavirus genome further comprises a nucleotide sequence encoding an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation.

The arenavirus particle, wherein the arenavirus genome further comprises a nucleotide sequence encoding an antigenic fragment of mutant PI3KCA, wherein the antigenic fragment comprises the E545K, H1047R and/or E542K mutation.

The arenavirus particle, wherein the arenavirus genome further comprises a nucleotide sequence encoding an antigenic fragment of mutant BRAF, wherein the antigenic fragment comprises the V600E mutation.

The arenavirus particle, wherein the arenavirus genome further comprises a nucleotide sequence encoding an antigenic fragment of mutant U2AF1, wherein the antigenic fragment comprises the S34F mutation.

The arenavirus particle, wherein the arenavirus genome further comprises a nucleotide sequence encoding an antigenic fragment of mutant TP53, wherein the antigenic fragment comprises the G245S, Y220C, R248Q, R282W, H179R, V157F, R273C, R213L, R273H, R273L, R175H, R158L, R196P, R248W and/or C277F.

The arenavirus particle, wherein the arenavirus genome further comprises a nucleotide sequence encoding an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family.

The arenavirus particle, wherein the arenavirus particle is derived from lymphocytic choriomeningitis virus (LCMV) or Pichinde virus.

A pharmaceutical composition comprising the arenavirus particle.

A set of one or more nucleic acids encoding the genome of the arenavirus particle.

A host cell comprising the set of one or more nucleic acids encoding the genome of the arenavirus particle.

A method of making the arenavirus particle, wherein the method comprises culturing the host cell, and harvesting the arenavirus particle.

A method for treating a neoplastic disease in a subject in need thereof, wherein the method comprises administering to the subject an arenavirus particle, wherein
a. the arenavirus particle comprises an arenavirus genome comprising a nucleotide sequence encoding an antigenic fragment of mutant KRAS, wherein the antigenic fragment comprises the mutation; and
b. at least one arenavirus open reading frame (“ORF”) of the arenavirus genome is either (i) functionally inactivated or deleted; or (ii) located in a position other than the wild-type position of said at least one arenavirus ORF; or (iii) sequestered into two or more functional fragments and a fragment of the at least one arenavirus ORF is located in a position other than the wild-type position of said at least one arenavirus ORF.

The method for treating a neoplastic disease in a subject in need thereof, wherein the mutation in KRAS is at amino acid position G12, G13, A18, A59, Q61, K117, A146, or D119 of KRAS.

The method for treating a neoplastic disease in a subject in need thereof, wherein the mutation in KRAS is A18D, A59E, A59G, A59P, A59T, A59S, A59V, A146P, A146S, A146T, A146V, D119N, G12A, G12C, G12D, G12F, G12L, G12R, G12S, G12V, G13A, G13C, G13D, G13E, G13R, G13S, G13V, K117N, Q61E, Q61H, Q61K, Q61L, Q61P, Q61R or a combination thereof.

The method for treating a neoplastic disease in a subject in need thereof, wherein the mutation in KRAS is G12A, G12C, G12D, G12R, G12S, G12V, G13D, Q61H, Q61R, A146T or a combination thereof.

The method for treating a neoplastic disease in a subject in need thereof, wherein the mutation in KRAS is G13D, G12V, G12C, G12D, G12R or a combination thereof. In a more specific embodiment, the arenavirus genome comprises a nucleotide sequence encoding from N—to C-terminus fragments of mutant KRAS comprising the mutations G13D, G12V, G12C, G12D, and G12R, respectively. In other more specific embodiments, the arenavirus genome comprises a nucleotide sequence encoding fragments of mutant KRAS comprising the mutations G13D, G12V, G12C, G12D, and G12R in any possible order.

The method for treating a neoplastic disease in a subject in need thereof, wherein the arenavirus genome comprises a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of SEQ ID NO:20.

The method for treating a neoplastic disease in a subject in need thereof, wherein the arenavirus genome comprises a nucleotide sequence encoding an expression product whose amino acid sequence is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:19.

The method for treating a neoplastic disease in a subject in need thereof, wherein the antigenic fragment of mutant KRAS is 8, 9, 10, 11, 12, 13, 14, 15, 16, 17, 18, 19, 20, 21, 22, 23, 24, 25, 26, 27, 28, 29, or 30 amino acids long. In a more specific embodiment, the antigenic fragment of mutant KRAS is 18 amino acids long.

The method for treating a neoplastic disease in a subject in need thereof, wherein the region flanking the mutation at the N-terminus of the antigenic fragment is 0,1, 2, 3, 4, 5, 6, 7, 8, 9, 10, 11, 12, 13, 14, 15, 16, 17, 18, 19, or 20 amino acids long. In a more specific embodiment, the region flanking the mutation at the N-terminus of the antigenic fragment is 8 or 9 amino acids long.

The method for treating a neoplastic disease in a subject in need thereof, wherein the region flanking the mutation at the C-terminus of the antigenic fragment is 0,1, 2, 3, 4, 5, 6, 7, 8, 9, 10, 11, 12, 13, 14, 15, 16, 17, 18, 19, or 20 amino acids long. In a more specific embodiment, the region flanking the mutation at the C-terminus of the antigenic fragment is 8 or 9 amino acids long.

The method for treating a neoplastic disease in a subject in need thereof, wherein the nucleotide sequence encodes two, three, four, five, six, seven, eight, nine, 10, 11, 12, 13, 14, 15, 16, 17, 18, 19, or 20 antigenic fragments of mutant KRAS, wherein each antigenic fragment comprises the mutation of one of the mutant KRAS proteins. The method for treating a neoplastic disease in a subject in need thereof, wherein the nucleotide sequence encodes five antigenic fragments of mutant KRAS, wherein each antigenic fragment comprises the mutation of one of the mutant KRAS proteins. The method for treating a neoplastic disease in a subject in need thereof, wherein the five antigenic fragments of a mutant KRAS comprise the mutations G13D, G12V, G12C, G12D, and G12R.

The method for treating a neoplastic disease in a subject in need thereof, wherein the antigenic fragments comprise the same or different mutations of mutant KRAS proteins.

The method for treating a neoplastic disease in a subject in need thereof, wherein the antigenic fragments are fused to each other via the same or different linkers.

The method for treating a neoplastic disease in a subject in need thereof, wherein the antigenic fragments are fused directly to each other without intervening sequences.

The method for treating a neoplastic disease in a subject in need thereof, wherein the linker is AAY linker (AAY), AAA linker (AAA), GS linker (GGSGGGGSGG) (SEQ ID NO:42), or variants of AAY, AAA, and GS linker sequences optimized via in silico prediction.

The method for treating a neoplastic disease in a subject in need thereof, wherein the nucleotide sequence is engineered to reduce or remove any CpG and TpA islands.

The method for treating a neoplastic disease in a subject in need thereof, wherein the removal of the CpG and TpA islands comprises three cycles:
(i) CpG is removed in a first cycle;
(ii) TpA is removed in a second cycle; and
(iii) CpG is removed in a third cycle to remove newly introduced CpG in the second cycle.

The method for treating a neoplastic disease in a subject in need thereof, wherein the arenavirus genome comprises:
(i) a first S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral glycoprotein (“GP”) under control of an arenavirus genomic 3′ UTR; and
(iii) an L-Segment.

The method for treating a neoplastic disease in a subject in need thereof, wherein the arenavirus genome comprises:
(i) a first S-Segment comprising the nucleotide sequence of SEQ ID NO:20 under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence of SEQ ID NO:20 under control of an arenavirus genomic 5′ UTR and an ORF encoding the arenaviral glycoprotein (“GP”) under control of an arenavirus genomic 3′ UTR; and
(iii) an L-Segment.

The method for treating a neoplastic disease in a subject in need thereof, wherein the arenavirus genome comprises:
(i) a first S-Segment comprising the nucleotide sequence of SEQ ID NO:21; and
(ii) a second S-Segment comprising the nucleotide sequence of SEQ ID NO:22; and
(iii) an L-Segment.

The method for treating a neoplastic disease in a subject in need thereof, wherein the arenavirus genome comprises:
(i) a first S-Segment comprising the nucleotide sequence of SEQ ID NO:23; and
(ii) a second S-Segment comprising the nucleotide sequence of SEQ ID NO:24; and
(iii) an L-Segment.

The method for treating a neoplastic disease in a subject in need thereof, wherein the arenavirus genome comprises:
(i) a first S-Segment comprising an ORF encoding the arenavirus GP1 and GP2 subunits fused to a heterologous signal peptide under control of an arenavirus genomic 5′ UTR and an ORF encoding a fusion of arenavirus GP signal peptide and a nucleotide sequence encoding the antigenic fragment(s) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(iii) an L-Segment.

The method for treating a neoplastic disease in a subject in need thereof, wherein the nucleotide sequence encoding the antigenic fragment(s) on the first S-Segment is different from the nucleotide sequence encoding the antigenic fragment(s) on the second S-Segment.

The method for treating a neoplastic disease in a subject in need thereof, wherein the nucleotide sequence encoding the antigenic fragment(s) on the first S-Segment is the same as the nucleotide sequence encoding the antigenic fragment(s) on the second S-Segment.

The method for treating a neoplastic disease in a subject in need thereof, wherein the antigenic fragment(s) encoded on the first S-Segment is/are different from the antigenic fragment(s) encoded on the second S-Segment.

The method for treating a neoplastic disease in a subject in need thereof, wherein the antigenic fragment(s) encoded on the first S-Segment is/are the same as the antigenic fragment(s) encoded on the second S-Segment.

The method for treating a neoplastic disease in a subject in need thereof, wherein the antigenic fragments encoded on the first S-Segment are the same as the antigenic fragments encoded on the second S-Segment but are fused to each other in a different order from the order in which the antigenic fragments encoded on the second S-Segment are fused to each other.

The method for treating a neoplastic disease in a subject in need thereof, wherein the neoplastic disease is pancreatic cancer, colorectal cancer, lung adenocarcinoma, lung squamous cell carcinoma, or non-small cell lung cancer (NSCLC).

The method for treating a neoplastic disease in a subject in need thereof, wherein the arenavirus genome comprises a nucleotide sequence encoding antigenic fragments of mutant KRAS, wherein the antigenic fragments comprise the mutation, wherein the mutation in KRAS is G12D, G12V, G12R, Q61H, Q61R, G12C, G12S, and/or G12A and wherein the neoplastic disease is pancreatic cancer.

The method for treating a neoplastic disease in a subject in need thereof, wherein the arenavirus genome further comprises a nucleotide sequence encoding an antigenic fragment of mutant TP53, and/or mutant U2AF1, wherein the antigenic fragment comprises the mutation, wherein the mutation in KRAS is G12D, G12V, G12R, Q61H and/or Q61R, wherein the mutation in TP53 is R175H, R248W, G245S, R282W, R248Q and/or R273C, wherein the mutation in U2AF1 is S34F, and wherein the neoplastic disease is pancreatic cancer.

The method for treating a neoplastic disease in a subject in need thereof, wherein the arenavirus genome comprises a nucleotide sequence encoding antigenic fragments of mutant KRAS, wherein the antigenic fragment comprises the mutation, and wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma.

The method for treating a neoplastic disease in a subject in need thereof, wherein the arenavirus genome comprises a nucleotide sequence encoding antigenic fragments of mutant KRAS, wherein the antigenic fragment comprises the mutation, and wherein the mutation in KRAS is G13D, G12V, G12C, G12D, and G12R and wherein the neoplastic disease is pancreatic cancer, colorectal cancer or lung adenocarcinoma.

The method for treating a neoplastic disease in a subject in need thereof, wherein the arenavirus genome further comprises a nucleotide sequence encoding an antigenic fragment of mutant TP53, wherein the antigenic fragment comprises the mutation, wherein the mutation in KRAS is G12D, G12R, G12V, Q61H and/or Q61R, wherein the mutation in TP53 is R175H, R248W, G245S, R282W, R248Q and/or R273C, and wherein the neoplastic disease is pancreatic cancer.

The method for treating a neoplastic disease in a subject in need thereof, wherein the arenavirus genome further comprises a nucleotide sequence encoding an antigenic fragment of mutant BRAF, mutant TP53, mutant FBXW7, and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is colorectal cancer or lung adenocarcinoma.

The method for treating a neoplastic disease in a subject in need thereof, wherein the arenavirus genome further comprises a nucleotide sequence encoding an antigenic fragment of mutant BRAF, mutant PIK3CA, mutant EGFR, mutant TP53, and/or mutant U2AF1, wherein the antigenic fragment comprises the mutation, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, wherein the mutation in U2AF1 is S34F, and wherein the neoplastic disease is pancreatic cancer, colorectal cancer or lung adenocarcinoma.

The method for treating a neoplastic disease in a subject in need thereof, wherein the arenavirus genome further comprises a nucleotide sequence encoding an antigenic fragment of mutant TP53, mutant U2AF1, mutant PIK3CA, mutant EGFR and/or mutant BRAF, wherein the antigenic fragment comprises the mutation, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E, and wherein the neoplastic disease is lung adenocarcinoma.

The method for treating a neoplastic disease in a subject in need thereof, wherein the arenavirus genome further comprises a nucleotide sequence encoding an antigenic fragment of mutant AKT1, mutant BRAF, mutant HER2, mutant MEK1, mutant MET, mutant NRAS, mutant PIK3CA and/or mutant RET, wherein the antigenic fragment comprises the mutation, and wherein the neoplastic disease is non-small cell lung cancer (NSCLC).

The method for treating a neoplastic disease in a subject in need thereof, wherein the arenavirus genome further comprises a nucleotide sequence encoding an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation.

The method for treating a neoplastic disease in a subject in need thereof, wherein the arenavirus genome further comprises a nucleotide sequence encoding an antigenic fragment of mutant PI3KCA, wherein the antigenic fragment comprises the mutation, and wherein the mutation in PI3KCA is E545K, H1047R and/or E542K.

The method for treating a neoplastic disease in a subject in need thereof, wherein the arenavirus genome further comprises a nucleotide sequence encoding an antigenic fragment of mutant BRAF, wherein the antigenic fragment comprises the mutation, and wherein the mutation in BRAF is V600E.

The method for treating a neoplastic disease in a subject in need thereof, wherein the arenavirus genome further comprises a nucleotide sequence encoding an antigenic fragment of mutant U2AF1, wherein the antigenic fragment comprises the mutation, and wherein the mutation in U2AF1 is S34F.

The method for treating a neoplastic disease in a subject in need thereof, wherein the arenavirus genome further comprises a nucleotide sequence encoding an antigenic fragment of mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W and/or C277F.

The method for treating a neoplastic disease in a subject in need thereof, wherein the arenavirus genome further comprises a nucleotide sequence encoding an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family.

The method for treating a neoplastic disease in a subject in need thereof, wherein the method further comprises administering a second arenavirus particle, wherein the second arenavirus particle encodes an antigenic fragment of mutant KRAS, wherein the antigenic fragments comprise the mutation, wherein the mutation in KRAS is G12D, G12V, G12R, Q61H, Q61R, G12C, G12S, and/or G12A and wherein the neoplastic disease is pancreatic cancer.

The method for treating a neoplastic disease in a subject in need thereof, wherein the method further comprises administering a second arenavirus particle, wherein the second arenavirus particle encodes an antigenic fragment of mutant KRAS, mutant TP53, and/or mutant U2AF1, wherein the antigenic fragment comprises the mutation, wherein the mutation in KRAS is G12D, G12V, G12R, Q61H and/or Q61R, wherein the mutation in TP53 is R175H, R248W, G245S, R282W, R248Q and/or R273C, wherein the mutation in U2AF1 is S34F, and wherein the neoplastic disease is pancreatic cancer.

The method for treating a neoplastic disease in a subject in need thereof, wherein the method further comprises administering a second arenavirus particle, wherein the second arenavirus particle encodes an antigenic fragment of mutant KRAS, wherein the antigenic fragment comprises the mutation, and wherein the neoplastic disease is pancreatic cancer.

The method for treating a neoplastic disease in a subject in need thereof, wherein the method further comprises administering a second arenavirus particle, wherein the second arenavirus particle encodes an antigenic fragment of mutant KRAS, wherein the antigenic fragment comprises the mutation, and wherein the neoplastic disease is colorectal cancer.

The method for treating a neoplastic disease in a subject in need thereof, wherein the method further comprises administering a second arenavirus particle, wherein the second arenavirus particle encodes an antigenic fragment of mutant KRAS, wherein the antigenic fragment comprises the mutation, and wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma.

The method for treating a neoplastic disease in a subject in need thereof, wherein the method further comprises administering a second arenavirus particle, wherein the second arenavirus particle encodes an antigenic fragment of mutant KRAS and/or mutant TP53, wherein the antigenic fragment comprises the mutation, wherein the mutation in KRAS is G12D, G12R, G12V Q61H and/or Q61R, wherein the mutation in TP53 is R175H, R248W, G245S, R282W, R248Q and/or R273C, and wherein the neoplastic disease is pancreatic cancer.

The method for treating a neoplastic disease in a subject in need thereof, wherein the method further comprises administering a second arenavirus particle, wherein the second arenavirus particle encodes an antigenic fragment of mutant KRAS, wherein the antigenic fragment comprises the mutation, and wherein the mutation in KRAS is G13D, G12V, G12C, G12D, and G12R and wherein the neoplastic disease is pancreatic cancer, colorectal cancer or lung adenocarcinoma.

The method for treating a neoplastic disease in a subject in need thereof, wherein the method further comprises administering a second arenavirus particle, wherein the second arenavirus particle encodes an antigenic fragment of mutant KRAS, mutant BRAF, mutant TP53, mutant FBXW7, and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is colorectal cancer or lung adenocarcinoma.

The method for treating a neoplastic disease in a subject in need thereof, wherein the method further comprises administering a second arenavirus particle, wherein the second arenavirus particle encodes an antigenic fragment of mutant KRAS, mutant BRAF, mutant PIK3CA, mutant EGFR, mutant TP53, and/or mutant U2AF1, wherein the antigenic fragment comprises the mutation, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, wherein the mutation in U2AF1 is S34F, and wherein the neoplastic disease is pancreatic cancer, colorectal cancer or lung adenocarcinoma.

The method for treating a neoplastic disease in a subject in need thereof, wherein the method further comprises administering a second arenavirus particle, wherein the second arenavirus particle encodes an antigenic fragment of mutant KRAS, mutant TP53, mutant U2AF1, mutant PIK3CA, mutant EGFR and/or mutant BRAF, wherein the antigenic fragment comprises the mutation, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E, and wherein the neoplastic disease is lung adenocarcinoma.

The method for treating a neoplastic disease in a subject in need thereof, wherein the method further comprises administering a second arenavirus particle, wherein the second arenavirus particle encodes an antigenic fragment of mutant TP53, and wherein the antigenic fragment comprises the mutation.

The method for treating a neoplastic disease in a subject in need thereof, wherein the method further comprises administering a second arenavirus particle, wherein the second arenavirus particle encodes an antigenic fragment of mutant KRAS, mutant AKT1, mutant BRAF, mutant HER2, mutant MEK1, mutant MET, mutant NRAS, mutant PIK3CA and/or mutant RET, wherein the antigenic fragment comprises the mutation, and wherein the neoplastic disease is non-small cell lung cancer (NSCLC).

The method for treating a neoplastic disease in a subject in need thereof, wherein the method further comprises administering a second arenavirus particle, wherein the second arenavirus particle encodes an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation.

The method for treating a neoplastic disease in a subject in need thereof, wherein the method further comprises administering a second arenavirus particle, wherein the second arenavirus particle encodes an antigenic fragment of mutant PI3KCA, wherein the antigenic fragment comprises the mutation, and wherein the mutation in PI3KCA is E545K, H1047R and/or E542K.

The method for treating a neoplastic disease in a subject in need thereof, wherein the method further comprises administering a second arenavirus particle, wherein the second arenavirus particle encodes an antigenic fragment of mutant BRAF, wherein the antigenic fragment comprises the mutation, and wherein the mutation in BRAF is V600E.

The method for treating a neoplastic disease in a subject in need thereof, wherein the method further comprises administering a second arenavirus particle, wherein the second arenavirus particle encodes an antigenic fragment of mutant U2AF1, wherein the antigenic fragment comprises the mutation, and wherein the mutation in U2AF1 is S34F.

The method for treating a neoplastic disease in a subject in need thereof, wherein the method further comprises administering a second arenavirus particle, wherein the second arenavirus particle encodes an antigenic fragment of mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W and/or C277F.

The method for treating a neoplastic disease in a subject in need thereof, wherein the method further comprises administering a second arenavirus particle, wherein the second arenavirus particle encodes an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family.

The method for treating a neoplastic disease in a subject in need thereof, wherein the method further comprises administering a second arenavirus particle, wherein the second arenavirus particle comprises an arenavirus genome comprising the nucleotide sequences of SEQ ID NOs:21 and 22. The method for treating a neoplastic disease in a subject in need thereof, wherein the method further comprises administering a second arenavirus particle, wherein the second arenavirus particle comprises an arenavirus genome comprising the nucleotide sequences of SEQ ID NOs:23 and 24.

The method for treating a neoplastic disease in a subject in need thereof, wherein the arenavirus particle is derived from lymphocytic choriomeningitis virus (LCMV) or Pichinde virus.

The method for treating a neoplastic disease in a subject in need thereof, wherein the neoplastic disease is a solid tumor, and wherein the method results in an increase of the concentration of T cells within the solid tumor.

The method for treating a neoplastic disease in a subject in need thereof, wherein the neoplastic disease is acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Szary syndrome); testicular cancer; throat cancer; thymoma; thymoma and thymic carcinoma; thyroid cancer; childhood thyroid cancer; transitional cell cancer of the renal pelvis and ureter; urethral cancer; uterine cancer, endometrial; uterine sarcoma; vaginal cancer; vulvar cancer; and Wilms tumor.

The method for treating a neoplastic disease in a subject in need thereof, wherein the neoplastic disease is a solid tumor, and wherein the route of administration of the arenavirus particle is via intratumoral injection.

### 3.1 Conventions and Abbreviations

| | |
|---|---|
| APC | Antigen presenting cells |
| C-cell | Complementing cell line |
| CD4 | Cluster of Differentiation 4 |
| CD8 | Cluster of Differentiation 8 |
| CMI | Cell-mediated immunity |
| GP | Glycoprotein |
| HRP | Horse radish peroxidase |
| IFN-γ | Interferon-γ |
| LCMV | Lymphocytic choriomeningitis virus |
| MHC | Major Histocompatibility Complex |
| NP | Nucleoprotein |
| ORF | Open reading frame |
| PICV | Pichinde virus |
| TNF-α | Tumor necrosis factor-α |
| UTR | Untranslated region |
| z | Matrix Protein Z |

### 4. BRIEF DESCRIPTION OF THE FIGURES

FIG. 1: Schematic representation of artLCMV-4xKRASmut and artPICV-4xKRASmut vectors.

FIG. 2: Schematic representation of artLCMV-4xKRASmut_E7 and artPICV-4xKRASmut_E7 vectors.

FIG. 3: Schematic representation of artLCMV-4xKRASmut_EBV and artPICV-4xKRASmut_EBV vectors.

FIG. 4: Schematic representation of artLCMV-KRASmut and artPICV-KRASmut vectors.

FIGS. 5A and 5B: Schematic representation of artLCMV-14xp53mut (FIG. 5A) and artPICV-14xp53mut (FIG. 5B) vectors.

FIGS. 6A and 6B: Schematic representation of artLCMV-14xp53mut_E7 (FIG. 6A) and artPICV-14xp53mut_E7 (FIG. 6B) vectors.

FIGS. 7A and 7B: Schematic representation of artLCMV-14xp53mut_EBV (FIG. 7A) and artPICV-14xp53mut_EBV (FIG. 7B) vectors.

FIG. 8: Schematic representation of artLCMV-p53mut and artPICV-p53mut vectors.

FIG. 9A: Schematic representation of artLCMV-p53mut/KRASmut and artPICV-p53mut/KRASmut vectors.

FIG. 9B: Schematic representation of artLCMV-KRASmut/p53mut and artPICV-KRASmut/p53mut vectors.

FIG. 9C: Schematic representation of antigenic inserts in artLCMV and artPICV vectors encoding antigenic fragments of mutant KRAS and/or TP53.

FIG. 10: Schematic representation of artLCMV-5xKRASmut-H2 and artPICV-5xKRASmut-H2 vectors.

FIG. 11: Induction of CD8+ T cell response in mice transgenic for HLA-A*11 (i.e., CB6F1-Tg(HLA-A*1101/H2-Kb)A11.01 mice) after administration of arenavirus particles encoding different combinations of mutated KRAS epitopes. CD8+ T cell (i.e., IFN-γ+) responses against individual mutated KRAS epitopes were analyzed by ELISPOT in HLA-A*11 transgenic mice after prime-boost administration of the indicated vectors. Peptide stimulation was performed with wild-type and mutation-specific KRAS-based peptides. A mixture of NP-based peptides derived from LCMV and PICV was used as control. IFN-γ+ SFU (per 10⁵ cells) are shown for individual mice, as arithmetic means±standard deviation.

FIGS. 12A and 12B: Transgene stability of artLCMV-5xKRASmut-H2 (FIG. 12A) and artPICV-5xKRASmut-H2 (FIG. 12B). 5xKRASmut transgene stability was analyzed by PCR at indicated passage levels (p1-p6).

FIG. 13: Nucleotide sequence (SEQ ID NO:21) of artLCMV-5xKRASmut-H2-NP-S-segment (S Segment #1 as per FIG. 10). The following elements are indicated from 5′ to 3′ of the disclosed sequence. The 5′ untranslated region (UTR) based on LCMV c113 S-segment is shown in lowercase letters; the KRASmut-H2 transgene is shown in bold letters; the intergenic region (IGR) based on LCMV c113 S-segment is framed; the nucleoprotein (NP) based on LCMV c113 is shown in underlined letters; the 3′ untranslated region (UTR) based on LCMV c113 S-segment is shown in lowercase letters. The genomic segment is RNA, the sequence in SEQ ID NO:21 is shown as DNA; however, exchanging all thymidines (“T”) in SEQ ID NO:21 for uridines (“U”) provides the RNA sequence (SEQ ID NO:34).

FIG. 14: Nucleotide sequence (SEQ ID NO:22) of artLCMV-5xKRASmut-H2-GP-S-segment (S Segment #2 as per FIG. 10). The following elements are indicated from 5′ to 3′ of the disclosed sequence. The 5′ untranslated region (UTR) based on LCMV c113 S-segment is shown in lowercase letters; the KRASmut-H2 transgene is shown in bold; the intergenic region (IGR) based on LCMV c113 S-segment is shown in box; the glycoprotein (GP) based on LCMV WE is shown in underline; the 3′ untranslated region (UTR) based on LCMV c113 S-segment is shown in lowercase letters. The genomic segment is RNA, the sequence in SEQ ID NO:22 is shown for DNA; however, exchanging all thymidines (“T”) in SEQ ID NO:22 for uridines (“U”) provides the RNA sequence (SEQ ID NO:35).

FIG. 15: Nucleotide sequence (SEQ ID NO:23) of artPICV-5xKRASmut-H2-NP-S-segment (S Segment #1 as per FIG. 10). The following elements are indicated from 5′ to 3′ of the disclosed sequence. The 5′ untranslated region (UTR) based on PICV p18 S-segment is shown in lowercase letters; the KRASmut-H2 transgene is shown in bold; the intergenic region (IGR) based on PICV p18 S-segment is shown in box; the nucleoprotein (NP) based on PICV p18 is shown in underline; the 3′ untranslated region (UTR) based on PICV p18 S-segment is shown in lowercase letters. The genomic segment is RNA, the sequence in SEQ ID NO:23 is shown for DNA; however, exchanging all thymidines (“T”) in SEQ ID NO:23 for uridines (“U”) provides the RNA sequence (SEQ ID NO:36).

FIG. 16: Nucleotide sequence (SEQ ID NO:24) of artPCIV-5xKRASmut-H2-GP-S-segment (S Segment #2 as per FIG. 10). The following elements are indicated from 5′ to 3′ of the disclosed sequence. The 5′ untranslated region (UTR) based on PICV p18 S-segment is shown in lowercase letters; the KRASmut-H2 transgene is shown in bold; the intergenic region (IGR) based on PICV p18 S-segment is shown in box; the glycoprotein (GP) based on PICV p18 is shown in underline; the 3′ untranslated region (UTR) based on PICV p18 S-segment is shown in lowercase letters. The genomic segment is RNA, the sequence in SEQ ID NO:24 is shown for DNA; however, exchanging all thymidines (“T”) in SEQ ID NO:24 for uridines (“U”) provides the RNA sequence (SEQ ID NO:37).

FIG. 17: Nucleotide sequence (SEQ ID NO:27) of artLCMV-5xKRASmut-H1-NP-S-segment (S Segment #1 as per FIG. 10). The following elements are indicated from 5′ to 3′ of the disclosed sequence. The 5′ untranslated region (UTR) based on LCMV c113 S-segment is shown in lowercase letters; the KRASmut-H1 transgene is shown in bold; the intergenic region (IGR) based on LCMV c113 S-segment is shown in box; the nucleoprotein (NP) based on LCMV c113 is shown in underline; the 3′ untranslated region (UTR) based on LCMV c113 S-segment is shown in lowercase letters. The genomic segment is RNA, the sequence in SEQ ID NO:27 is shown for DNA; however, exchanging all thymidines (“T”) in SEQ ID NO:27 for uridines (“U”) provides the RNA sequence (SEQ ID NO:38).

FIG. 18: Nucleotide sequence (SEQ ID NO:28) of artLCMV-5xKRASmut-H1-GP-S-segment (S Segment #2 as per FIG. 10). The following elements are indicated from 5′ to 3′ of the disclosed sequence. The 5′ untranslated region (UTR) based on LCMV c113 S-segment is shown in lowercase letters; the KRASmut-H1 transgene is shown in bold; the intergenic region (IGR) based on LCMV c113 S-segment is shown in box; the glycoprotein (GP) based on LCMV WE is shown in underline; the 3′ untranslated region (UTR) based on LCMV c113 S-segment is shown in lowercase letters. The genomic segment is RNA, the sequence in SEQ ID NO:28 is shown for DNA; however, exchanging all thymidines (“T”) in SEQ ID NO:28 for uridines (“U”) provides the RNA sequence (SEQ ID NO:39).

FIG. 19: Nucleotide sequence (SEQ ID NO:29) of artPICV-5xKRASmut-H1-NP-S-segment (S Segment #1 as per FIG. 10). The following elements are indicated from 5′ to 3′ of the disclosed sequence. The 5′ untranslated region (UTR) based on PICV p18 S-segment is shown in lowercase letters; the KRASmut-H1 transgene is shown in bold; the intergenic region (IGR) based on PICV p18 S-segment is shown in box; the nucleoprotein (NP) based on PICV p18 is shown in underline; the 3′ untranslated region (UTR) based on PICV p18 S-segment is shown in lowercase letters. The genomic segment is RNA, the sequence in SEQ ID NO:29 is shown for DNA; however, exchanging all thymidines (“T”) in SEQ ID NO:29 for uridines (“U”) provides the RNA sequence (SEQ ID NO:40).

FIG. 20: Nucleotide sequence (SEQ ID NO:30) of artPCIV-5xKRASmut-H1-GP-S-segment (S Segment #2 as per FIG. 10). The following elements are indicated from 5′ to 3′ of the disclosed sequence. The 5′ untranslated region (UTR) based on PICV p18 S-segment is shown in lowercase letters; the KRASmut-H1 transgene is shown in bold; the intergenic region (IGR) based on PICV p18 S-segment is shown in box; the glycoprotein (GP) based on PICV p18 is shown in underline; the 3′ untranslated region (UTR) based on PICV p18 S-segment is shown in lowercase letters. The genomic segment is RNA, the sequence in SEQ ID NO:30 is shown for DNA; however, exchanging all thymidines (“T”) in SEQ ID NO:30 for uridines (“U”) provides the RNA sequence (SEQ ID NO:41).

FIG. 21: Induction of CD8 T cell response in mice transgenic for HLA-B*07 (i.e., CB6F1-Tg(HLA-B*0702/H2-Kb)B7.xx mice) after administration of arenavirus particles encoding different combinations of mutated KRAS epitopes. CD8 T cell (i.e., IFN-γ+) responses against individual mutated KRAS epitopes were analyzed by ELISpot in HLA-B*07 transgenic mice after prime-boost administration of the indicated vectors. Peptide stimulation was performed with wild-type and mutation-specific KRAS-based peptides. A mixture of NP-based peptides derived from LCMV and PICV was used as control. IFN-γ+ SFU (per 10⁵ cells) are shown for individual mice, as arithmetic means±standard deviation.

FIG. 22: Study design of the working example disclosed in Section 8.5 of the present disclosure.

FIG. 23: Study design of the working example disclosed in Section 8.6 of the present disclosure.

FIG. 24: Induction of CD8 T cell response in mice transgenic for HLA-A*11 (i.e., CB6F1-Tg(HLA-A*1101/H2-Kb)A11.01 mice) after administration of arenavirus particles encoding combination of mutated KRAS epitopes. CD8 T cell (i.e., IFN-γ+) responses against individual mutated KRAS epitopes were analyzed by ELISpot in HLA-A*11 transgenic mice after prime administration of the indicated vectors. Peptide stimulation was performed with wild-type and mutation-specific KRAS-based peptides. A mixture of NP-based peptides derived from LCMV was used as control. NP-based peptides derived from PICV was not detected due to the a technical error. IFN-γ+ SFU (per 10⁵ cells) are shown for individual mice, as arithmetic means standard deviation.

### 5. DETAILED DESCRIPTION OF THE INVENTION

Provided herein are methods for treating a neoplastic disease (see section 5.1) in a subject in need thereof, wherein the method comprises delivering to the subject an arenavirus particle (see arenavirus particles specified in section 5.3-5.6), wherein the arenavirus particle is engineered to contain an arenavirus genome comprising a nucleotide sequence encoding an antigenic fragment of mutant KRAS (see section 5.7) alone or further comprising a nucleotide sequence encoding an antigenic fragment of a mutated cancer driver gene (see section 5.8, e.g., a mutant TP53), or a tumor-associated antigen (see section 5.9). In certain embodiments, the method for treating a neoplastic disease comprises administering a first arenavirus particle and a second arenavirus particle, wherein the first arenavirus particle and the second arenavirus particle encode the same or different antigenic fragments (see section 5.10).

In certain embodiments, the various antigenic fragments (from mutant KRAS, mutated cancer driver gene (e.g., a mutant TP53), and/or tumor-associated antigen) are present on the same transcript, are present in the same position of the arenavirus genome, are on the same genomic segment, and/or are present in the same arenavirus genome. In certain embodiments, the various antigenic fragments (from mutant KRAS, mutated cancer driver gene (e.g., a mutant TP53), and/or tumor-associated antigen) are present on separate transcripts, are present in separate positions of the arenavirus genome, are present on separate genomic segments, and/or are present in separate arenavirus genomes.

### 5.1 Neoplastic Diseases

In certain embodiments, the neoplastic diseases that can be treated with the methods and compositions described herein include the neoplastic diseases listed below. As such the mutant KRAS (see section 5.7), mutated cancer driver gene (see section 5.8, e.g., a mutant TP53), or tumor-associated antigen (see section 5.9) that is encoded by the genome of an arenaviral particle described herein can be associated with or can be specific to one of the listed neoplastic diseases. Neoplastic diseases include acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; childhood thyroid cancer; transitional cell cancer of the renal pelvis and ureter; urethral cancer; uterine cancer, endometrial; uterine sarcoma; vaginal cancer; vulvar cancer; and Wilms tumor.

In certain embodiments, the method for treating a neoplastic disease in a subject in need thereof comprises administering an arenavirus particle to the subject, wherein the arenavirus particle encodes an antigenic fragment of a mutant KRAS (see section 5.7) and/or a mutant TP53 (see section 5.8). In certain embodiments, the arenavirus particle encodes an antigenic fragment of mutant KRAS wherein the mutation in KRAS can be associated with or can be specific to pancreatic cancer, colorectal cancer, lung adenocarcinoma, lung squamous cell carcinoma, or non-small cell lung cancer (NSCLC). In certain embodiments, the method for treating a neoplastic disease in a subject in need thereof comprises administering an arenavirus particle to the subject, wherein the arenavirus particle encodes an antigenic fragment of a mutant KRAS, wherein the mutation in KRAS can be associated with or can be specific to pancreatic cancer, colorectal cancer, lung adenocarcinoma, wherein the mutation in KRAS is G12D, G12V, G12C, G12R, G13D, A146T, G12S, Q61H, G12A, and/or Q61R. In certain embodiments, the method for treating a neoplastic disease in a subject in need thereof comprises administering an arenavirus particle to the subject, wherein the arenavirus particle encodes an antigenic fragment of a mutant KRAS, wherein the mutation in KRAS can be associated with or can be specific to pancreatic cancer, colorectal cancer, lung adenocarcinoma, wherein the mutation in KRAS is G13D, G12V, G12C, G12D, and G12R. In certain embodiments, the method for treating a neoplastic disease in a subject in need thereof comprises administering an arenavirus particle to the subject, wherein the arenavirus particle encodes an antigenic fragment of a mutant KRAS, wherein the mutation in KRAS can be associated with or can be specific to pancreatic cancer, wherein the mutation in KRAS is G12D, G12V, G12R, Q61H, Q61R, G12C, G12S, and/or G12A. In certain embodiments, the method for treating a neoplastic disease in a subject in need thereof comprises administering an arenavirus particle to the subject, wherein the arenavirus particle encodes an antigenic fragment of a mutant KRAS and/or mutant TP53, wherein the mutation in KRAS and TP53 can be associated with or can be specific to pancreatic cancer, wherein the mutation in KRAS is G12D, G12R and/or G12V, wherein the mutation in TP53 is R175H, R248W and/or R273C. In certain embodiments, the method for treating a neoplastic disease in a subject in need thereof comprises administering an arenavirus particle to the subject, wherein the arenavirus particle encodes an antigenic fragment of a mutant KRAS and/or mutant TP53, wherein the antigenic fragment comprises the mutation, wherein the mutation in KRAS and TP53 can be associated with or can be specific to colorectal cancer or lung adenocarcinoma, wherein the mutation in KRAS is G12D, G12V, G13D, A146T and/or G12C, wherein the mutation in in TP53 is R175H, R273H and/or R248W. In certain embodiments, the arenavirus particle encodes an antigenic fragment of mutant KRAS and/or mutant TP53, wherein the antigenic fragment comprises the mutation, wherein the mutation in KRAS and TP53 can be associated with or can be specific to lung adenocarcinoma, wherein the mutation in KRAS is G12C, G12V, G12D, and/or G12S, wherein the mutation in TP53 is R175H. In certain embodiments, the arenavirus particle encodes an antigenic fragment of mutant KRAS and/or mutant TP53, wherein the antigenic fragment comprises the mutation, wherein the mutation in KRAS and TP53 can be associated with or can be specific to pancreatic cancer, colorectal cancer or lung adenocarcinoma, wherein the mutation in KRAS is G12C, G12D, G12R, G13D and/or G12V, wherein the mutation in TP53 is R175H, R273H and/or R248W.

In certain embodiments, the method for treating a neoplastic disease in a subject in need thereof further comprises administering a second arenavirus particle to the subject, wherein the second arenavirus particle encodes an antigenic fragment of a mutant KRAS (see section 5.7) and/or a mutant TP53 (see section 5.8). In certain embodiments, the second arenavirus particle encodes an antigenic fragment of mutant KRAS wherein the mutation in KRAS can be associated with or can be specific to pancreatic cancer. In certain embodiments, the second arenavirus particle encodes an antigenic fragment of mutant KRAS wherein the mutation in KRAS can be associated with or can be specific to pancreatic cancer, wherein the mutation in KRAS is G12D, G12V, G12R, Q61H, Q61R, G12C, G12S, and/or G12A. In certain embodiments, the second arenavirus particle encodes an antigenic fragment of mutant KRAS wherein the mutation in KRAS can be associated with or can be specific to pancreatic cancer, colorectal cancer, lung adenocarcinoma, wherein the mutation in KRAS is G12D, G12V, G12C, G12R, G13D, A146T, G12S, Q61H, G12A, and/or Q61R. In certain embodiments, the second arenavirus particle encodes an antigenic fragment of mutant KRAS and/or mutant TP53, wherein the mutation in KRAS and TP53 can be associated with or can be specific to pancreatic cancer, wherein the mutation in KRAS is G12D, G12R and/or G12V, wherein the mutation in TP53 is R175H, R248W and/or R273C. In certain embodiments, the second arenavirus particle encodes an antigenic fragment of mutant KRAS and/or mutant TP53, wherein the mutation in KRAS and TP53 can be associated with or can be specific to colorectal cancer or lung adenocarcinoma, wherein the mutation in KRAS is G12D, G12V, G13D, A146T and/or G12C, wherein the mutation in TP53 is R175H, R273H and/or R248W. In certain embodiments, the second arenavirus particle encodes an antigenic fragment of mutant KRAS and/or mutant TP53, wherein the mutation in KRAS and TP53 can be associated with or can be specific to pancreatic cancer, colorectal cancer, or lung adenocarcinoma, wherein the mutation in KRAS is G12C, G12D, G12R, G13D and/or G12V, wherein the mutation in TP53 is R175H, R273H and/or R248W. In certain embodiments, the second arenavirus particle encodes an antigenic fragment of mutant KRAS and/or mutant TP53, wherein the mutation in KRAS and TP53 can be associated with or can be specific to lung adenocarcinoma, wherein the mutation in KRAS is G12C, G12V, G12D, and/or G12S, wherein the mutation in TP53 is R175H.

In certain embodiments, the method for treating a neoplastic disease in a subject in need thereof comprises administering an arenavirus particle to the subject, wherein the arenavirus particle encodes an antigenic fragment of a mutant KRAS (see section 5.7), a mutated cancer driver gene (see section 5.8), or a tumor-associated antigen (see section 5.9). In certain embodiments, the arenavirus particle encodes an antigenic fragment of mutant KRAS wherein the mutation in KRAS can be associated with or can be specific to pancreatic cancer, colorectal cancer, lung adenocarcinoma, lung squamous cell carcinoma, or non-small cell lung cancer (NSCLC). In certain embodiments, the method for treating a neoplastic disease in a subject in need thereof comprises administering an arenavirus particle to the subject, wherein the arenavirus particle encodes an antigenic fragment of a mutant KRAS, wherein the mutation in KRAS can be associated with or can be specific to pancreatic cancer, and wherein the mutation in KRAS is KRAS is G12D, G12V, G12R, Q61H, Q61R, G12C, G12S, and/or G12A. In certain embodiments, the method for treating a neoplastic disease in a subject in need thereof comprises administering an arenavirus particle to the subject, wherein the arenavirus particle encodes an antigenic fragment of a mutant KRAS, mutant TP53, and/or mutant U2AF1, wherein the mutation in KRAS, TP53 and/or U2AF1 can be associated with or can be specific to pancreatic cancer, wherein the mutation in KRAS is G12D, G12V, G12R, Q61H and/or Q61R, wherein the mutation in TP53 is R175H, R248W, G245S, R282W, R248Q and/or R273C, and wherein the mutation in U2AF1 is S34F. In certain embodiments, the method for treating a neoplastic disease in a subject in need thereof comprises administering an arenavirus particle to the subject, wherein the arenavirus particle encodes an antigenic fragment of a mutant KRAS, wherein the mutation in KRAS can be associated with or can be specific to pancreatic cancer, colorectal cancer or lung adenocarcinoma, and wherein the mutation in KRAS is KRAS is G12D, G12V, G12C, G12R, G13D, A146T, G12S, Q61H, G12A, and/or Q61R. In certain embodiments, the method for treating a neoplastic disease in a subject in need thereof comprises administering an arenavirus particle to the subject, wherein the arenavirus particle encodes an antigenic fragment of a mutant KRAS, mutant BRAF, mutant TP53, mutant FBXW7 and/or mutant PIK3CA, wherein the mutation in KRAS, BRAF, TP53, FBXW7 and/or PIK3CA can be associated with or can be specific to colorectal cancer or lung adenocarcinoma, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, and wherein the mutation in PIK3CA is E545K and/or H1047R. In certain embodiments, the method for treating a neoplastic disease in a subject in need thereof comprises administering an arenavirus particle to the subject, wherein the arenavirus particle encodes an antigenic fragment of a mutant KRAS, mutant BRAF, mutant PIK3CA, mutant EGFR, mutant TP53 and/or mutant U2AF1, wherein the mutation in KRAS, BRAF, PIK3CA, EGFR, TP53 and/or U2AF1 can be associated with or can be specific to pancreatic cancer, colorectal cancer or lung adenocarcinoma, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, and wherein the mutation in U2AF1 is S34F. In certain embodiments, the method for treating a neoplastic disease in a subject in need thereof comprises administering an arenavirus particle to the subject, wherein the arenavirus particle encodes an antigenic fragment of a mutant KRAS, mutant TP53, mutant U2AF1, mutant PIK3CA, mutant EGFR and/or mutant BRAF, wherein the mutation in KRAS, TP53, U2AF1, PIK3CA, EGFR and/or BRAF can be associated with or can be specific to lung adenocarcinoma, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E. In certain embodiments, the method for treating a neoplastic disease in a subject in need thereof comprises administering an arenavirus particle to the subject, wherein the arenavirus particle encodes an antigenic fragment of mutant KRAS, mutant AKT1, mutant BRAF, mutant HER2, mutant MEK1, mutant MET, mutant NRAS, mutant PIK3CA and/or mutant RET, wherein the mutations in KRAS, AKT1, BRAF, HER2, MEK1, MET, NRAS, PIK3CA and/or RET can be associated with or can be specific to NSCLC.

In certain embodiments, the method for treating a neoplastic disease in a subject in need thereof further comprises administering a second arenavirus particle to the subject, wherein the second arenavirus particle encodes an antigenic fragment of a mutant KRAS, a mutated cancer driver gene, or a tumor-associated antigen. In certain embodiments, the second arenavirus particle encodes an antigenic fragment of a mutant KRAS, wherein the mutation in KRAS can be associated with or can be specific to pancreatic cancer, and wherein the mutation in KRAS is KRAS is G12D, G12V, G12R, Q61H, Q61R, G12C, G12S, and/or G12A. In certain embodiments, the method for treating a neoplastic disease in a subject in need thereof further comprises administering a second arenavirus particle to the subject, wherein the second arenavirus particle encodes an antigenic fragment of a mutant KRAS, mutant TP53, and/or mutant U2AF1, wherein the mutation in KRAS, TP53 and/or U2AF1 can be associated with or can be specific to pancreatic cancer, wherein the mutation in KRAS is G12D, G12V, G12R, Q61H and/or Q61R, wherein the mutation in TP53 is R175H, R248W, G245S, R282W, R248Q and/or R273C, and wherein the mutation in U2AF1 is S34F. In certain embodiments, the method for treating a neoplastic disease in a subject in need thereof further comprises administering a second arenavirus particle to the subject, wherein the second arenavirus particle encodes an antigenic fragment of a mutant KRAS, wherein the mutation in KRAS can be associated with or can be specific to pancreatic cancer, colorectal cancer or lung adenocarcinoma, and wherein the mutation in KRAS is KRAS is G12D, G12V, G12C, G12R, G13D, A146T, G12S, Q61H, G12A, and/or Q61R. In certain embodiments, the method for treating a neoplastic disease in a subject in need thereof further comprises administering a second arenavirus particle to the subject, wherein the second arenavirus particle encodes an antigenic fragment of a mutant KRAS, mutant BRAF, mutant TP53, mutant FBXW7 and/or mutant PIK3CA, wherein the mutation in KRAS, BRAF, TP53, FBXW7 and/or PIK3CA can be associated with or can be specific to colorectal cancer or lung adenocarcinoma, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, and wherein the mutation in PIK3CA is E545K and/or H1047R. In certain embodiments, the method for treating a neoplastic disease in a subject in need thereof further comprises administering a second arenavirus particle to the subject, wherein the second arenavirus particle encodes an antigenic fragment of a mutant KRAS, mutant BRAF, mutant PIK3CA, mutant EGFR, mutant TP53 and/or mutant U2AF1, wherein the mutation in KRAS, BRAF, PIK3CA, EGFR, TP53 and/or U2AF1 can be associated with or can be specific to pancreatic cancer, colorectal cancer or lung adenocarcinoma, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, and wherein the mutation in U2AF1 is S34F. In certain embodiments, the method for treating a neoplastic disease in a subject in need thereof further comprises administering a second arenavirus particle to the subject, wherein the second arenavirus particle encodes an antigenic fragment of mutant KRAS, mutant TP53, mutant U2AF1, mutant PIK3CA, mutant EGFR and/or mutant BRAF, wherein the mutation in KRAS, TP53, U2AF1, PIK3CA, EGFR and/or BRAF can be associated with or can be specific to lung adenocarcinoma, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E. In certain embodiments, the method for treating a neoplastic disease in a subject in need thereof further comprises administering a second arenavirus particle to the subject, wherein the second arenavirus particle encodes an antigenic fragment of mutant KRAS, mutant AKT1, mutant BRAF, mutant HER2, mutant MEK1, mutant MET, mutant NRAS, mutant PIK3CA and/or mutant RET, wherein the mutations in AKT1, BRAF, HER2, MEK1, MET, NRAS, PIK3CA and/or RET can be associated with or can be specific to NSCLC.

### 5.2 Arenavirus Constructs

In certain embodiments, the arenavirus particles that can be engineered for the methods and compositions herein include the constructs listed below. In certain embodiments, the arenavirus construct is a non-replicating arenavirus construct as described in international patent application publication WO2009/083210 (which is incorporated herein in its entirety). See Section 5.3. In certain embodiments, the arenavirus construct is a replicating or a non-replicating tri-segmented arenavirus construct as described in international patent application publication WO2016/075250 and WO2021/089853 (both of which are incorporated herein in their entireties). See Sections 5.4 and 5.5.

Arenaviruses for use with the methods and compositions provided herein can be Old World viruses such as, for example, Lassa virus, Lymphocytic choriomeningitis virus (LCMV), Mobala virus, Mopeia virus, or Ippy virus, or New World viruses such as, for example, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, Allpahuayo virus (ALLV), or Whitewater Arroyo virus. Arenaviruses for use with the methods and compositions provided herein can be, for example, arenaviruses, mammarenaviruses, Old World mammarenaviruses, New World mammarenaviruses, New World mammarenaviruses of Clade A, New World mammarenaviruses of Clade B, New World mammarenaviruses of Clade C, or New World mammarenaviruses of Clade D. Arenaviruses for use with the methods and compositions provided herein can be a mammarenavirus including, but not limited to, Allpahuayo virus, Alxa virus, Junin virus, Bear Canyon virus, Sabia virus, Pichinde virus, Chapare virus, Lijiang virus, Cupixi virus, Flexal virus, Gairo virus, Guanarito virus, Ippy virus, Lassa virus, Latino virus, Loei River virus, Lujo virus, Luna virus, Luli virus, Lunk virus, lymphocytic choriomeningitis virus, Machupo virus, Mariental virus, Merino Walk virus, Mobala virus, Mopeia virus, Morogoro virus, Okahandja virus, Oliveros virus, Parana virus, Pirital virus, Apore virus, Ryukyu virus, Amapari virus, Solwezi virus, souris virus, Tacaribe virus, Tamiami virus, Wenzhou virus, Whitewater Arroyo virus, Big Brushy Tank virus, Catarina virus, Skinner Tank virus, Tonto Creek virus, or Xapuri virus. In certain embodiments, the arenavirus for use with the methods and compositions provided herein is an arenavirus of Clade A. In certain embodiments, the arenavirus for use with the methods and compositions provided herein is Pichinde virus.

### 5.3 Replication-Defective Arenavirus Particle

Exemplary replication-defective arenavirus particles are described, for example, in International Patent Application Publication WO2009/083210 (which is incorporated herein in its entirety). In certain embodiments, a replication-defective (e.g., replication-deficient) arenavirus particle with a nucleotide sequence encoding a mutant KRAS (see section 5.7), a mutated cancer driver gene (see section 5.8, e.g., a mutant TP53), or a tumor-associated antigen (see section 5.9) provided herein can be used with the methods and compositions provided herein. In specific embodiments, replication-defective arenavirus particles described herein are used with the methods and compositions provided herein in combination with replication-competent arenavirus particles described herein. In more specific embodiments, replication-defective arenavirus particles described herein are used with the methods and compositions provided herein in combination with replication-competent arenavirus particles described herein, wherein said replication-competent arenavirus particles are injected directly into a tumor in a subject.

In certain embodiments, provided herein is an arenavirus particle in which an ORF encoding GP, NP, Z protein, or L protein has been removed or functionally inactivated such that the resulting virus cannot produce further infectious progeny virus particles. An arenavirus particle comprising a genetically modified genome in which one or more ORFs has been deleted or functionally inactivated can be produced in complementing cells (i.e., cells that express the arenavirus ORF that has been deleted or functionally inactivated). The genetic material of the resulting arenavirus particle can be transferred upon infection of a host cell into the host cell, wherein the genetic material can be expressed and amplified.

In certain embodiments, such a heterologous nucleotide sequence can be polycistronic such that multiple polypeptides are ultimately produced from a single heterologous nucleotide sequence/transcript. This can be accomplished, e.g., using an internal ribosome entry site. In certain embodiments one such polypeptide can be a mutant KRAS. In certain embodiments, such a heterologous nucleotide sequence can encode an antigenic fragment of a mutant KRAS, a mutated cancer driver gene (e.g., a mutant TP53), or a tumor-associated antigen.

In certain embodiments, an ORF of the arenavirus is deleted or functionally inactivated and replaced with a nucleotide sequence encoding an antigenic fragment of a mutant KRAS, a mutated cancer driver gene (e.g., a mutant TP53), or a tumor-associated antigen as described herein. In a specific embodiment, the ORF that encodes the glycoprotein GP of the arenavirus is deleted or functionally inactivated. In certain embodiments, functional inactivation of a gene eliminates any translation product. In certain embodiments, functional inactivation refers to a genetic alteration that allows some translation, the translation product, however, is no longer functional and cannot replace the wild-type protein.

In certain embodiments, at least one of the four arenaviral ORFs encoding GP, NP, Z protein, and L protein is removed and replaced with a nucleotide sequence encoding an antigenic fragment of a mutant KRAS, a mutated cancer driver gene (e.g., a mutant TP53), or a tumor-associated antigen provided herein. In another embodiment, at least one ORF, at least two ORFs, at least three ORFs, or at least four ORFs encoding GP, NP, Z protein and L protein can be removed and replaced with a nucleotide sequence encoding an antigenic fragment of a mutant KRAS, a mutated cancer driver gene (e.g., a mutant TP53), or a tumor-associated antigen provided herein. In specific embodiments, only one of the four ORFs encoding GP, NP, Z protein, and L protein is removed and replaced with a nucleotide sequence encoding an antigenic fragment of a mutant KRAS, a mutated cancer driver gene (e.g., a mutant TP53), or a tumor-associated antigen provided herein. In more specific embodiments, the ORF that encodes GP of the arenavirus genomic segment is removed. In another specific embodiment, the ORF that encodes NP of the arenavirus genomic segment is removed. In more specific embodiments, the ORF that encodes the Z protein of the arenavirus genomic segment is removed. In yet another specific embodiment, the ORF encoding the L protein of the arenavirus genomic segment is removed.

Thus, in certain embodiments, the arenavirus particle provided herein comprises a genomic segment in which (i) an ORF encoding GP, NP, Z protein, or L protein is removed; and (ii) the ORF that is removed is replaced with a nucleotide sequence encoding a mutant KRAS (see section 5.7), a mutated cancer driver gene (see section 5.8, e.g., a mutant TP53), or a tumor-associated antigen (see section 5.9) provided herein.

In certain embodiments, the growth in complementing cells and infectivity of the arenavirus particle is not affected by the nucleotide sequence encoding an antigenic fragment of a mutant KRAS (see section 5.7), a mutated cancer driver gene (see section 5.8, e.g., a mutant TP53), or a tumor-associated antigen (see section 5.9) provided herein.

In certain embodiments, an arenavirus particle or arenavirus genomic segment provided herein comprising a nucleotide sequence encoding an antigenic fragment of mutant KRAS (see section 5.7) as provided herein can further comprise at least one nucleotide sequence encoding at least one antigenic fragment of a mutated cancer driver gene, or a tumor-associated antigen. In certain embodiments, the mutated cancer driver gene is mutant AKT1, mutant BRAF, mutant HER2, mutant MEK1, mutant MET, mutant NRAS, mutant PIK3CA, mutant RET, mutant APC, mutant TP53, mutant EGFR, mutant FBXW7, mutant SMAD4, mutant GNAS, mutant ERBB2, mutant ERBB3, mutant CDKN2A, mutant CTNNB1, and/or mutant U2AF1(see section 5.8). In certain embodiments, the tumor-associated antigen is derived from the BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family (see section 5.9).

In certain embodiments, the arenavirus particle as described herein is suitable for use as a vaccine, immunotherapy, or pharmaceutical composition and methods of using such arenavirus particle in the treatment of neoplastic diseases, for example, cancer, is provided. More detailed description of the methods of using the arenavirus particle described herein is provided in section (see section 5.10)

5.4 Tri-Segmented Arenavirus Particle with Rearrangements of their ORFs

Exemplary tri-segmented arenavirus particles are described, for example, in International Patent Application Publication WO 2016/075250 and WO 2017/198726, which are incorporated by reference herein in their entireties.

In certain embodiments, tri-segmented arenavirus particles with rearrangements of their ORFs comprising a nucleotide sequence encoding an antigenic fragment of a mutant KRAS (see section 5.7), a mutated cancer driver gene (see section 5.8, e.g., a mutant TP53), or a tumor-associated antigen (see section 5.9) provided herein can be used with the methods and compositions provided herein. In one aspect, provided herein is a tri-segmented arenavirus particle comprising one L segment and two S segments or two L segments and one S segment. See section 5.6(b) for manufacturing methods. In certain embodiments, propagation of the tri-segmented arenavirus particle does not result in a replication competent bi-segmented arenavirus particle. In other words, the tri-segmented viruses provided herein are genetically stable. More specifically, in certain embodiments, two of the genomic segments (e.g., the two S segments or the two L segments, respectively) cannot recombine in a way to yield a single viral segment that could replace the two parent segments. In certain embodiments, inter-segmental recombination of two of the genomic segments (e.g., the two S segments or the two L segments, respectively), uniting two arenavirus ORFs on only one instead of two separate segments, abrogates viral promoter activity. In specific embodiments, the genome of the tri-segmented arenavirus particle comprises an arenaviral ORF in a position other than the wild-type position of the ORF and a nucleotide sequence encoding an antigenic fragment of a mutant KRAS (see section 5.7), a mutated cancer driver gene (see section 5.8, e.g., a mutant TP53), or a tumor-associated antigen (see section 5.9) provided herein. In yet another specific embodiment, the genome of the tri-segmented arenavirus particle comprises all four arenavirus ORFs. Thus, in certain embodiments, the tri-segmented arenavirus particle is replication competent and infectious.

In certain embodiments, the genome of such a tri-segmented arenavirus particle (see section 5.6(b)) that is replication competent and infectious has two available positions for inclusion of heterologous nucleotide sequences. These positions can be used for integration of heterologous nucleotide sequences, e.g., as set forth in Table 1 below. In certain embodiments, each such heterologous nucleotide sequence can be transcribed into a single transcript. In certain embodiments, each such heterologous nucleotide sequence encodes a polypeptide. In certain embodiments, such a heterologous nucleotide sequence can be polycistronic such that multiple polypeptides are ultimately produced from a single heterologous nucleotide sequence/transcript. This can be accomplished, e.g., by using an internal ribosome entry site. In certain embodiments one such polypeptide can be a mutant KRAS (see section 5.7), a mutated cancer driver gene (see section 5.8, e.g., a mutant TP53), or a tumor-associated antigen (see section 5.9). In certain embodiments, the heterologous nucleotide sequences at the two available positions encode both a mutant KRAS. In other embodiments, the heterologous nucleotide sequence at one of the two available positions encodes an antigenic fragment of a mutant KRAS, and the heterologous nucleotide sequence at the other of the two available positions encodes an antigenic fragment of a mutated cancer driver gene (e.g., a mutant TP53), or a tumor-associated antigen.

In certain embodiments, tri-segmented arenavirus particles (see section 5.6(b)) with rearrangements of their ORFs comprising a nucleotide sequence that does not encode a foreign antigen can be used with the methods and compositions provided herein. In specific embodiments, the tri-segmented arenavirus particle comprises an ORF in a position other than the wild-type position of the ORF. In yet another specific embodiment, the tri-segmented arenavirus particle comprises all four arenavirus ORFs. Thus, in certain embodiments, the tri-segmented arenavirus particle is replication competent and infectious.

In certain embodiments, the ORF encoding GP, NP, Z protein, or L protein of the tri-segmented arenavirus particle (see section 5.6(b)) described herein can be under the control of an arenavirus genomic 3′ UTR or an arenavirus genomic 5′ UTR. In more specific embodiments, the arenavirus genomic 3′ UTR is the 3′ UTR of an arenavirus S segment. In another specific embodiment, the arenavirus genomic 3′ UTR is the 3′ UTR of an arenavirus L segment. In more specific embodiments, the arenavirus genomic 5′ UTR is the 5′ UTR of an arenavirus S segment. In other specific embodiments, the arenavirus genomic 5′ UTR is the 5′ UTR of an arenavirus L segment.

In other embodiments, the ORF encoding GP, NP, Z protein, or L protein of the tri-segmented arenavirus particle (see section 5.6(b) for manufacturing methods) described herein can be under the control of the arenavirus conserved terminal sequence element (the 5′-and 3-terminal 19-20-nt regions) (see e.g., Perez & de la Torre, 2003, J Virol. 77(2): 1184-1194).

In certain embodiments, the ORF encoding GP, NP, Z protein or L protein of the tri-segmented arenavirus particle (see section 5.6(b) for manufacturing methods) can be under the control of the promoter element of the 5′ UTR (see e.g., Albarino et al., 2011, J Virol., 85(8):4020-4). In another embodiment, the ORF encoding GP, NP. Z protein, L protein of the tri-segmented arenavirus particle can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment(s) or the L segment(s). In another specific embodiment, the promoter element of the 3′ UTR is the 3′ UTR the promoter element of the S segment(s) or the L segment(s).

In certain embodiments, the ORF that encodes GP, NP, Z protein or L protein of the tri-segmented arenavirus particle can be under the control of a truncated arenavirus 3′ UTR or a truncated arenavirus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is derived from the 3′ UTR of the arenavirus S segment or L segment. In more specific embodiments, the truncated 5′ UTR is derived from the 5′ UTR of the arenavirus S segment(s) or L segment(s).

Also provided herein, is a cDNA of the genome of the tri-segmented arenavirus particle comprising a nucleotide sequence encoding an antigenic fragment of a mutant KRAS (see section 5.7), a mutated cancer driver gene (see section 5.8, e.g., a mutant TP53), or a tumor-associated antigen (see section 5.9) provided herein. In more specific embodiments, provided herein is a DNA nucleic acid or a set of DNA nucleic acids encoding the genome of a tri-segmented arenavirus particle as set forth in Table 1.

In certain embodiments, the nucleic acids encoding the genome of the tri-segmented arenavirus genome (see section 5.6(b)) are part of or incorporated into one or more DNA expression vectors. In a specific embodiment, nucleic acids encoding the genome of the tri-segmented arenavirus particle are part of or incorporated into one or more DNA expression vectors that facilitate production of a tri-segmented arenavirus particle as described herein. In another embodiment, a cDNA described herein can be incorporated into a plasmid. Techniques for the production of a cDNA and routine and conventional techniques of molecular biology and DNA manipulation and production, including any cloning technique known to the skilled artisan can be used. Such techniques are well known and are available to the skilled artisan in laboratory manuals such as, Sambrook and Russell, Molecular Cloning: A laboratory Manual, 3^{rd} edition, Cold Spring Harbor Laboratory N.Y. (2001).

Provided herein are cell lines, cultures and methods of culturing cells transfected with nucleic acids, vectors, and compositions provided herein.

In specific embodiments, the arenavirus particle described herein is attenuated. In a particular embodiment, the tri-segmented arenavirus particle is attenuated such that the virus remains, at least partially, replication-competent and can replicate in vivo, but can only generate low viral loads resulting in subclinical levels of infection that are non-pathogenic. Such attenuated viruses can be used as an immunogenic composition.

In certain embodiments, the tri-segmented arenavirus particle has the same tropism as the bi-segmented arenavirus particle from which the tri-segmented virus was derived.

Also provided herein, are pharmaceutical compositions that comprise the tri-segmented arenavirus particle as described herein.

### (a) Tri-Segmented Arenavirus Particle Comprising One L Segment and Two S Segments

In one aspect, provided herein is a tri-segmented arenavirus particle comprising one L segment and two S segments. In certain embodiments, propagation of the tri-segmented arenavirus particle comprising one L segment and two S segments does not result in a replication-competent bi-segmented viral particle. In specific embodiments, propagation of the tri-segmented arenavirus particle comprising one L segment and two S segments does not result in a replication-competent bi-segmented viral particle after at least 10 days, at least 20 days, at least 30 days, at least 40 days, at least 50 days, at least 60 days, at least 70 days, at least 80 days, at least 90 days, or at least 100 days of persistent infection in mice lacking type I interferon receptor, type II interferon receptor and recombination activating gene (RAG1), and having been infected with 10⁴ PFU of the tri-segmented arenavirus particle (see Section 5.12(m)). In other embodiments, propagation of the tri-segmented arenavirus particle comprising one L segment and two S segments does not result in a replication-competent bi-segmented viral particle after at least 10 passages, at least 20 passages, at least 30 passages, at least 40 passages, or at least 50 passages.

In particular, the genome of the tri-segmented arenavirus particle comprises one L segment and two S segments, in which a nucleotide sequence encoding a mutant KRAS (see section 5.7), a mutated cancer driver gene (see section 5.8, e.g., a mutant TP53), or a tumor-associated antigen (see section 5.9) provided herein is inserted into one position on each S segment. More specifically, one S segment encodes the arenaviral GP and a mutant KRAS, a mutated cancer driver gene (e.g., a mutant TP53), or a tumor-associated antigen thereof, respectively. The other S segment encodes the arenaviral NP and a mutant KRAS, a mutated cancer driver gene (e.g., a mutant TP53), or a tumor-associated antigen. The L segment encodes the arenaviral L protein and Z protein. All segments are flanked by the respective 5′ and 3′ UTRs.

More specifically, provided herein is an arenavirus comprising:
(i) a first S-Segment comprising the nucleotide sequence encoding the antigenic fragment(s) (such as a mutant KRAS (see section 5.7), a mutated cancer driver gene (see section 5.8, e.g., a mutant TP53), or a tumor-associated antigen (see section 5.9)) under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence encoding the antigenic fragment(s) (such as a mutant KRAS (see section 5.7), a mutated cancer driver gene (see section 5.8, e.g., a mutant TP53), or a tumor-associated antigen (see section 5.9)) under control of an arenavirus genomic 5′ UTR and an ORF encoding the arenaviral glycoprotein (“GP”) under control of an arenavirus genomic 3′ UTR; and
(iii) an L-Segment.

In certain embodiments, inter-segmental recombination of the two S segments of the tri-segmented arenavirus particle, provided herein, that unities the two arenaviral ORFs on one instead of two separate segments results in a nonfunctional promoter (i.e., a genomic segment of the structure: 5′ UTR-----------5′ UTR or a 3′ UTR------------3′ UTR), wherein each UTR forming one end of the genome is an inverted repeat sequence of the other end of the same genome.

In certain embodiments, the tri-segmented arenavirus particle comprising one L segment and two S segments has been engineered to carry an arenavirus ORF in a position other than the wild-type position of the ORF and a nucleotide sequence encoding a mutant KRAS (see section 5.7), a mutated cancer driver gene (see section 5.8, e.g., a mutant TP53), or a tumor-associated antigen (see section 5.9) provided herein. In other embodiments, the tri-segmented arenavirus particle comprising one L segment and two S segments has been engineered to carry two arenavirus ORFs, or three arenavirus ORFs, or four arenavirus ORFs, or five arenavirus ORFs, or six arenavirus ORFs in a position other than the respective wild-type positions. In specific embodiments, the tri-segmented arenavirus particle comprising one L segment and two S segments comprises a full complement of all four arenavirus ORFs. Thus, in some embodiments, the tri-segmented arenavirus particle is an infectious and replication competent tri-segmented arenavirus particle. In specific embodiments, the two S segments of the tri-segmented arenavirus particle have been engineered to carry one of their ORFs in a position other than the wild-type position. In more specific embodiments, the two S segments comprise a full complement of the S segment ORFs. In certain specific embodiments, the L segment has been engineered to carry an ORF in a position other than the wild-type position or the L segment can be the wild-type genomic segment.

In certain embodiments, one of the two S segments can be:
(i) an arenavirus S segment, wherein the ORF encoding the Z protein is under control of an arenavirus 5′ UTR;
(ii) an arenavirus S segment, wherein the ORF encoding the L protein is under control of an arenavirus 5′ UTR;
(iii) an arenavirus S segment, wherein the ORF encoding the NP is under control of an arenavirus 5′ UTR;
(iv) an arenavirus S segment, wherein the ORF encoding the GP is under control of an arenavirus 3′ UTR;
(v) an arenavirus S segment, wherein the ORF encoding the L protein is under control of an arenavirus 3′ UTR; and
(vi) an arenavirus S segment, wherein the ORF encoding the Z protein is under control of an arenavirus 3′ UTR.

In certain embodiments, the tri-segmented arenavirus particle comprising one L segment and two S segments can comprise a duplicate arenaviral ORF (i.e., two wild-type ORFs encoding e.g., GP or NP). In specific embodiments, the tri-segmented arenavirus particle comprising one L segment and two S segments can comprise one duplicate ORF (e.g., (GP, GP)) or two duplicate ORFs (e.g., (GP, GP) and (NP, NP)).

Table 1, below, is an illustration of the genome organization of a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 1
Tri-segmented arenavirus particle comprising one L segment and
two S segments Position 1 is under the control of an arenavirus S
segment 5′ UTR; Position 2 is under the control of an arenavirus S
segment 3′ UTR; Position 3 is under the control of an arenavirus S
segment 5′ UTR; Position 4 under the control of an arenavirus S
segment 3′ UTR; Position 5 is under the control of an arenavirus L
segment 5′ UTR; Position 6 is under the control of an
arenavirus L segment 3′ UTR.

| Position 1 | Position 2 | Position 3 | Position 4 | Position 5 | Position 6 |
|---|---|---|---|---|---|
| *ORF | GP | *ORF | NP | Z | L |
| *ORF | GP | *ORF | NP | L | Z |
| *ORF | NP | *ORF | GP | Z | L |
| *ORF | NP | *ORF | GP | L | Z |
| *ORF | NP | *ORF | Z | L | GP |
| *ORF | NP | Z | GP | *ORF | L |
| *ORF | NP | Z | GP | L | *ORF |
| *ORF | NP | *ORF | L | Z | GP |
| *ORF | L | *ORF | NP | Z | GP |
| *ORF | L | Z | NP | *ORF | GP |
| *ORF | L | *ORF | GP | Z | NP |
| *ORF | L | Z | GP | *ORF | NP |
| *ORF | Z | L | NP | *ORF | GP |
| *ORF | Z | *ORF | GP | L | NP |
| *ORF | Z | L | GP | *ORF | NP |
| L | GP | *ORF | NP | *ORF | Z |
| L | GP | *ORF | NP | Z | *ORF |
| L | GP | *ORF | Z | *ORF | NP |
| L | GP | Z | *ORF | *ORF | NP |
| L | *ORF | Z | GP | *ORF | NP |
| L | GP | Z | NP | *ORF | *ORF |
| L | *ORF | Z | NP | *ORF | GP |
| L | NP | *ORF | GP | Z | *ORF |
| L | NP | *ORF | Z | *ORF | GP |
| L | NP | Z | *ORF | GP | *ORF |
| L | *ORF | Z | *ORF | GP | NP |
| L | NP | Z | GP | *ORF | *ORF |
| L | NP | Z | *ORF | *ORF | GP |
| L | Z | *ORF | GP | *ORF | NP |
| L | Z | *ORF | NP | *ORF | GP |
| Z | GP | *ORF | NP | *ORF | L |
| Z | GP | *ORF | NP | L | *ORF |
| Z | GP | *ORF | L | *ORF | NP |
| Z | GP | L | *ORF | *ORF | NP |
| Z | *ORF | L | GP | *ORF | NP |
| Z | GP | L | NP | *ORF | *ORF |
| Z | *ORF | L | NP | *ORF | GP |
| Z | NP | *ORF | GP | *ORF | L |
| Z | NP | *ORF | L | *ORF | GP |
| Z | NP | L | GP | *ORF | *ORF |
| Z | NP | L | *ORF | *ORF | GP |
| Z | NP | *ORF | GP | L | *ORF |
| Z | L | *ORF | GP | *ORF | NP |

*ORF indicates that a nucleotide sequence encoding an antigenic fragment of a mutant KRAS (see section 5.7), a mutated cancer driver gene (see section 5.8, e.g., a mutant TP53), or a tumor-associated antigen (see section 5.9) provided herein has been inserted.

In certain embodiments, the IGR between position one and position two can be an arenavirus S segment or L segment IGR; the IGR between position three and position four can be an arenavirus S segment or L segment IGR; and the IGR between position five and position six can be an arenavirus L segment IGR. In a specific embodiment, the IGR between position one and position two can be an arenavirus S segment IGR; the IGR between position three and position four can be an arenavirus S segment IGR; and the IGR between position five and position six can be an arenavirus L segment IGR. In certain embodiments, other combinations are also possible. For example, a tri-segmented arenavirus particle comprising one L segment and two S segments, wherein intersegmental recombination of the two S segments in the tri-segmented arenavirus genome does not result in a replication-competent bi-segmented viral particle and abrogates arenaviral promoter activity (i.e., the resulting recombined S segment is made up of two 5′ UTRs instead of a 3′ UTR and a 5′ UTR).

In certain embodiments, intersegmental recombination of an S segment and an L segment in the tri-segmented arenavirus particle comprising one L segment and two S segments, restores a functional segment with two viral genes on only one segment instead of two separate segments. In other embodiments, intersegmental recombination of an S segment and an L segment in the tri-segmented arenavirus particle comprising one L segment and two S segments does not result in a replication-competent bi-segmented viral particle.

In certain embodiments, one of skill in the art could construct an arenavirus genome with an organization as illustrated in Table 1 and as described herein, and then use an assay as described in Section 5.12 to determine whether the tri-segmented arenavirus particle is genetically stable, i.e., does not result in a replication-competent bi-segmented viral particle as discussed herein.

### 5.5 Split Arenavirus Vector Particles

Arenaviruses can also be engineered in the way described in international patent application publication WO 2021/089853 and U.S. Provisional Application No. 63/188,317 filed May 13, 2021 (which are incorporated herein in its entirety). This technology is also called “split” vector technology. Similar to the tri-segmented viruses described above, the technology described in WO 2021/089853 can be used to generate tri-segmented viruses with two open positions for heterologous nucleotide sequences. Such a heterologous nucleotide sequence can encode a polypeptide. In certain embodiments one such polyeptide can be a mutant KRAS (see section 5.7), a mutated cancer driver gene (see section 5.8, e.g., a mutant TP53), or a tumor-associated antigen (see section 5.9).

Briefly, such a “split” arenavirus particle is engineered such that an arenaviral ORF is separated over two or more mRNA transcripts. In certain embodiments, provided herein is an arenavirus genomic or antigenomic segment engineered such that the transcription thereof results in one or more mRNA transcripts comprising a nucleotide sequence encoding a functional fragment of arenavirus GP, NP, L protein or Z protein.

In certain embodiments, the ORF encoding the arenavirus GP is separated (or split) over two mRNA transcripts and over two positions of the arenavirus genome, respectively. For example, the arenavirus GP signal peptide or a functional fragment thereof can be expressed from a first mRNA transcript (e.g., viral mRNA transcript) and arenavirus GP1 and GP2 subunits are expressed from a second mRNA transcript (e.g., viral mRNA transcript). In certain embodiments, the first mRNA transcript is under control of an arenavirus 3′ genomic UTR. In certain embodiments, the second mRNA transcript further encodes a heterologous non-arenaviral signal peptide (such as the signal peptide of the vesicular stomatitis virus serotype Indiana glycoprotein). In certain embodiments, the first mRNA transcript further comprises a nucleotide sequence encoding a heterologous non-arenaviral polypeptide, namely a mutant KRAS (see section 5.7), a mutated cancer driver gene (see section 5.8, e.g., a mutant TP53), or a tumor-associated antigen (see section 5.9).

In certain embodiments, the genomic organization of such a “split” arenavirus vector is as follows:
First S segment: arenavirus GP1 and GP2 subunits fused to a heterologous signal peptide under control of an arenavirus genomic 5′ UTR; fusion of arenavirus GP signal peptide and a nucleotide sequence encoding the antigenic fragment(s) under control of an arenavirus genomic 3′ UTR.
Second S segment: a nucleotide sequence encoding the antigenic fragment(s) under control of an arenavirus genomic 5′ UTR and an ORF encoding the nucleoprotein (“NP”) under control of an arenavirus genomic 3′ UTR.
L segment: the arenavirus Z protein under control of an arenavirus genomic 5′ UTR; the arenavirus L protein under control of an arenavirus genomic 3′ UTR.

In certain embodiments, the nucleotide sequence encoding the antigenic fragment(s) on the first S-Segment is different from the nucleotide sequence encoding the antigenic fragment(s) on the second S-Segment. In certain embodiments, the nucleotide sequence encoding the antigenic fragment(s) on the first S-Segment is the same as the nucleotide sequence encoding the antigenic fragment(s) on the second S-Segment.

In certain embodiments, the antigenic fragment(s) encoded on the first S-Segment is/are different from the antigenic fragment(s) encoded on the second S-Segment. In certain embodiments, the antigenic fragment(s) encoded on the first S-Segment is/are the same as the antigenic fragment(s) encoded on the second S-Segment. In certain embodiments, the antigenic fragments encoded on the first S-Segment are the same as the antigenic fragments encoded on the second S-Segment but are fused to each other in a different order from the order in which the antigenic fragments encoded on the second S-Segment are fused to each other.

### 5.6 Generation of Arenavirus Particles

Generally, arenavirus particles for use in the methods and compositions provided herein can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the arenavirus particles provided herein, these techniques can be applied as described below. The genome of the viruses can be modified as described herein.

### (a) Generation of Replication-Deficient Arenavirus Particles

An arenavirus particle engineered to comprise a genome with the ability to amplify and express its genetic information in infected cells but unable to produce further infectious progeny particles in normal, non-complementing cells, wherein one arenavirus open reading frame is removed and replaced by a nucleotide sequence encoding a mutant KRAS (see section 5.7), a mutated cancer driver gene (see section 5.8, e.g., a mutant TP53), or a tumor-associated antigen (see section 5.9) can be recombinantly produced by any reverse genetic technique known to one skilled in the art.

In certain embodiments, the method of generating the infectious, replication-deficient arenavirus particle comprises (i) transfecting into a complementing host cell (which expresses the open reading frame that is deleted or functionally inactivated of the genomic segment) the cDNA of the first arenavirus genomic segment; (ii) transfecting into a host cell the cDNA of the second arenavirus genomic segment; (iii) transfecting into a host cell plasmids expressing the arenavirus' minimal trans-acting factors NP and L; (iv) maintaining the host cell under conditions suitable for virus formation; and (v) harvesting the arenavirus particle. In certain more specific embodiments, the cDNA is comprised in a plasmid.

Once generated from cDNA, the infectious, replication-deficient arenaviruses can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the EF1 alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

In certain embodiments, the complementing host cells are kept in culture and are transfected with one or more plasmid(s). The plasmid(s) encode the arenavirus genomic segment(s) of the arenavirus particle to be generated under control of a polymerase I promoter and terminator.

Plasmids that can be used for the generation of the arenavirus particle can include: i) a plasmid encoding the S genomic segment e.g., pol-I S, ii) a plasmid encoding the L genomic segment e.g., pol-I L. In certain embodiments, the plasmid encoding an arenavirus polymerase that direct intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, poll and polII promoters from one plasmid.

In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

For recovering the arenavirus particle described herein, the following procedures are envisaged. First day: complementing cells, are transfected with a mixture of the plasmids, as described above. For this one can exploit any commonly used strategies such as calcium-phosphate, liposome-based protocols or electroporation.

3-5 days later: The cell suspension (i.e., cells and medium) is harvested. Arenavirus particles present in the medium are cleared from cells and debris by centrifugation and the supernatant (i.e., the arenavirus vector preparation) is aliquoted and stored at 4° C., −20° C., or −80° C. The arenavirus vector preparation's infectious titer is assessed by an immunofocus assay. Alternatively, the transfected cells and supernatant may be passaged to a larger vessel on day 3-5 after transfection, and vectors are harvested up to five days after passage as described before.

(b) Generation of a Tri-Segmented, replication-competent Arenavirus Particle

A tri-segmented arenavirus particle comprising a genomic segment that has been engineered to carry a viral ORF in a position other than the wild-type position of the ORF and further comprising a nucleotide sequence encoding a mutant KRAS (see section 5.7), a mutated cancer driver gene (see section 5.8, e.g., a mutant TP53), or a tumor-associated antigen (see section 5.9) can be recombinantly produced by reverse genetic techniques known in the art, for example as described by Emonet et al., 2008, PNAS, 106(9):3473-3478; Popkin et al., 2011, J. Virol., 85 (15):7928-7932, which are incorporated by reference herein. Both vector systems described in sections 5.4 and 5.5 can be generated using the methods described in this section.

In certain embodiments, the method of generating the tri-segmented arenavirus particle comprises (i) transfecting into a host cell the cDNAs of the one arenavirus L segment and two arenavirus S segments or two arenavirus L segments and one arenavirus S segment; (ii) transfecting into a host cell plasmids expressing the arenavirus' minimal trans-acting factors NP and L protein; (iii) maintaining the host cell under conditions suitable for virus formation; and (iv) harvesting the arenavirus particle. In certain more specific embodiments, the cDNA of the arenavirus S and L segments is comprised in a plasmid.

Once generated from cDNA, the tri-segmented arenavirus particle (i.e., infectious and replication competent) can be propagated. In certain embodiments tri-segmented arenavirus particles can be propagated in any host cell that allows the virus to grow to titers that permit the uses of the virus as described herein. In one embodiment, the host cell allows the tri-segmented arenavirus particle to grow to titers comparable to those determined for the corresponding wild-type.

In certain embodiments, the tri-segmented arenavirus particle may be propagated in host cells. Specific examples of host cells that can be used include BHK-21, HEK 293, VERO or other. In a specific embodiment, the tri-segmented arenavirus particle may be propagated in a cell line.

In certain embodiments, the host cells are kept in culture and are transfected with one or more plasmid(s). The plasmid(s) encode the arenavirus genomic segment(s) of the arenavirus particle to be generated under control of a polymerase I promoter and terminator.

In specific embodiments, the host cells are kept in culture and are transfected with one or more plasmid(s). The plasmid(s) encode the viral protein(s) to be generated under control of one or more expression cassettes suitable for expression in mammalian cells, e.g., consisting of a polymerase II promoter and terminator.

Plasmids that can be used for generating the tri-segmented arenavirus comprising one L segment and two S segments can include: i) two plasmids each encoding the S genome segment e.g., pol-I S, ii) a plasmid encoding the L genome segment e.g., pol-I L. Plasmids needed for the tri-segmented arenavirus comprising two L segments and one S segments are: i) two plasmids each encoding the L genome segment e.g., pol-L, ii) a plasmid encoding the S genome segment e.g., pol-I S.

In certain embodiments, a plasmid encoding an arenavirus polymerase that directs intracellular synthesis of the viral L and S segments can be incorporated into the transfection mixture. For example, a plasmid encoding the L protein and a plasmid encoding NP (pC-L and pC-NP, respectively) can be used. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In addition, the plasmid(s) features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of host cells with a plasmid(s) can be performed using any of the commonly used strategies such as calcium-phosphate, liposome-based protocols or electroporation..

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the arenavirus genomic segments can be the same, i.e., the genome sequence and transacting factors can be transcribed by T7, poll and polII promoters from one plasmid.

In other embodiments, transcription of the arenavirus genomic segment is performed using a bi-directional expression cassette (see e.g., Ortiz-Riano et al., 2013, J Gen Virol., 94(Pt 6): 1175-1188). In more specific embodiments the bi-directional expression cassette comprises both a polymerase I and a polymerase II promoter reading from opposite sides into the two termini of the inserted arenavirus genomic segment, respectively.

In other embodiments, transcription of the cDNA of the arenavirus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

For recovering the tri-segmented arenavirus vector, the following procedures are envisaged. First day: cells, are transfected with a mixture of the plasmids, as described above. For this one can exploit any commonly used strategies such as calcium-phosphate, liposome-based protocols or electroporation.

3-5 days later: The cell suspension (i.e., cells and medium) is harvested. Arenavirus particles present in the medium are cleared from cells and debris by centrifugation and the supernatant (i.e., the arenavirus vector preparation) is aliquoted and stored at 4° C., −20° C., or −80° C. The arenavirus vector preparation's infectious titer is assessed by an immunofocus assay. Alternatively, the transfected cells and supernatant may be passaged to a larger vessel on day 3-5 after transfection, and vectors are harvested up to five days after passage as described before.

In certain embodiments, expression of a nucleotide sequence encoding a mutant KRAS (see section 5.7), a mutated cancer driver gene (see section 5.8, e.g., a mutant TP53), or a tumor-associated antigen (see section 5.9) is provided, wherein a plasmid encoding the genomic segment is modified to incorporate a nucleotide sequence encoding a mutant KRAS, a mutated cancer driver gene (e.g., a mutant TP53), or a tumor-associated antigen. The nucleotide sequence encoding a mutant KRAS, a mutated cancer driver gene (e.g., a mutant TP53), or a tumor-associated antigen can be incorporated into the plasmid using restriction enzymes.

### 5.7 Mutant KRAS

In the context of this application, “mutant KRAS” means a polypeptide encoded by a mutated KRAS gene.

In certain embodiments, arenavirus particles with a nucleotide sequence encoding an antigenic fragment of a mutant KRAS provided herein can be used with the methods and compositions provided herein. In certain embodiments, arenavirus particles with a nucleotide sequence encoding an antigenic fragment of a mutant KRAS that further comprise a nucleotide sequence encoding one or more antigenic fragment(s) of mutated cancer driver gene(s) (e.g., a mutant TP53) or tumor-associated antigen(s) (i.e., the same arenavirus particle comprising different nucleotide sequences) are provided herein and can be used with the methods and compositions provided herein. In certain embodiments, arenavirus particles with a nucleotide sequence encoding an antigenic fragment of a mutant KRAS and one or more antigenic fragment(s) of mutated cancer driver gene(s) (e.g., a mutant TP53) or tumor-associated antigen(s) (i.e., the same nucleotide sequence encoding different antigenic fragments) are provided herein and can be used with the methods and compositions provided herein. In certain embodiments, the mutation in KRAS is at amino acid position G12, G13, A18, A59, Q61, K117, A146, or D119 of KRAS. In certain embodiments, the mutation in KRAS is A18D, A59E, A59G, A59P, A59T, A59S, A59V, A146P, A146S, A146T, A146V, D119N, G12A, G12C, G12D, G12F, G12L, G12R, G12S, G12V, G13A, G13C, G13D, G13E, G13R, G13S, G13V, K117N, Q61E, Q61H, Q61K, Q61L, Q61P, Q61R or a combination thereof. In certain embodiments, the mutation in KRAS is G12A, G12C, G12D, G12R, G12S, G12V, G13D, Q61H, Q61R, A146T or a combination thereof. In certain embodiments, the mutation in KRAS is G13D, G12V, G12C, G12D, G12R or a combination thereof. In a more specific embodiment, the arenavirus genome comprises a nucleotide sequence encoding from N- to C-terminus fragments of mutant KRAS comprising the mutations G13D, G12V, G12C, G12D, and G12R, respectively. In other more specific embodiments, the arenavirus genome comprises a nucleotide sequence encoding fragments of mutant KRAS comprising the mutations G13D, G12V, G12C, G12D, and G12R in any possible order.

In certain embodiments, the nucleotide sequence is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of SEQ ID NO:20.

In certain embodiments, the nucleotide sequence encodes an expression product whose amino acid sequence is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:19.

In certain embodiments, the nucleotide sequence encodes a fragment of mutant KRAS, wherein the fragment is 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, 26 amino acids in length, 27 amino acids in length, 28 amino acids in length, 29 amino acids in length, or 30 amino acids in length; and wherein the fragment comprises the mutation of the mutant KRAS. In a more specific embodiment, the nucleotide sequence encodes a fragment of mutant KRAS, wherein the fragment is 18 amino acids in length.

In certain embodiments, the region flanking the mutation at the N-terminus of the antigenic fragment is 0 amino acids in length, 1 amino acid in length, 2 amino acids in length, 3 amino acids in length, 4 amino acids in length, 5 amino acids in length, 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, or 20 amino acids in length. In a more specific embodiment, the region flanking the mutation at the N-terminus of the antigenic fragment is 8 amino acids in length. In another more specific embodiment, the region flanking the mutation at the N-terminus of the antigenic fragment is 9 amino acids in length. In certain embodiments, the region flanking the mutation at the C-terminus of the antigenic fragment is 0 amino acids in length, 1 amino acid in length, 2 amino acids in length, 3 amino acids in length, 4 amino acids in length, 5 amino acids in length, 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, or 20 amino acids in length. In a more specific embodiment, the region flanking the mutation at the C-terminus of the antigenic fragment is 8 amino acids in length. In another more specific embodiment, the region flanking the mutation at the C-terminus of the antigenic fragment is 9 amino acids in length.

In certain embodiments, the arenavirus particle comprises an arenavirus genome comprising a nucleotide sequence encoding two antigenic fragments of mutant KRAS, three antigenic fragments of mutant KRAS, four antigenic fragments of mutant KRAS, five antigenic fragments of mutant KRAS, six antigenic fragments of mutant KRAS, seven antigenic fragments of mutant KRAS, eight antigenic fragments of mutant KRAS, nine antigenic fragments of mutant KRAS, ten antigenic fragments of mutant KRAS, eleven antigenic fragments of mutant KRAS, twelve antigenic fragments of mutant KRAS, thirteen antigenic fragments of mutant KRAS, fourteen antigenic fragments of mutant KRAS, fifteen antigenic fragments of mutant KRAS, sixteen antigenic fragments of mutant KRAS, seventeen antigenic fragments of mutant KRAS, eighteen antigenic fragments of mutant KRAS, nineteen antigenic fragments of mutant KRAS, or twenty antigenic fragments of mutant KRAS, wherein each antigenic fragment comprises the mutation of one of the mutant KRAS proteins. In certain embodiments, each antigenic fragment comprises the same mutation or a different mutation of mutant KRAS proteins. In a specific embodiment, each fragment comprises a different mutation of KRAS. In a more specific embodiment, the arenavirus particle comprises an arenavirus genome comprising a nucleotide sequence encoding five antigenic fragments of mutant KRAS, wherein each antigenic fragment comprises a different mutation of mutant KRAS, wherein the different mutations are G13D, G12V, G12C, G12D, and G12R, wherein each antigenic fragment is 18 amino acids in length.

In certain embodiments, the antigenic fragments of mutant KRAS are directly fused together. In certain embodiments, the antigenic fragments of mutant KRAS are fused together via the same or different peptide linker. In specific embodiments, the antigenic fragments of mutant KRAS are fused together via AAY linker (AAY), AAA linker (AAA), GS linker (GGSGGGGSGG) (SEQ ID NO:42), or variants of AAY, AAA, and GS linker sequences optimized via in silico prediction.

In certain embodiments, the nucleotide sequence of the arenavirus particle is engineered to reduce or remove any CpG and TpA islands. In specific embodiments, the removal of the removal of the CpG and TpA islands comprises three cycles: (i) CpG is removed in a first cycle; (ii) TpA is removed in a second cycle; and (iii) CpG is removed in a third cycle to remove newly introduced CpG in the second cycle.

### 5.8 Mutated Cancer Driver Genes

In the context of this application “mutated cancer driver gene” means a polypeptide encoded by a mutated cancer driver gene.

In certain embodiments, the mutated cancer driver gene for use with the methods and compositions disclosed herein includes mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53, and mutant CTNNB1.

In certain embodiments, arenavirus particles with a nucleotide sequence encoding an antigenic fragment of a mutant KRAS that further comprise a nucleotide sequence encoding one or more antigenic fragment(s) of mutated cancer driver gene(s) (e.g., mutant TP53(s)) (i.e., the same arenavirus particle comprising different nucleotide sequences) are provided herein and can be used with the methods and compositions provided herein. In certain embodiments, arenavirus particles with a nucleotide sequence encoding an antigenic fragment of a mutant KRAS and one or more antigenic fragment(s) of mutated cancer driver gene(s) (e.g., mutant TP53(s)) (i.e., the same nucleotide sequence encoding different antigenic fragments) are provided herein and can be used with the methods and compositions provided herein. In certain embodiments, arenavirus particles with a nucleotide sequence encoding an antigenic fragment of mutant KRAS can further encode an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, wherein the antigenic fragment comprises the respective mutation. In certain embodiments, arenavirus particles with a nucleotide sequence encoding an antigenic fragment of mutant KRAS can further encode an antigenic fragment of mutant PI3KCA. In particular, the mutation in PI3KCA is E545K, H1047R, and/or E542K. In certain embodiments, arenavirus particles with a nucleotide sequence encoding an antigenic fragment of mutant KRAS can further encode an antigenic fragment of mutant BRAF. In particular, the mutation in BRAF is V600E. In certain embodiments, arenavirus particles with a nucleotide sequence encoding an antigenic fragment of mutant KRAS can further encode an antigenic fragment of mutant U2AF1. In particular, the mutation in U2AF1 is S34F. In certain embodiments, arenavirus particles with a nucleotide sequence encoding an antigenic fragment of mutant KRAS can further encode an antigenic fragment of mutant TP53. In particular, the mutation in TP53 is G245S, Y220C, R248Q, R282W, H179R, V157F, R273C, R213L, R273H, R273L, R175H, R158L, R196P, R248W, and/or C277F mutation. In certain embodiments, arenavirus particles with a nucleotide sequence encoding an antigenic fragment of mutant KRAS can further encode an antigenic fragment of mutant PI3KCA having a E545K mutation, H1047R mutation, or E542K mutation, mutant BRAF having a V600E mutation, or mutant TP53 having a R175H mutation.

In certain embodiments, the method for treating a neoplastic disease further comprises administering a second arenavirus particle, wherein the second arenavirus particle encodes an antigenic fragment of mutated cancer driver gene (e.g., a mutant TP53). In certain embodiments, an antigenic fragment of mutant KRAS, mutant BRAF having a mutation in V600E, or a mutant PIK3CA encoded by the genome of an arenavirus particle can be associated with or can be specific to colorectal cancer. In certain embodiments, an antigenic fragment of mutant KRAS, mutant BRAF, or mutant PIK3CA encoded by the genome of an arenavirus particle can be associated with or can be specific to lung adenocarcinoma. In certain embodiments, an antigenic fragment of mutant KRAS and mutant PIK3CA encoded by the genome of an arenavirus particle can be associated with or can be specific to lung squamous cell carcinoma. In certain embodiments, an antigenic fragment of mutant AKT1, mutant BRAF, mutant HER2, mutant MEK1, mutant MET, mutant NRAS, mutant PIK3CA, or mutant RET encoded by the genome of an arenavirus particle can be associated with or can be specific to non-small cell lung cancer (NSCLC).

In certain embodiments, the nucleotide sequence encodes a fragment of mutated cancer driver gene (e.g., a mutant TP53), wherein the fragment is 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, 26 amino acids in length, 27 amino acids in length, 28 amino acids in length, 29 amino acids in length, or 30 amino acids in length; and wherein the fragment comprises the mutation of the mutated cancer driver gene (e.g., a mutant TP53).

In certain embodiments, the nucleotide sequence encodes a fragment of mutated cancer driver gene (e.g., a mutant TP53), wherein the fragment is 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, 26 amino acids in length, 27 amino acids in length, 28 amino acids in length, 29 amino acids in length, or 30 amino acids in length; and wherein the fragment comprises the mutation of the mutated cancer driver gene (e.g., a mutant TP53).

In certain embodiments, the region flanking the mutation at the N-terminus of the antigenic fragment is 0 amino acids in length, 1 amino acid in length, 2 amino acids in length, 3 amino acids in length, 4 amino acids in length, 5 amino acids in length, 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, or 20 amino acids in length. In certain embodiments, the region flanking the mutation at the C-terminus of the antigenic fragment is 0 amino acids in length, 1 amino acid in length, 2 amino acids in length, 3 amino acids in length, 4 amino acids in length, 5 amino acids in length, 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, or 20 amino acids in length.

In certain embodiments, the arenavirus particles with a nucleotide sequence encoding two antigenic fragments of mutated cancer driver gene (e.g., a mutant TP53), three antigenic fragments of mutated cancer driver gene (e.g., a mutant TP53), four antigenic fragments of mutated cancer driver gene (e.g., a mutant TP53), five antigenic fragments of mutated cancer driver gene (e.g., a mutant TP53), six antigenic fragments of mutated cancer driver gene (e.g., a mutant TP53), seven antigenic fragments of mutated cancer driver gene (e.g., a mutant TP53), eight antigenic fragments of mutated cancer driver gene (e.g., a mutant TP53), nine antigenic fragments of mutated cancer driver gene (e.g., a mutant TP53), ten antigenic fragments of mutated cancer driver gene (e.g., a mutant TP53), eleven antigenic fragments of mutated cancer driver gene (e.g., a mutant TP53), twelve antigenic fragments of mutated cancer driver gene (e.g., a mutant TP53), thirteen antigenic fragments of mutated cancer driver gene (e.g., a mutant TP53), fourteen antigenic fragments of mutated cancer driver gene (e.g., a mutant TP53), fifteen antigenic fragments of mutated cancer driver gene (e.g., a mutant TP53), sixteen antigenic fragments of mutated cancer driver gene (e.g., a mutant TP53), seventeen antigenic fragments of mutated cancer driver gene (e.g., a mutant TP53), eighteen antigenic fragments of mutated cancer driver gene (e.g., a mutant TP53), nineteen antigenic fragments of mutated cancer driver gene (e.g., a mutant TP53), or twenty antigenic fragments of mutated cancer driver gene (e.g., a mutant TP53), wherein each antigenic fragment comprises the mutation of one of the mutated cancer driver gene (e.g., a mutant TP53) proteins. In certain embodiments, each antigenic fragment comprises the same mutation or a different mutation of the mutated cancer driver gene (e.g., a mutant TP53) proteins. In a specific embodiment, each fragment comprises a different mutation of cancer driver gene.

In certain embodiments, the antigenic fragments of mutated cancer driver gene (e.g., a mutant TP53) are directly fused together. In certain embodiments, the antigenic fragments of mutated cancer driver gene (e.g., a mutant TP53) are fused together via the same or different peptide linker. In specific embodiments, the antigenic fragments of mutated cancer driver gene (e.g., a mutant TP53) are fused together via AAY linker (AAY), AAA linker (AAA), GS linker (GGSGGGGSGG) (SEQ ID NO:42), or variants of AAY, AAA, and GS linker sequences optimized via in silico prediction.

In the context of this application “mutant TP53” means a polypeptide encoded by a mutated TP53 gene. In certain embodiments, arenavirus particles with a nucleotide sequence encoding an antigenic fragment of a mutant KRAS that further comprise a nucleotide sequence encoding one or more antigenic fragment(s) of one or more mutant TP53 (i.e., the same arenavirus particle comprising different nucleotide sequences) are provided herein and can be used with the methods and compositions provided herein. In certain embodiments, arenavirus particles with a nucleotide sequence encoding an antigenic fragment of a mutant KRAS and one or more antigenic fragment(s) of one or more mutant TP53 (i.e., the same nucleotide sequence encoding different antigenic fragments) are provided herein and can be used with the methods and compositions provided herein. In certain embodiments, arenavirus particles with a nucleotide sequence encoding an antigenic fragment of mutant KRAS can further encode an antigenic fragment of mutant TP53, wherein the antigenic fragment comprises the respective mutation. In certain embodiments, arenavirus particles with a nucleotide sequence encoding an antigenic fragment of mutant KRAS can further encode an antigenic fragment of mutant TP53. In particular, the mutation in TP53 is G245S, Y220C, R248Q, R282W, H179R, V157F, R273C, R213L, R273H, R273L, R175H, R158L, R196P, R248W, or C277F mutation.

In certain embodiments, the method for treating a neoplastic disease comprises administering an arenavirus particle, wherein the arenavirus particle encodes an antigenic fragment of mutant KRAS and mutant TP53, wherein the antigenic fragment comprises the mutation, wherein the mutation in KRAS is G12D, G12R, G12V, Q61H and/or Q61R, wherein the mutation in TP53 is R175H, R248W, G245S, R282W, R248Q and/or R273C, and wherein the neoplastic disease is pancreatic cancer. In certain embodiments, the method for treating a neoplastic disease comprises administering an arenavirus particle, wherein the arenavirus particle encodes an antigenic fragment of mutant KRAS and mutant TP53, wherein the antigenic fragment comprises the mutation, wherein the mutation in KRAS is G12D, G12V, G13D, A146T and/or G12C, wherein the mutation in TP53 is R175H, R273H and/or R248W, and wherein the neoplastic disease is colorectal cancer or lung adenocarcinoma. In certain embodiments, the method for treating a neoplastic disease comprises administering an arenavirus particle, wherein the arenavirus particle encodes an antigenic fragment of mutant KRAS and mutant TP53, wherein the antigenic fragment comprises the mutation, wherein the mutation in KRAS is G12C, G12D, G12R, G13D and/or G12V, wherein the mutation in TP53 is R175H, R273H and/or R248W, and wherein the neoplastic disease is pancreatic cancer, colorectal cancer or lung adenocarcinoma. In certain embodiments, the method for treating a neoplastic disease comprises administering an arenavirus particle, wherein the arenavirus particle encodes an antigenic fragment of mutant KRAS and mutant TP53, wherein the antigenic fragment comprises the mutation, wherein the mutation in KRAS is G12C, G12V, G12D, and/or G12S, wherein the mutation in TP53 is R175H, and wherein the neoplastic disease is lung adenocarcinoma. In certain embodiments, the method for treating a neoplastic disease further comprises administering a second arenavirus particle, wherein the second arenavirus particle encodes an antigenic fragment of mutant KRAS and mutant TP53, wherein the antigenic fragment comprises the mutation, wherein the mutation in KRAS is G12D, G12R, G12V, Q61H and/or Q61R, wherein the mutation in TP53 is R175H, R248W, G245S, R282W, R248Q and/or R273C, and wherein the neoplastic disease is pancreatic cancer. In certain embodiments, the method for treating a neoplastic disease further comprises administering a second arenavirus particle, wherein the second arenavirus particle encodes an antigenic fragment of mutant KRAS and mutant TP53, wherein the antigenic fragment comprises the mutation, wherein the mutation in KRAS is G12D, G12R, G12V, Q61H and/or Q61R, wherein the mutation in TP53 is R175H, R248W, G245S, R282W, R248Q and/or R273C, and wherein the neoplastic disease is pancreatic cancer. In certain embodiments, the method for treating a neoplastic disease further comprises administering a second arenavirus particle, wherein the second arenavirus particle encodes an antigenic fragment of mutant KRAS and mutant TP53, wherein the antigenic fragment comprises the mutation, wherein the mutation in KRAS is G12D, G12V, G13D, A146T and/or G12C, wherein the mutation in TP53 is R175H, R273H and/or R248W, and wherein the neoplastic disease is colorectal cancer or lung adenocarcinoma. In certain embodiments, the method for treating a neoplastic disease further comprises administering a second arenavirus particle, wherein the second arenavirus particle encodes an antigenic fragment of mutant KRAS and mutant TP53, wherein the antigenic fragment comprises the mutation, wherein the mutation in KRAS is G12C, G12D, G12R, G13D and/or G12V, wherein the mutation in TP53 is R175H, R273H and/or R248W, and wherein the neoplastic disease is pancreatic cancer, colorectal cancer or lung adenocarcinoma. In certain embodiments, the method for treating a neoplastic disease further comprises administering a second arenavirus particle, wherein the second arenavirus particle encodes an antigenic fragment of mutant KRAS and mutant TP53, wherein the antigenic fragment comprises the mutation, wherein the mutation in KRAS is G12C, G12V, G12D, and/or G12S, wherein the mutation in TP53 is R175H, and wherein the neoplastic disease is lung adenocarcinoma.

### 5.9 Tumor-Associated Antigens

In certain embodiments, the tumor-associated antigen for use with the methods and compositions disclosed herein includes antigens derived from the BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family, and XBP family.

In certain embodiments, arenavirus particles with a nucleotide sequence encoding an antigenic fragment of a mutant KRAS that further comprise a nucleotide sequence encoding one or more antigenic fragment(s) of tumor-associated antigen(s) (i.e., the same arenavirus particle comprising different nucleotide sequences) are provided herein and can be used with the methods and compositions provided herein. In certain embodiments, arenavirus particles with a nucleotide sequence encoding an antigenic fragment of a mutant KRAS and one or more antigenic fragment(s) of tumor-associated antigen(s) (i.e., the same nucleotide sequence encoding different antigenic fragments) are provided herein and can be used with the methods and compositions provided herein. In certain embodiments, a tumor-associated antigen for use with the methods and compositions described herein is an immunogenic protein expressed in or on a neoplastic cell or tumor, such as a cancer cell or malignant tumor. In certain embodiments, a tumor-associated antigen for use with the methods and compositions described herein is a non-specific, mutant, overexpressed or abnormally expressed protein, which can be present on both a neoplastic cell or tumor and a normal cell or tissue. In certain embodiments, a tumor-associated antigen for use with the methods and compositions described herein is a tumor-specific antigen which is restricted to tumor cells. In certain embodiments, a tumor-associated antigen for use with the methods and compositions described herein is a cancer-specific antigen which is restricted to cancer cells. In certain embodiments, the method for treating a neoplastic disease further comprises administering a second arenavirus particle, wherein the second arenavirus particle encodes an antigenic fragment of a tumor-associated antigen.

In certain embodiments, a tumor-associated antigen can exhibit one, two, three, or more, including all, of the following characteristics: overexpressed/accumulated (i.e., expressed by both normal and neoplastic tissue, but highly expressed in neoplasia), oncofetal (i.e., usually only expressed in fetal tissues and in cancerous somatic cells), oncoviral or oncogenic viral (i.e., encoded by tumorigenic transforming viruses), cancer-testis (i.e., expressed only by cancer cells and adult reproductive tissues, e.g., the testis), lineage-restricted (i.e., expressed largely by a single cancer histotype), mutated (i.e., only expressed in neoplastic tissue as a result of genetic mutation or alteration in transcription), post-translationally altered (e.g., tumor-associated alterations in glycosylation), or idiotypic (i.e., developed from malignant clonal expansions of B or T lymphocytes).

In certain embodiments, a tumor-associated antigen for use with the methods and compositions described herein includes antigens from neoplastic diseases including acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In certain embodiments, arenavirus particles with a nucleotide sequence encoding an antigenic fragment of mutant KRAS can further encode an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family, and XBP family.

In certain embodiments, the fragment of the tumor-associated antigen is antigenic when it is capable of (i) eliciting an antibody immune response in a host (e.g., mouse, rabbit, goat, donkey or human) wherein the resulting antibodies bind specifically to an immunogenic protein expressed in or on a neoplastic cell (e.g., a cancer cell); and/or (ii) eliciting a specific T cell immune response.

In certain embodiments, the nucleotide sequence encoding an antigenic fragment of a tumor-associated antigen provided herein is 8 to 100 nucleotides in length, 15 to 100 nucleotides in length, 25 to 100 nucleotides in length, 50 to 200 nucleotides in length, 50 to 400 nucleotides in length, 200 to 500 nucleotides in length, or 400 to 600 nucleotides in length, 500 to 800 nucleotides in length. In other embodiments, the nucleotide sequence encoding an antigenic fragment provided herein is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the nucleotide sequence encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the nucleotide sequence encodes a polypeptide that does not exceed 2500 amino acids in length. In specific embodiments the nucleotide sequence does not contain a stop codon. In certain embodiments, the nucleotide sequence is codon-optimized. In certain embodiments the nucleotide composition, nucleotide pair composition or both can be optimized. Techniques for such optimizations are known in the art and can be applied to optimize a nucleotide sequence encoding a tumor-associated antigen, or antigenic fragment thereof.

In certain embodiments, the arenavirus genomic segment, the arenavirus particle or the tri-segmented arenavirus particle can comprise one or more nucleotide sequences encoding tumor associated antigens, or antigenic fragments thereof. In other embodiments, the arenavirus genomic segment, the arenavirus particle or the tri-segmented arenavirus particle can comprise at least one nucleotide sequence encoding a tumor associated antigen, or antigenic fragment thereof, at least two nucleotide sequences encoding tumor associated antigens, or antigenic fragments thereof, at least three nucleotide sequences encoding tumor associated antigens, or antigenic fragments thereof, or more nucleotide sequences encoding tumor associated antigens, or antigenic fragments thereof.

In certain embodiments, an arenavirus particle provided herein comprises a genomic segment that a) has a removal or functional inactivation of an ORF that is present in the wild-type form of the genomic segment; and b) encodes (either in sense or antisense): (i) one or more mutant KRAS(s), mutated cancer driver gene(s), or tumor-associated antigen(s) provided herein. In other embodiments, an arenavirus particle provided herein comprises a genomic segment that a) comprises an arenaviral ORF in a position other than the wild-type position of the ORF; and b) encodes (either in sense or antisense): (i) one or more mutant KRAS(s), mutated cancer driver gene(s), or tumor-associated antigen(s) provided herein.

In certain embodiments, an arenavirus particle generated to encode one or more mutant KRASs, mutated cancer driver genes, or tumor-associated antigens comprises one or more nucleotide sequences encoding mutant KRASs, mutated cancer driver genes, or tumor-associated antigens provided herein. In specific embodiments, the mutant KRASs, mutated cancer driver genes, or tumor-associated antigens provided herein are separated by various one or more linkers, spacers, or cleavage sites as described herein.

### 5.10 Methods of Use

Provided herein are methods for preventing and/or treating a neoplastic disease in a subject comprising administering an arenavirus particle to a subject, wherein the arenavirus particle encodes a mutant KRAS (see section 5.7) provided herein. In certain embodiments, the arenavirus particle encodes a mutant KRAS, a mutated cancer driver gene (see section 5.8, e.g., a mutant TP53), or a tumor-associated antigen (see section 5.9) provided herein. In certain embodiments, these methods result in a reduction of tumor growth. In certain embodiments, these methods result in a lower rate of relapse.

In certain embodiments, provided herein are methods for treating a neoplastic disease in a subject comprising (a) administering a first arenavirus particle to a subject, wherein the first arenavirus particle encodes a mutant KRAS (see section 5.7), a mutated cancer driver gene (see section 5.8, e.g., a mutant TP53) and/or a tumor-associated antigen (see section 5.9); and (b) administering a second arenavirus particle to a subject, wherein the second arenavirus particle expresses a mutant KRAS, mutated cancer driver gene (e.g., a mutant TP53) and/or tumor-associated antigen. In certain embodiments, (a) and (b) is repeated for 1 time, 2 times, 3 times, 4 times, 5 times, 6 times, 7 times, 8 times, 9 times, or 10 times. In some embodiments, the first and the second arenavirus particle are administered simultaneously. In other embodiments, the interval between (a) and (b) is 1 week, 2 weeks, 3 weeks, 4 weeks, 5 weeks, 6 weeks, 7 weeks, 8 weeks, 9 weeks, 10 weeks, 11 weeks, or 12 weeks. In other embodiments, the interval between (a) and (b) is 13 weeks, 14 weeks, 15 weeks, 16 weeks, 17 weeks, 18 weeks, 19 weeks, 20 weeks, 22 weeks, 23 weeks, or 24 weeks. In other embodiments, the interval between (a) and (b) is 25 weeks, 26 weeks, 27 weeks, 28 weeks, 29 weeks, 30 weeks, 31 weeks, 32 weeks, 33 weeks, 34 weeks, 35 weeks, or 36 weeks. Furthermore, during the repeats of (a) and (b), the interval can be the same as the original cycle of (a) and (b), or can be different from the original cycle of (a) and (b). Accordingly, the interval between the (a) and (b) in the repeats can be 1 week, 2 weeks, 3 weeks, 4 weeks, 5 weeks, 6 weeks, 7 weeks, 8 weeks, 9 weeks, 10 weeks. In certain embodiments, the first arenavirus particle and the second arenavirus particle encode the same antigen(s), for example, two arenavirus particles encode the same mutant KRAS(s), mutated cancer driver gene(s) (e.g., mutant TP53(s)) and/or tumor-associated antigen(s). In certain embodiments, the first arenavirus particle and the second arenavirus particle encode different antigens, for example, a first arenavirus particle encodes a mutant KRAS only and a second arenavirus particle encodes a mutant KRAS, a mutated cancer driver gene (e.g., a mutant TP53) and/or a tumor-associated antigen provided herein. In certain embodiments, a first arenavirus particle encodes a mutant KRAS only and a second arenavirus particle encodes a mutated cancer driver gene (e.g., a mutant TP53) and/or a tumor-associated antigen. In certain embodiments, a first arenavirus particle encodes a mutant KRAS, a mutated cancer driver gene (e.g., a mutant TP53) and/or a tumor-associated antigen, and a second arenavirus particle encodes a mutant KRAS only. In certain embodiments, a first arenavirus particle encodes a mutant KRAS, a mutated cancer driver gene (e.g., a mutant TP53) and/or a tumor-associated antigen, and a second arenavirus particle encodes a mutated cancer driver gene (e.g., a mutant TP53) and/or a tumor-associated antigen.

In certain embodiments, the first arenavirus particle and the second arenavirus particle are the same. In certain embodiments, the first arenavirus particle and the second arenavirus particle are derived from the same arenavirus (that is, with the same backbone), but express different mutant KRASs (see section 5.7), mutated cancer driver genes (see section 5.8, e.g., a mutant TP53), or tumor-associated antigens (see section 5.9). In certain embodiments, the first arenavirus particle and the second arenavirus particle are derived from different arenaviruses (that is, with different backbones), but express the same mutant KRAS(s), mutant TP53(s), mutated cancer driver gene(s), or tumor-associated antigen(s). In certain embodiments, the first arenavirus particle and the second arenavirus particle are derived from different arenaviruses (that is, with different backbones), and express different mutant KRASs, mutant TP53(s), mutated cancer driver genes, or tumor-associated antigens.

In certain embodiment, provided herein are methods for treating a neoplastic disease in a subject comprising (a) administering a first arenavirus particle to the subject, wherein the first arenavirus particle is replication-competent and expresses a mutant KRAS (see section 5.7), a mutated cancer driver gene (see section 5.8, e.g., a mutant TP53), or a tumor-associated antigen (see section 5.9) provided herein; and (b) administering a second arenavirus particle to the subject, wherein the second arenavirus particle expresses a mutant KRAS, a mutated cancer driver gene (e.g., a mutant TP53), or a tumor-associated antigen. In certain embodiments, the first arenavirus particle is tri-segmented (see section 5.6(b)). In specific embodiments, the first arenavirus particle is replication-competent. In specific embodiments, the second arenavirus particle is replication-defective. In specific embodiments, the second arenavirus particle is replication-competent. In certain embodiments, the second arenavirus particle is tri-segmented. In specific embodiments, the second arenavirus particle is tri-segmented and replication-competent.

In certain embodiments, provided herein are methods of treating a neoplastic disease in a subject comprising administering an arenavirus particle comprising a nucleotide sequence encoding an antigenic fragment of mutant KRAS (see section 5.7), wherein the neoplastic disease is pancreatic cancer, colorectal cancer, lung adenocarcinoma, lung squamous cell carcinoma, or non-small cell lung cancer (NSCLC). In certain embodiments, the arenavirus particle comprises one or more nucleotide sequence(s) encoding an antigenic fragment of mutant KRAS (see section 5.8), wherein the antigenic fragment comprises the mutation, wherein the mutation in KRAS is G12D, G12V, G12R, Q61H, Q61R, G12C, G12S, and/or G12A, and wherein the neoplastic disease is pancreatic cancer. In certain embodiments, the arenavirus particle comprises one or more nucleotide sequence(s) encoding an antigenic fragment of mutant KRAS, mutant TP53 and/or mutant U2AF1 (see section 5.8), wherein the mutation in KRAS is G12D, G12V, G12R, Q61H and/or Q61R, wherein the mutation in TP53 is R175H, R248W, G245S, R282W, R248Q and/or R273C, wherein the mutation in U2AF1 is S34F, and wherein the neoplastic disease is pancreatic cancer. In certain embodiments, the arenavirus particle comprises one or more nucleotide sequence(s) encoding an antigenic fragment of mutant KRAS, wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma. In certain embodiments, the arenavirus particle comprises one or more nucleotide sequence(s) encoding an antigenic fragment of mutant KRAS, wherein the mutation in KRAS is G13D, G12V, G12C, G12D, and G12R and wherein the neoplastic disease is pancreatic cancer, colorectal cancer or lung adenocarcinoma. In certain embodiments, the arenavirus particle comprises one or more nucleotide sequence(s) encoding an antigenic fragment of mutant KRAS, mutant BRAF, mutant TP53, mutant FBXW7, and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is colorectal cancer or lung adenocarcinoma. In certain embodiments, the arenavirus particle comprises one or more nucleotide sequence(s) encoding an antigenic fragment of mutant KRAS, mutant BRAF, mutant PIK3CA, mutant EGFR, mutant TP53, and/or mutant U2AF1, wherein the antigenic fragment comprises the mutation, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, wherein the mutation in U2AF1 is S34F, and wherein the neoplastic disease is pancreatic cancer, colorectal cancer or lung adenocarcinoma. In certain embodiments, the arenavirus particle comprises one or more nucleotide sequence(s) encoding an antigenic fragment of mutant KRAS, mutant TP53, mutant U2AF1, mutant PIK3CA, mutant EGFR and/or mutant BRAF, wherein the antigenic fragment comprises the mutation, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E, and wherein the neoplastic disease is lung adenocarcinoma. In certain embodiments, the arenavirus particle comprises one or more nucleotide sequence(s) encoding an antigenic fragment of mutant AKT1, mutant BRAF, mutant HER2, mutant MEK1, mutant MET, mutant NRAS, mutant PIK3CA and/or mutant RET, wherein the antigenic fragment comprises the mutation, and wherein the neoplastic disease is non-small cell lung cancer (NSCLC). In certain embodiments, the arenavirus particle comprises one or more nucleotide sequence(s) encoding an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation. In certain embodiments, the arenavirus particle comprises one or more nucleotide sequence(s) encoding an antigenic fragment of mutant U2AF1, wherein the antigenic fragment comprises the mutation, and wherein the mutation in U2AF1 is S34F. In certain embodiments, the arenavirus particle comprises one or more nucleotide sequence(s) encoding an antigenic fragment of mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W or C277F. In certain embodiments, the arenavirus particle comprises one or more nucleotide sequence(s) encoding an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family (see section 5.9), and wherein the antigenic fragment comprises the mutation.

In certain embodiments, provided herein are methods of treating a neoplastic disease in a subject comprising further administering a second arenavirus particle, wherein the second arenavirus particle comprises a nucleotide sequence encoding antigenic fragments of mutant KRAS, wherein the antigenic fragments comprise the mutation, wherein the mutation in KRAS is G12D, G12V, G12R, Q61H, Q61R, G12C, G12S, and/or G12A and wherein the neoplastic disease is pancreatic cancer. In certain embodiments, the second arenavirus particle comprises one or more nucleotide sequence(s) encoding an antigenic fragment of mutant KRAS, mutant TP53, and/or mutant U2AF1, wherein the antigenic fragment comprises the mutation, wherein the mutation in KRAS is G12D, G12V, G12R, Q61H and/or Q61R, wherein the mutation in TP53 is R175H, R248W, G245S, R282W, R248Q and/or R273C, wherein the mutation in U2AF1 is S34F, and wherein the neoplastic disease is pancreatic cancer. In certain embodiments, the second arenavirus particle comprises one or more nucleotide sequence(s) encoding antigenic fragments of mutant KRAS, wherein the antigenic fragment comprises the mutation, and wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma. In certain embodiments, the second arenavirus particle comprises one or more nucleotide sequence(s) encoding antigenic fragments of mutant KRAS, wherein the antigenic fragment comprises the mutation, and wherein the mutation in KRAS is G13D, G12V, G12C, G12D, and G12R and wherein the neoplastic disease is pancreatic cancer, colorectal cancer or lung adenocarcinoma. In certain embodiments, the second arenavirus particle comprises one or more nucleotide sequence(s) encoding an antigenic fragment of mutant KRAS, mutant BRAF, mutant TP53, mutant FBXW7 and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is colorectal cancer or lung adenocarcinoma. In certain embodiments, the second arenavirus particle comprises one or more nucleotide sequence(s) encoding an antigenic fragment of mutant KRAS, mutant BRAF, mutant PIK3CA, mutant EGFR, mutant TP53, and/or mutant U2AF1, wherein the antigenic fragment comprises the mutation, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, wherein the mutation in U2AF1 is S34F, and wherein the neoplastic disease is pancreatic cancer, colorectal cancer or lung adenocarcinoma. In certain embodiments, the second arenavirus particle comprises one or more nucleotide sequence(s) encoding an antigenic fragment of mutant KRAS, mutant TP53, mutant U2AF1, mutant PIK3CA, mutant EGFR and/or mutant BRAF, wherein the antigenic fragment comprises the mutation, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E, and wherein the neoplastic disease is lung adenocarcinoma. In certain embodiments, the second arenavirus particle comprises one or more nucleotide sequence(s) encoding an antigenic fragment of mutant AKT1, mutant BRAF, mutant HER2, mutant MEK1, mutant MET, mutant NRAS, mutant PIK3CA and/or mutant RET, wherein the antigenic fragment comprises the mutation, and wherein the neoplastic disease is non-small cell lung cancer (NSCLC). In certain embodiments, the second arenavirus particle comprises one or more nucleotide sequence(s) encoding an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant FBXW7, mutant TP53 and/or mutant CTNNB1 (see section 5.8), wherein the antigenic fragment comprises the mutation. In certain embodiments, the second arenavirus particle comprises a nucleotide sequence encoding an antigenic fragment of mutant U2AF1, wherein the antigenic fragment comprises the mutation, and wherein the mutation in U2AF1 is S34F. In certain embodiments, the second arenavirus particle comprises a nucleotide sequence encoding an antigenic fragment of mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W and/or C277F (see section 5.8). In certain embodiments, the second arenavirus particle comprises one or more nucleotide sequence(s) encoding an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family (see section 5.9).

In certain embodiments, provided herein are methods of treating a neoplastic disease in a subject comprising administering an arenavirus particle comprising a nucleotide sequence encoding an antigenic fragment of mutant KRAS (see section 5.7), wherein the neoplastic disease is pancreatic cancer, colorectal cancer, lung adenocarcinoma, lung squamous cell carcinoma, or non-small cell lung cancer (NSCLC). In certain embodiments, the arenavirus particle comprises one or more nucleotide sequence(s) encoding antigenic fragments of mutant KRAS, wherein the antigenic fragments comprise the mutation, and wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma. In certain embodiments, the arenavirus particle comprises one or more nucleotide sequence(s) encoding an antigenic fragment of mutant KRAS, wherein the mutation in KRAS is G13D, G12V, G12C, G12D, and G12R and wherein the neoplastic disease is pancreatic cancer, colorectal cancer or lung adenocarcinoma. In certain embodiments, the arenavirus particle comprises one or more nucleotide sequence(s) encoding antigenic fragments of mutant KRAS, wherein the antigenic fragments comprise the mutation, wherein the mutation in KRAS is G12D, G12V, G12R, Q61H, Q61R, G12C, G12S, and/or G12A and wherein the neoplastic disease is pancreatic cancer. In certain embodiments, the arenavirus particle comprises one or more nucleotide sequence(s) encoding an antigenic fragment of mutant KRAS and/or mutant TP53, wherein the antigenic fragment comprises the mutation, wherein the mutation in KRAS is G12D, G12R. G12V, Q61H and/or Q61R, wherein the mutation in TP53 is R175H, R248W, G245S, R282W, R248Q and/or R273C, and wherein the neoplastic disease is pancreatic cancer. In certain embodiments, the arenavirus particle comprises one or more nucleotide sequence(s) encoding an antigenic fragment of mutant KRAS and/or mutant TP53, wherein the antigenic fragment comprises the mutation, wherein the mutation in KRAS is G12D, G12V, G13D, A146T and/or G12C, wherein the mutation in TP53 is R175H, R273H and/or R248W, and wherein the neoplastic disease is colorectal cancer or lung adenocarcinoma. In certain embodiments, the arenavirus particle comprises one or more nucleotide sequence(s) encoding an antigenic fragment of mutant KRAS and/or mutant TP53, wherein the antigenic fragment comprises the mutation, wherein the mutation in KRAS is G12C, G12D, G12R, G13D and/or G12V, wherein the mutation in TP53 is R175H, R273H and/or R248W, and wherein the neoplastic disease is pancreatic cancer, colorectal cancer or lung adenocarcinoma. In certain embodiments, the arenavirus particle comprises one or more nucleotide sequence(s) encoding an antigenic fragment of mutant KRAS and/or mutant TP53, wherein the antigenic fragment comprises the mutation, wherein the mutation in KRAS is G12C, G12V, G12D, and/or G12S, wherein the mutation in TP53 is R175H, and wherein the neoplastic disease is lung adenocarcinoma.

In certain embodiments, the arenavirus particle comprises one or more nucleotide sequence(s) encoding an antigenic fragment of mutant KRAS and/or mutant TP53, and wherein the antigenic fragment comprises the respective mutation. In certain embodiments, the arenavirus particle comprises one or more nucleotide sequence(s) encoding an antigenic fragment of mutant KRAS and/or mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W or C277F.

In certain embodiments, provided herein are methods of treating a neoplastic disease in a subject comprising further administering a second arenavirus particle, wherein the second arenavirus particle comprises a nucleotide sequence encoding an antigenic fragment of mutant KRAS (see section 5.7), wherein the antigenic fragment comprises the mutation, and wherein the neoplastic disease is pancreatic cancer. In certain embodiments, the second arenavirus particle comprises one or more nucleotide sequence(s) encoding an antigenic fragment of mutant KRAS, wherein the antigenic fragment comprises the mutation, wherein the mutation in KRAS is G12D, G12V, G12C, G12R, G13D, A146T, G12S, Q61H, G12A, and/or Q61R, and wherein the neoplastic disease is pancreatic cancer, colorectal cancer or lung adenocarcinoma.

In certain embodiments, the second arenavirus particle comprises one or more nucleotide sequence(s) encoding an antigenic fragment of mutant KRAS, wherein the antigenic fragment comprises the mutation, wherein the mutation in KRAS is G12D, G12V, G12R, Q61H, Q61R, G12C, G12S, and/or G12A, and wherein the neoplastic disease is pancreatic cancer.

In certain embodiments, the second arenavirus particle comprises one or more nucleotide sequence(s) encoding an antigenic fragment of mutant KRAS and/or mutant TP53, wherein the antigenic fragment comprises the mutation, wherein the mutation in KRAS is G12D, G12R, G12V, Q61H and/or Q61R, wherein the mutation in TP53 is R175H, R248W, G245S, R282W, R248Q and/or R273C, and wherein the neoplastic disease is pancreatic cancer. In certain embodiments, the second arenavirus particle comprises one or more nucleotide sequence(s) encoding an antigenic fragment of mutant KRAS and/or mutant TP53, wherein the antigenic fragment comprises the mutation, wherein the mutation in KRAS is G12D, G12V, G13D, A146T and/or G12C, wherein the mutation in TP53 is R175H, R273H and/or R248W, and wherein the neoplastic disease is colorectal cancer or lung adenocarcinoma. In certain embodiments, the second arenavirus particle comprises one or more nucleotide sequence(s) encoding an antigenic fragment of mutant KRAS and/or mutant TP53, wherein the antigenic fragment comprises the mutation, wherein the mutation in KRAS is G12C, G12D, G12R, G13D and/or G12V, wherein the mutation in TP53 is R175H, R273H and/or R248W, and wherein the neoplastic disease is pancreatic cancer, colorectal cancer or lung adenocarcinoma. In certain embodiments, the second arenavirus particle comprises one or more nucleotide sequence(s) encoding an antigenic fragment of mutant KRAS and/or mutant TP53, wherein the antigenic fragment comprises the mutation, wherein the mutation in KRAS is G12C, G12V, G12D, and/or G12S, wherein the mutation in TP53 is R175H, and wherein the neoplastic disease is lung adenocarcinoma. In certain embodiments, the second arenavirus particle comprises one or more nucleotide sequence(s) encoding an antigenic fragment of mutant KRAS and/or mutant TP53, wherein the antigenic fragment comprises the mutation. In certain embodiments, the second arenavirus particle comprises a nucleotide sequence encoding an antigenic fragment of mutant KRAS and/or mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W and/or C277F (see section 5.8).

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., TO, T is, or T1-4), state of metastasis (e.g., M0, M1), number of observable tumors, node involvement (e.g., NO, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, S-100, TA-90, and thyroglobulin), and/or associated pathologies (e.g., ascites or edema) or symptoms (e.g., cachexia, fever, anorexia, or pain). The improvement, if measurable by percent, can be at least 5, 10, 15, 20, 25, 30, 40, 50, 60, 70, 80, or 90% (e.g., survival, or volume or linear dimensions of a tumor).

In another embodiment, an arenavirus particle expressing a mutant KRAS (see section 5.7), a mutated cancer driver gene (see section 5.8, e.g., a mutant TP53), or a tumor-associated antigen (see section 5.9) provided herein is administered to a subject by intratumoral injection. In another embodiment, an arenavirus particle expressing a mutant KRAS, a mutated cancer driver gene (e.g., a mutant TP53), or a tumor-associated antigen provided herein is administered to a subject by intravenous injection.

In another embodiment, an arenavirus particle expressing a mutant KRAS (see section 5.7), a mutated cancer driver gene (see section 5.8, e.g., a mutant TP53), or a tumor-associated antigen (see section 5.9) provided herein, or a composition thereof, is administered to a subject with risk factors. Exemplary risk factors include aging, tobacco, sun exposure, radiation exposure, chemical exposure, family history, alcohol, poor diet, lack of physical activity, or being overweight.

In another embodiment, an arenavirus particle expressing a mutant KRAS (see section 5.7), a mutated cancer driver gene (see section 5.8, e.g., a mutant TP53), or a tumor-associated antigen (see section 5.9) provided herein, or a composition thereof, is administered to subjects who suffer from one or more types of cancers. In other embodiments, any type of neoplastic disease, such as cancer, that is susceptible to treatment with the compositions described herein might be targeted.

In another embodiment, administering an arenavirus particle expressing a mutant KRAS (see section 5.7), a mutated cancer driver gene (see section 5.8, e.g., a mutant TP53), or a tumor-associated antigen (see section 5.9) provided herein, or a composition thereof, to subjects confer cell-mediated immunity (CMI) against a neoplastic cell or tumor, such as a cancer cell or tumor. Without being bound by theory, in another embodiment, an arenavirus particle expressing a mutant KRAS, a mutated cancer driver gene (e.g., a mutant TP53), or a tumor-associated antigen provided herein, or a composition thereof, infects and expresses antigens of interest in antigen presenting cells (APC) of the host (e.g., macrophages) for direct presentation of antigens on Major Histocompatibility Complex (MHC) class I and II. In another embodiment, administering an arenavirus particle expressing a mutant KRAS, a mutated cancer driver gene (e.g., a mutant TP53), or a tumor-associated antigen provided herein, or a composition thereof, to subjects induces plurifunctional IFN-7 and TNF-α co-producing cancer-specific CD4+ and CD8+ T cell responses (IFN-7 is produced by CD4+ and CD8+ T cells and TNF-α is produced by CD4+ T cells) of high magnitude to treat a neoplastic disease.

In another embodiment, administering an arenavirus particle expressing a mutant KRAS (see section 5.7), a mutated cancer driver gene (see section 5.8, e.g., a mutant TP53), or a tumor-associated antigen (see section 5.9) provided herein, or a composition thereof, increases or improves one or more clinical outcomes for cancer treatment. Non-limiting examples of such outcomes are overall survival, progression-free survival, time to progression, time to treatment failure, event-free survival, time to next treatment, overall response rate and duration of response. The increase or improvement in one or more of the clinical outcomes can be by at least about 10%, at least about 20%, at least about 25%, at least about 30%, at least about 35%, at least about 40%, at least about 50%, at least about 60%, at least about 70%, at least about 80%, at least about 90%, or more, compared to a patient or group of patients having the same neoplastic disease in the absence of such treatment.

Changes in cell-mediated immunity (CMI) response function against a neoplastic cell or tumor, including a cancer cell or tumor, induced by administering an arenavirus particle expressing a mutant KRAS (see section 5.7), a mutated cancer driver gene (see section 5.8, e.g., a mutant TP53), or a tumor-associated antigen (see section 5.9) provided herein, or a composition thereof, in subjects can be measured by any assay known to the skilled artisan including, but not limited to flow cytometry (see, e.g., Perfetto S. P. et al., Nat Rev Immun. 2004; 4(8):648-55), lymphocyte proliferation assays (see, e.g., Bonilla F. A. et al., Ann Allergy Asthma Immunol. 2008; 101:101-4; and Hicks M. J. et al., Am J Clin Pathol. 1983; 80:159-63), assays to measure lymphocyte activation including determining changes in surface marker expression following activation of measurement of cytokines of T lymphocytes (see, e.g., Caruso A. et al., Cytometry. 1997; 27:71-6), ELISPOT assays (see, e.g., Czerkinsky C.C. et al., J Immunol Methods. 1983; 65:109-121; and Hutchings P.R., et al., J Immunol Methods. 1989; 120:1-8), or Natural killer cell cytotoxicity assays (see, e.g., Bonilla F. A. et al., Ann Allergy Asthma Immunol. 2005 May; 94(5 Suppl 1):S1-63).

In certain embodiments, the treatments provided herein can further be combined with a chemotherapeutic agent. Chemotherapeutic agents include alkylating agents (e.g., cyclophosphamide), platinum-based therapeutics, antimetabolites, topoisomerase inhibitors, cytotoxic antibiotics, intercalating agents, mitosis inhibitors, taxanes, or combinations of two or more thereof. In certain embodiments, the alkylating agent is a nitrogen mustard, a nitrosourea, an alkyl sulfonate, a non-classical alkylating agent, or a triazene. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above.

In certain embodiments, the one or more arenavirus particles expressing a mutant KRAS (see section 5.7), a mutated cancer driver gene (see section 5.8, e.g., a mutant TP53), or a tumor-associated antigen (see section 5.9) provided herein, or a composition thereof, are administered, in two or more separate injections over a 1-hour period, 2-hour period, 3-hour period, 6-hour period, a 12-hour period, a 24-hour period, or a 48-hour period.

In certain embodiments, the one or more arenavirus particles expressing a mutant KRAS (see section 5.7), a mutated cancer driver gene (see section 5.8, e.g., a mutant TP53), or a tumor-associated antigen (see section 5.9) provided herein, or a composition thereof, are administered, in two or more separate injections over a 3-day period, a 5-day period, a 1-week period, a 2-week period, a 3-week period, a 4-week period, or a 12-week period.

In certain embodiments, the one or more arenavirus particles expressing a mutant KRAS (see section 5.7), a mutated cancer driver gene (see section 5.8, e.g., a mutant TP53), or a tumor-associated antigen (see section 5.9) provided herein, or a composition thereof, are administered, in two or more separate injections over a 6-month period, a 12-month period, a 24-month period, or a 48-month period.

In certain embodiments, two arenavirus particles provided herein are administered in a treatment regime, administration may be at molar ratios ranging from about 1:1 to 1:1000, in particular including: 1:1 ratio, 1:2 ratio, 1:5 ratio, 1:10 ratio, 1:20 ratio, 1:50 ratio, 1:100 ratio, 1:200 ratio, 1:300 ratio, 1:400 ratio, 1:500 ratio, 1:600 ratio, 1:700 ratio, 1:800 ratio, 1:900 ratio, 1:1000 ratio.

In certain embodiments, provided herein is a method of treating a neoplastic disease wherein a first arenavirus particle is administered first as a “prime,” and a second arenavirus particle is administered as a “boost.” The first and the second arenavirus particles can express the same or different mutant KRASs, mutated cancer driver genes (e.g., mutant TP53s), or tumor-associated antigens. Alternatively, or additionally, in some certain embodiments, the “prime” and “boost” administration are performed with an arenavirus particle derived from different arenavirus species. In certain specific embodiments, the “prime” administration is performed with an arenavirus particle derived from LCMV, and the “boost” is performed with an arenavirus particle derived from Pichinde virus. In certain specific embodiments, the “prime” administration is performed with an arenavirus particle derived from Pichinde virus, and the “boost” is performed with an arenavirus particle derived from LCMV.

In certain embodiments, administering a first arenavirus particle expressing a mutant KRAS (see section 5.7), a mutated cancer driver gene (see section 5.8, e.g., a mutant TP53) and/or a tumor-associated antigen (see section 5.9), followed by administering a second arenavirus particle expressing a mutant KRAS, mutated cancer driver gene (e.g., a mutant TP53) and/or tumor-associated antigen results in a greater antigen specific CD8+ T cell response than administering a single arenavirus particle expressing a mutant KRAS, mutated cancer driver gene (e.g., a mutant TP53) and/or tumor-associated antigen. In certain embodiments, the antigen specific CD8+ T cell count increases by 50%, 100%, 150% or 200% after the second administration compared to the first administration.

In certain embodiments, administering a first arenavirus particle expressing a mutant KRAS (see section 5.7), a mutated cancer driver gene (see section 5.8, e.g., a mutant TP53) and/or a tumor-associated antigen (see section 5.9) and a second, heterologous, arenavirus particle expressing a mutant KRAS, mutated cancer driver gene (e.g., a mutant TP53) and/or tumor-associated antigen elicits a greater CD8+ T cell response than administering a first arenavirus particle expressing a mutant KRAS, mutated cancer driver gene (e.g., a mutant TP53) and/or tumor-associated antigen and a second, homologous, arenavirus particle expressing a mutant KRAS, mutated cancer driver gene (e.g., a mutant TP53) and/or tumor-associated antigen.

### 5.11 Compositions, Administration, and Dosage

Also provided herein are vaccines, immunogenic compositions (e.g., vaccine formulations), and pharmaceutical compositions comprising an arenavirus particle provided herein. Such vaccines, immunogenic compositions and pharmaceutical compositions can be formulated according to standard procedures in the art.

In certain embodiments, provided herein are immunogenic compositions comprising an arenavirus particle (or a combination of different arenavirus particles) as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an infectious, replication-deficient arenavirus particle, but when the compound is administered alone does not generate an immune response to the infectious, replication-deficient arenavirus particle. In some embodiments, the adjuvant generates an immune response to the infectious, replication-deficient arenavirus particle and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the genetically engineered arenavirus particles. Unit dose forms for parenteral administration are, for example, ampoules or vials, e.g., vials containing from about 103 to 10¹⁰ focus forming units or 10⁵ to 10¹⁵ physical particles of genetically engineered arenavirus particles.

In another embodiment, a vaccine or immunogenic composition provided herein is administered to a subject by intratumoral injection. In another embodiment, a vaccine or immunogenic composition provided herein is administered to a subject by intravenous injection.

The dosage of the active ingredient depends upon the type of vaccination and upon the subject, and their age, weight, individual condition, the individual pharmacokinetic data, and the mode of administration.

In certain embodiments, the composition is administered to the patient as a single dose followed by a second dose three to six weeks later. In accordance with these embodiments, the booster inoculations may be administered to the subjects at six to twelve months intervals following the second inoculation. In certain embodiments, the booster inoculations may utilize a different arenavirus particle or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

In certain embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and can be administered to a subject once, twice, three or more times with intervals as often as needed.

In certain embodiments, the methods and compositions provided herein are used in combination with personalized medicine. Personalized medicine seeks to benefit patients by using information from a patient's unique genetic and/or epigenetic profile to predict a patient's response to different therapies and identify which therapies are more likely to be effective. Techniques that can be used in combination with the methods and compositions provided herein to obtain a patient's unique genetic and/or epigenetic profile include, but are not limited to, genome sequencing, RNA sequencing, gene expression analysis and identification of a tumor antigen (e.g., neoantigen), tumor associated antigen or an antigenic fragment thereof. In certain embodiments, the selection of an arenavirus tumor antigen or tumor associated antigen for use in the methods and compositions provided herein is performed based on the genetic profile of the patient. In certain embodiments, the selection of an arenavirus tumor antigen or tumor associated antigen for use in the methods and compositions provided herein is performed based on the genetic profile of a tumor or tumor cell.

Also provided herein are kits that can be used to perform the methods described herein. In certain embodiments, the kit provided herein can include one or more containers. These containers can hold for storage the compositions (e.g., pharmaceutical, immunogenic or vaccine composition) provided herein. Also included in the kit are instructions for use. These instructions describe, in sufficient detail, a treatment protocol for using the compositions contained therein. For example, the instructions can include closing and administration instructions as provided herein for the methods of treating a neoplastic disease.

### 5.12 Assays to Demonstrate Activity

### (a) Arenavirus Detection Assays

The skilled artesian could detect an arenavirus genomic segment or an arenavirus particle, as described herein using techniques known in the art. For example, RT-PCR can be used with primers that are specific to an arenavirus to detect and quantify an arenavirus genomic segment or a tri-segmented arenavirus particle. Western blot, ELISA, radioimmunoassay, immunoprecipitation, immunocytochemistry, or immunocytochemistry in conjunction with FACS can be used to quantify the gene products of the arenavirus genomic segment or arenavirus particle.

### (b) Assay to Measure Infectivity

Any assay known to the skilled artisan can be used for measuring the infectivity of an arenavirus vector preparation. For example, determination of the virus/vector titer can be done by a “focus forming unit assay” (FFU assay). In brief, complementing cells, e.g., HEK293-TVL cells are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and thus countable using antibodies against LCMV-NP or another protein expressed by the arenavirus particle or the tri-segmented arenavirus particle and a HRP-based color reaction. The titer of a virus/vector can be calculated in focus-forming units per milliliter (FFU/mL). In a similar way, the proportion of tri-segmented, replication competent virus particles can be determined. Instead of complementing cells, non-complementing cell lines are used, e.g., HEK293. This allows only tri-segmented virus particles to infect neighboring cells. The titer of the replication competent virus/vector (RCV) can be calculated in focus-forming units per milliliter (RCV FFU/mL).

### (c) Growth of an Arenavirus Particle

Growth of an arenavirus particle described herein can be assessed by any method known in the art or described herein (e.g., cell culture). Viral growth may be determined by inoculating a defined amount/concentration of arenavirus particles described herein into cell cultures (e.g., Vero cells or BHK-21 cells). After incubation of the virus for a specified time, the virus containing supernatant is collected using standard methods and the infectivity can be measured using herein described assays.

### (d) Serum ELISA

Determination of the humoral immune response upon vaccination of animals (e.g., mice, guinea pigs) can be done by antigen-specific serum ELISAs (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

### (e) Assay to Measure the Neutralizing Activity of Induced Antibodies

Determination of the neutralizing antibodies in sera is performed with the following cell assay using ARPE-19 cells from ATCC and a GFP-tagged virus. In addition supplemental guinea pig serum as a source of exogenous complement is used. The assay is started with seeding of 6.5×10³ cells/well (50p1/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope.

### (f) Plaque Reduction Assay

In brief, plaque reduction (neutralization) assays for LCMV can be performed by use of a replication-competent or -deficient LCMV that is encoding a reporter gene (e.g., green fluorescent protein), 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples. qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be. 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### (g) Western Blotting

Infected cells grown in tissue culture flasks or in suspension are lysed at indicated time points post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogen's iBlot Gel transfer Device and visualized by Ponceau staining. Finally, the preparations are probed with a primary antibody directed against proteins of interest and alkaline phosphatase conjugated secondary antibodies followed by staining with 1-Step NBT/BCIP solution (INVITROGEN).

### (h) MHC-Peptide Multimer Staining Assay for Detection of Antigen-Specific CD8+ T-cells

Any assay known to the skilled artisan can be used to test antigen-specific CD8+ T-cell responses. For example, the MHC-peptide tetramer staining assay can be used (see, e.g., Altman J.D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order to detect an antigen-specific T-cell, it must bind to both, the peptide and the tetramer of MHC molecules custom made for a defined antigen specificity and MHC haplotype of T-cells (typically fluorescently labeled). The tetramer is then detected by flow cytometry via the fluorescent label.

### (i) ELISPOT Assay for Detection of Antigen-Specific T-cells

Any assay known to the skilled artisan can be used to test antigen-specific T-cell responses. For example, the ELISPOT assay can be used (see, e.g., Czerkinsky C.C. et al., J Immunol Methods. 1983; 65:109-121; and Hutchings P.R. et al., J Immunol Methods. 1989; 120:1-8). e.g., cytokines such as but not limited to IFN-7 can be measured by the ELISPOT assay. Briefly, the assay comprises the following steps: An immunospot plate is coated with an anti-cytokine antibody. Cells are incubated in the immunospot plate with peptides derived from the antigen of interest. Antigen-specific cells secrete cytokines which bind to the coated antibodies. The cells are then washed off and a second biotyinlated-anticytokine antibody is added to the plate and visualized with an avidin-HRP system or other appropriate methods.

### (j) Intracellular Cytokine Assay for Detection of Functionality of CD8+ and CD4+ T-cells

Any assay known to the skilled artisan can be used to test the functionality of CD8+ and CD4+ T cell responses. For example, the intracellular cytokine assay combined with flow cytometry can be used (see, e.g., Suni M.A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: upon activation of cells via specific peptides or protein, an inhibitor of protein transport (e.g., brefeldin A) is added to retain the cytokines within the cell. After a defined period of incubation, typically 5 hours, a washing step follows, and antibodies to other cellular markers can be added to the cells. Cells are then fixed and permeabilized. The fluorochrome-conjugated anti-cytokine antibodies are added and the cells can be analyzed by flow cytometry.

### (k) Assay for Confirming Replication-Deficiency of Viral Vectors

Any assay known to the skilled artisan that determines concentration of infectious and replication-competent virus particles can also be used to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32:Immunology of Infection. Academic Press. ISBN 0-12-521532-0). Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

### (1) Assay for Expression of Viral Antigen

Any assay known to the skilled artisan can be used for measuring expression of viral antigens. For example, FFU assays can be performed. For detection, mono- or polyclonal antibody preparation(s) against the respective viral antigens are used (transgene-specific FFU).

### (m) Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice can be used to investigate recombination and infectivity of an arenavirus particle are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal model. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene stability.

### 6. Equivalents

All patents and publications mentioned in this specification are incorporated herein by reference in their entireties. From the foregoing description, it will be apparent that variations and modifications can be made to the invention described herein to adopt it to various uses and conditions. Such embodiments are also within the scope of the following claims.

### 7. Sequences

| SEQ | | |
|---|---|---|
| ID | | |
| NO. | Description | Sequence |
| 1 | Nucleotide | ATGACAGAGTACAAACTGGTGGTGGTGGGT |
| | sequence of | GCTGTGGGGGTGGGAAAAAGTGCCTTGACC |
| | 4xKRASmut | ATTCAGCTCATTCAGAATCACTTTGTGGAC |
| | | ACAGAGTACAAGTTGGTGGTTGTCGGAGCA |
| | | GGTGATGTTGGAAAATCAGCCCTCACCATC |
| | | CAGCTGATCCAGAACCACTTTGTTGACACA |
| | | GAATACAAGCTGGTGGTTGTGGGGGCTGAT |
| | | GGAGTGGGCAAGAGTGCACTCACCATCCAA |
| | | CTGATCCAGAATCATTTTGTGGACACTGAG |
| | | TACAAGCTGGTTGTGGTGGGGGCCTGTGGG |
| | | GTGGGCAAATCAGCCCTCACCATTCAACTC |
| | | ATTCAAAACCATTTTGTGGATTGA |
| 2 | Nucleotide | ATGACAGAGTACAAACTGGTGGTGGTGGGT |
| | sequence of | GCTGTGGGGGTGGGAAAAAGTGCCTTGACC |
| | 4xKRASmut_E7 | ATTCAGCTCATTCAGAATCACTTTGTGGAC |
| | | ACAGAGTACAAGTTGGTGGTTGTCGGAGCA |
| | | GGTGATGTTGGAAAATCAGCCCTCACCATC |
| | | CAGCTGATCCAGAACCACTTTGTTGACACA |
| | | GAATACAAGCTGGTGGTTGTGGGGGCTGAT |
| | | GGAGTGGGCAAGAGTGCACTCACCATCCAA |
| | | CTGATCCAGAATCATTTTGTGGACACTGAG |
| | | TACAAGCTGGTTGTGGTGGGGGCCTGTGGG |
| | | GTGGGCAAATCAGCCCTCACCATTCAACTC |
| | | ATTCAAAACCATTTTGTGGATATGCATGGG |
| | | GACACCCCCACCCTGCATGAGTACATGCTG |
| | | GACCTCCAGCCTGAGACCACAGACCTGTAC |
| | | TGCTATGAGCAGCTGAACTGA |
| 3 | Nucleotide | ATGACAGAGTACAAACTGGTGGTGGTGGGT |
| | sequence of | GCTGTGGGGGTGGGAAAAAGTGCCTTGACC |
| | 4xKRASmut_ | ATTCAGCTCATTCAGAATCACTTTGTGGAC |
| | EBV | ACAGAGTACAAGTTGGTGGTTGTCGGAGCA |
| | | GGTGATGTTGGAAAATCAGCCCTCACCATC |
| | | CAGCTGATCCAGAACCACTTTGTTGACACA |
| | | GAATACAAGCTGGTGGTTGTGGGGGCTGAT |
| | | GGAGTGGGCAAGAGTGCACTCACCATCCAA |
| | | CTGATCCAGAATCATTTTGTGGACACTGAG |
| | | TACAAGCTGGTTGTGGTGGGGGCCTGTGGG |
| | | GTGGGCAAATCAGCCCTCACCATTCAACTC |
| | | ATTCAAAACCATTTTGTGGATGATGCCAAG |
| | | AGCACCAAGAAGTGCAGAGCCATTGTGACT |
| | | GACTTCAGTGTGATCAAGGCCATAGAGGAG |
| | | GAGCACAGAAAGAAGAAGTGA |
| 4 | Amino acid | MTEYKLVVVGAVGVGKSALTIQLIQNHFVD |
| | suence of | TEYKLVVVGAGDVGKSALTIQLIQNHFVDT |
| | 4xKRASmut | EYKLVVVGADGVGKSALTIQLIQNHFVDTE |
| | | YKLVVVGACGVGKSALTIQLIQNHFVD |
| 5 | Amino acid | MTEYKLVVVGAVGVGKSALTIQLIQNHFVD |
| | suence of | TEYKLVVVGAGDVGKSALTIQLIQNHFVDT |
| | 4xKRASmut_E7 | EYKLVVVGADGVGKSALTIQLIQNHFVDTE |
| | | YKLVVVGACGVGKSALTIQLIQNHFVDMHG |
| | | DTPTLHEYMLDLQPETTDLYCYEQLN |
| 6 | Amino acid | MTEYKLVVVGAVGVGKSALTIQLIQNHFVD |
| | suence of | TEYKLVVVGAGDVGKSALTIQLIQNHFVDT |
| | 4xKRASmut_ | EYKLVVVGADGVGKSALTIQLIQNHFVDTE |
| | EBV | YKLVVVGACGVGKSALTIQLIQNHFVDDAK |
| | | STKKCRAIVTDFSVIKAIEEEHRKKK |
| 7 | Nucleotide | ATGACGATACACTACAATTATATGTGCAAC |
| | sequence of | AGCAGCTGTATGGGCTCGATGAACAGAAGA |
| | 14xp53 | CCCATCCTGACAATCATTACCCTGGAGGAT |
| | (LCMV | TTGGATGACCGCAATACTTTCCGACACTCA |
| | backbone) | GTCGTAGTGCCGTGTGAACCCCCCGAGGTA |
| | | GGGAGCGACTGCACCACTATTCATTATTAT |
| | | AATTACATGTGTAACAGCAGTTGCATGGGT |
| | | GGTATGAACCAACGGCCCATCCTGACTATC |
| | | ATCACCCTTGAAGATTCAAGCGGGAACAGC |
| | | TTTGAGGTAAGAGTGTGTGCCTGCCCGGGA |
| | | AGAGACTGGCGCACGGAAGAAGAGAATCTG |
| | | AGAAAGAAGGGCGAGCCCCACCAGTCACAA |
| | | CATATGACAGAAGTAGTGAGAAGATGCCCT |
| | | AGAAGAGAGAGATGCAGTGATTCCGATGGC |
| | | CTGGCGCCACCACAACATGTAC |
| 8 | Nucleotide | AGTTATGGGTAGACTCCACGCCTCCCCCCG |
| | sequence of | GCACAAGATTCAGAGCCATGGCGATCTACA |
| | 14xp53_E7 | AACAGTCGCAACACATGACTGAGGATTCAA |
| | (LCMV | GCGGTAACCTTTTGGGCCGGAATAGCTTTG |
| | backbone) | AAGTGTGCGTTTGCGCGTGCCCCGGCAGAG |
| | | ATAGACGGACCGAGGAAGAGGGTAATCTGA |
| | | GAGTGGAATATCTGGATGATAGGAATACCT |
| | | TCCTCCACAGCGTCGTGGTCCCCTACGAAC |
| | | CCCCCGAGGTGGGGTCAGACTCCTCTGGCA |
| | | ACTTGCTGGGTCGGAACAGCTTCGAAGTGC |
| | | ACGTGTGCGCATGTCCTGGCAGAGATCGCC |
| | | GCACAGAGGAGGAGGACTCGAGCGGGAACC |
| | | TGCTCGGTCGGAACAGTTTCGAGGTGCTGG |
| | | TCTGCGCATGTCCCGGCAGAGACCGCAGAA |
| | | CCGAGGAGGAGGCGATTTATAAGCAATCGC |
| | | AACATATGACGGAGGTAGTGCGACACTGCC |
| | | CCCACCACGAGCGGTGTAGCGATAGTGATG |
| | | GGCTAGCTCAGCTGTGGGTGGATAGCACCC |
| | | CACCGCCCGGCACCCGCGTTCTGGCGATGG |
| | | CTATCTATAAACAATCTCAGCATATGACTG |
| | | AAGTGTGCTCCGACTCGGACGGACTTGCCC |
| | | CCCCTCAGCACTTAATCCCTGTGGAGGGGA |
| | | ACCTGAGGGTGGAGTATTTGGATGATCGCA |
| | | ATTACAACTATATGTGTAATTCTAGTTGTA |
| | | TGGGTGGAATGAACTGGAGGCCCATTCTAA |
| | | CCATTATCACACTCGAAGATTCTAGCGGCT |
| | | GAATGACGATACACTACAATTATATGTGCA |
| | | ACAGCAGCTGTATGGGCTCGATGAACAGAA |
| | | GACCCATCCTGACAATCATTACCCTGGAGG |
| | | ATTTGGATGACCGCAATACTTTCCGACACT |
| | | CAGTCGTAGTGCCGTGTGAACCCCCCGAGG |
| | | TAGGGAGCGACTGCACCACTATTCATTATT |
| | | ATAATTACATGTGTAACAGCAGTTGCATGG |
| | | GTGGTATGAACCAACGGCCCATCCTGACTA |
| | | TCATCACCCTTGAAGATTCAAGCGGGAACA |
| | | GCTTTGAGGTAAGAGTGTGTGCCTGCCCGG |
| | | GAAGAGACTGGCGCACGGAAGAAGAGAATC |
| | | TGAGAAAGAAGGGCGAGCCCCACCAGTCAC |
| | | AACATATGACAGAAGTAGTGAGAAGATGCC |
| | | CTAGAAGAGAGAGATGCAGTGATTCCGATG |
| | | GCCTGGCGCCACCACAACATGTACAGTTAT |
| | | GGGTAGACTCCACGCCTCCCCCCGGCACAA |
| | | GATTCAGAGCCATGGCGATCTACAAACAGT |
| | | CGCAACACATGACTGAGGATTCAAGCGGTA |
| | | ACCTTTTGGGCCGGAATAGCTTTGAAGTGT |
| | | GCGTTTGCGCGTGCCCCGGCAGAGATAGAC |
| | | GGACCGAGGAAGAGGGTAATCTGAGAGTGG |
| | | AATATCTGGATGATAGGAATACCTTCCTCC |
| | | ACAGCGTCGTGGTCCCCTACGAACCCCCCG |
| | | AGGTGGGGTCAGACTCCTCTGGCAACTTGC |
| | | TGGGTCGGAACAGCTTCGAAGTGCACGTGT |
| | | GCGCATGTCCTGGCAGAGATCGCCGCACAG |
| | | AGGAGGAGGACTCGAGCGGGAACCTGCTCG |
| | | GTCGGAACAGTTTCGAGGTGCTGGTCTGCG |
| | | CATGTCCCGGCAGAGACCGCAGAACCGAGG |
| | | AGGAGGCGATTTATAAGCAATCGCAACATA |
| | | TGACGGAGGTAGTGCGACACTGCCCCCACC |
| | | ACGAGCGGTGTAGCGATAGTGATGGGCTAG |
| | | CTCAGCTGTGGGTGGATAGCACCCCACCGC |
| | | CCGGCACCCGCGTTCTGGCGATGGCTATCT |
| | | ATAAACAATCTCAGCATATGACTGAAGTGT |
| | | GCTCCGACTCGGACGGACTTGCCCCCCCTC |
| | | AGCACTTAATCCCTGTGGAGGGGAACCTGA |
| | | GGGTGGAGTATTTGGATGATCGCAATTACA |
| | | ACTATATGTGTAATTCTAGTTGTATGGGTG |
| | | GAATGAACTGGAGGCCCATTCTAACCATTA |
| | | TCACACTCGAAGATTCTAGCGGCATGCATG |
| | | GGGACACCCCCACCCTGCATGAGTACATGC |
| | | TGGACCTCCAGCCTGAGACCACAGACCTGT |
| | | ACTGCTATGAGCAGCTGAACTGA |
| 9 | Nucleotide | ATGACGATACACTACAATTATATGTGCAAC |
| | sequence of | AGCAGCTGTATGGGCTCGATGAACAGAAGA |
| | 14xp53_EBV | CCCATCCTGACAATCATTACCCTGGAGGAT |
| | (LCMV | TTGGATGACCGCAATACTTTCCGACACTCA |
| | backbone) | GTCGTAGTGCCGTGTGAACCCCCCGAGGTA |
| | | GGGAGCGACTGCACCACTATTCATTATTAT |
| | | AATTACATGTGTAACAGCAGTTGCATGGGT |
| | | GGTATGAACCAACGGCCCATCCTGACTATC |
| | | ATCACCCTTGAAGATTCAAGCGGGAACAGC |
| | | TTTGAGGTAAGAGTGTGTGCCTGCCCGGGA |
| | | AGAGACTGGCGCACGGAAGAAGAGAATCTG |
| | | AGAAAGAAGGGCGAGCCCCACCAGTCACAA |
| | | CATATGACAGAAGTAGTGAGAAGATGCCCT |
| | | AGAAGAGAGAGATGCAGTGATTCCGATGGC |
| | | CTGGCGCCACCACAACATGTACAGTTATGG |
| | | GTAGACTCCACGCCTCCCCCCGGCACAAGA |
| | | TTCAGAGCCATGGCGATCTACAAACAGTCG |
| | | CAACACATGACTGAGGATTCAAGCGGTAAC |
| | | CTTTTGGGCCGGAATAGCTTTGAAGTGTGC |
| | | GTTTGCGCGTGCCCCGGCAGAGATAGACGG |
| | | ACCGAGGAAGAGGGTAATCTGAGAGTGGAA |
| | | TATCTGGATGATAGGAATACCTTCCTCCAC |
| | | AGCGTCGTGGTCCCCTACGAACCCCCCGAG |
| | | GTGGGGTCAGACTCCTCTGGCAACTTGCTG |
| | | GGTCGGAACAGCTTCGAAGTGCACGTGTGC |
| | | GCATGTCCTGGCAGAGATCGCCGCACAGAG |
| | | GAGGAGGACTCGAGCGGGAACCTGCTCGGT |
| | | CGGAACAGTTTCGAGGTGCTGGTCTGCGCA |
| | | TGTCCCGGCAGAGACCGCAGAACCGAGGAG |
| | | GAGGCGATTTATAAGCAATCGCAACATATG |
| | | ACGGAGGTAGTGCGACACTGCCCCCACCAC |
| | | GAGCGGTGTAGCGATAGTGATGGGCTAGCT |
| | | CAGCTGTGGGTGGATAGCACCCCACCGCCC |
| | | GGCACCCGCGTTCTGGCGATGGCTATCTAT |
| | | AAACAATCTCAGCATATGACTGAAGTGTGC |
| | | TCCGACTCGGACGGACTTGCCCCCCCTCAG |
| | | CACTTAATCCCTGTGGAGGGGAACCTGAGG |
| | | GTGGAGTATTTGGATGATCGCAATTACAAC |
| | | TATATGTGTAATTCTAGTTGTATGGGTGGA |
| | | ATGAACTGGAGGCCCATTCTAACCATTATC |
| | | ACACTCGAAGATTCTAGCGGCGATGCCAAG |
| | | AGCACCAAGAAGTGCAGAGCCATTGTGACT |
| | | GACTTCAGTGTGATCAAGGCCATAGAGGAG |
| | | GAGCACAGAAAGAAGAAGTGA |
| 10 | Nucleotide | ATGTTGGATGACCGCAATACTTTCCGACAC |
| | sequence of | TCAGTCGTAGTGCCGTGTGAACCCCCCGAG |
| | 14xp53 | GTAGGGAGCGACTGCACCACTATTCATTAT |
| | (PICV | TACAACTATATGTGTAATTCTAGTTGTATG |
| | backbone) | GGTGGAATGAACTGGAGGCCCATTCTAACC |
| | | ATTATCACACTCGAAGATTCTAGCGGCGAC |
| | | TCCTCTGGCAACTTGCTGGGTCGGAACAGC |
| | | TTCGAAGTGCACGTGTGCGCATGTCCTGGC |
| | | AGAGATCGCCGCACAGAGGAGGAGACGATA |
| | | CACTACAATTATATGTGCAACAGCAGCTGT |
| | | ATGGGCTCGATGAACAGAAGACCCATCCTG |
| | | ACAATCATTACCCTGGAGGATGACTCGAGC |
| | | GGGAACCTGCTCGGTCGGAACAGTTTCGAG |
| | | GTGCTGGTCTGCGCATGTCCCGGCAGAGAC |
| | | CGCAGAACCGAGGAGGAGTGCTCCGACTCG |
| | | GACGGACTTGCCCCCCCTCAGCACTTAATC |
| | | CCTGTGGAGGGGAACCTGAGGGTGGAGTAT |
| | | TTGGATGATCGCAATGTACAGTTATGGGTA |
| | | GACTCCACGCCTCCCCCCGGCACAAGATTC |
| | | AGAGCCATGGCGATCTACAAACAGTCGCAA |
| | | CACATGACTGAGGATTCAAGCGGTAACCTT |
| | | TTGGGCCGGAATAGCTTTGAAGTGTGCGTT |
| | | TGCGCGTGCCCCGGCAGAGATAGACGGACC |
| | | GAGGAAGAGCAGCTGTGGGTGGATAGCACC |
| | | CCACCGCCCGGCACCCGCGTTCTGGCGATG |
| | | GCTATCTATAAACAATCTCAGCATATGACT |
| | | GAAGTGAACAGCTTTGAGGTAAGAGTGTGT |
| | | GCCTGCCCGGGAAGAGACTGGCGCACGGAA |
| | | GAAGAGAATCTGAGAAAGAAGGGCGAGCCC |
| | | CACCAGTCACAACATATGACAGAAGTAGTG |
| | | AGAAGATGCCCTAGAAGAGAGAGATGCAGT |
| | | GATTCCGATGGCCTGGCGCCACCACAACAT |
| | | GGTAATCTGAGAGTGGAATATCTGGATGAT |
| | | AGGAATACCTTCCTCCACAGCGTCGTGGTC |
| | | CCCTACGAACCCCCCGAGGTGGGGTCAGCG |
| | | ATTTATAAGCAATCGCAACATATGACGGAG |
| | | GTAGTGCGACACTGCCCCCACCACGAGCGG |
| | | TGTAGCGATAGTGATGGGCTAGCTTATAAT |
| | | TACATGTGTAACAGCAGTTGCATGGGTGGT |
| | | ATGAACCAACGGCCCATCCTGACTATCATC |
| | | ACCCTTGAAGATTCAAGCGGGTGA |
| 11 | Nucleotide | ATGTTGGATGACCGCAATACTTTCCGACAC |
| | sequence of | TCAGTCGTAGTGCCGTGTGAACCCCCCGAG |
| | 14xp53_E7 | GTAGGGAGCGACTGCACCACTATTCATTAT |
| | (PICV | TACAACTATATGTGTAATTCTAGTTGTATG |
| | backbone) | GGTGGAATGAACTGGAGGCCCATTCTAACC |
| | | ATTATCACACTCGAAGATTCTAGCGGCGAC |
| | | TCCTCTGGCAACTTGCTGGGTCGGAACAGC |
| | | TTCGAAGTGCACGTGTGCGCATGTCCTGGC |
| | | AGAGATCGCCGCACAGAGGAGGAGACGATA |
| | | CACTACAATTATATGTGCAACAGCAGCTGT |
| | | ATGGGCTCGATGAACAGAAGACCCATCCTG |
| | | ACAATCATTACCCTGGAGGATGACTCGAGC |
| | | GGGAACCTGCTCGGTCGGAACAGTTTCGAG |
| | | GTGCTGGTCTGCGCATGTCCCGGCAGAGAC |
| | | CGCAGAACCGAGGAGGAGTGCTCCGACTCG |
| | | GACGGACTTGCCCCCCCTCAGCACTTAATC |
| | | CCTGTGGAGGGGAACCTGAGGGTGGAGTAT |
| | | TTGGATGATCGCAATGTACAGTTATGGGTA |
| | | GACTCCACGCCTCCCCCCGGCACAAGATTC |
| | | AGAGCCATGGCGATCTACAAACAGTCGCAA |
| | | CACATGACTGAGGATTCAAGCGGTAACCTT |
| | | TTGGGCCGGAATAGCTTTGAAGTGTGCGTT |
| | | TGCGCGTGCCCCGGCAGAGATAGACGGACC |
| | | GAGGAAGAGCAGCTGTGGGTGGATAGCACC |
| | | CCACCGCCCGGCACCCGCGTTCTGGCGATG |
| | | GCTATCTATAAACAATCTCAGCATATGACT |
| | | GAAGTGAACAGCTTTGAGGTAAGAGTGTGT |
| | | GCCTGCCCGGGAAGAGACTGGCGCACGGAA |
| | | GAAGAGAATCTGAGAAAGAAGGGCGAGCCC |
| | | CACCAGTCACAACATATGACAGAAGTAGTG |
| | | AGAAGATGCCCTAGAAGAGAGAGATGCAGT |
| | | GATTCCGATGGCCTGGCGCCACCACAACAT |
| | | GGTAATCTGAGAGTGGAATATCTGGATGAT |
| | | AGGAATACCTTCCTCCACAGCGTCGTGGTC |
| | | CCCTACGAACCCCCCGAGGTGGGGTCAGCG |
| | | ATTTATAAGCAATCGCAACATATGACGGAG |
| | | GTAGTGCGACACTGCCCCCACCACGAGCGG |
| | | TGTAGCGATAGTGATGGGCTAGCTTATAAT |
| | | TACATGTGTAACAGCAGTTGCATGGGTGGT |
| | | ATGAACCAACGGCCCATCCTGACTATCATC |
| | | ACCCTTGAAGATTCAAGCGGGATGCATGGG |
| | | GACACCCCCACCCTGCATGAGTACATGCTG |
| | | GACCTCCAGCCTGAGACCACAGACCTGTAC |
| | | TGCTATGAGCAGCTGAACTGA |
| 12 | Nucleotide | ATGTTGGATGACCGCAATACTTTCCGACAC |
| | sequence of | TCAGTCGTAGTGCCGTGTGAACCCCCCGAG |
| | 14xp53_EBV | GTAGGGAGCGACTGCACCACTATTCATTAT |
| | (PICV | TACAACTATATGTGTAATTCTAGTTGTATG |
| | backbone) | GGTGGAATGAACTGGAGGCCCATTCTAACC |
| | | ATTATCACACTCGAAGATTCTAGCGGCGAC |
| | | TCCTCTGGCAACTTGCTGGGTCGGAACAGC |
| | | TTCGAAGTGCACGTGTGCGCATGTCCTGGC |
| | | AGAGATCGCCGCACAGAGGAGGAGACGATA |
| | | CACTACAATTATATGTGCAACAGCAGCTGT |
| | | ATGGGCTCGATGAACAGAAGACCCATCCTG |
| | | ACAATCATTACCCTGGAGGATGACTCGAGC |
| | | GGGAACCTGCTCGGTCGGAACAGTTTCGAG |
| | | GTGCTGGTCTGCGCATGTCCCGGCAGAGAC |
| | | CGCAGAACCGAGGAGGAGTGCTCCGACTCG |
| | | GACGGACTTGCCCCCCCTCAGCACTTAATC |
| | | CCTGTGGAGGGGAACCTGAGGGTGGAGTAT |
| | | TTGGATGATCGCAATGTACAGTTATGGGTA |
| | | GACTCCACGCCTCCCCCCGGCACAAGATTC |
| | | AGAGCCATGGCGATCTACAAACAGTCGCAA |
| | | CACATGACTGAGGATTCAAGCGGTAACCTT |
| | | TTGGGCCGGAATAGCTTTGAAGTGTGCGTT |
| | | TGCGCGTGCCCCGGCAGAGATAGACGGACC |
| | | GAGGAAGAGCAGCTGTGGGTGGATAGCACC |
| | | CCACCGCCCGGCACCCGCGTTCTGGCGATG |
| | | GCTATCTATAAACAATCTCAGCATATGACT |
| | | GAAGTGAACAGCTTTGAGGTAAGAGTGTGT |
| | | GCCTGCCCGGGAAGAGACTGGCGCACGGAA |
| | | GAAGAGAATCTGAGAAAGAAGGGCGAGCCC |
| | | CACCAGTCACAACATATGACAGAAGTAGTG |
| | | AGAAGATGCCCTAGAAGAGAGAGATGCAGT |
| | | GATTCCGATGGCCTGGCGCCACCACAACAT |
| | | GGTAATCTGAGAGTGGAATATCTGGATGAT |
| | | AGGAATACCTTCCTCCACAGCGTCGTGGTC |
| | | CCCTACGAACCCCCCGAGGTGGGGTCAGCG |
| | | ATTTATAAGCAATCGCAACATATGACGGAG |
| | | GTAGTGCGACACTGCCCCCACCACGAGCGG |
| | | TGTAGCGATAGTGATGGGCTAGCTTATAAT |
| | | TACATGTGTAACAGCAGTTGCATGGGTGGT |
| | | ATGAACCAACGGCCCATCCTGACTATCATC |
| | | ACCCTTGAAGATTCAAGCGGGGATGCCAAG |
| | | AGCACCAAGAAGTGCAGAGCCATTGTGACT |
| | | GACTTCAGTGTGATCAAGGCCATAGAGGAG |
| | | GAGCACAGAAAGAAGAAGTGA |
| 13 | Amino acid | MTIHYNYMCNSSCMGSMNRRPILTIITLED |
| | suence of | LDDRNTFRHSVVVPCEPPEVGSDCTTIHYY |
| | 14xp53 | NYMCNSSCMGGMNQRPILTIITLEDSSGNS |
| | (LCMV | FEVRVCACPGRDWRTEEENLRKKGEPHQSQ |
| | backbone) | HMTEVVRRCPRRERCSDSDGLAPPQHVQLW |
| | | VDSTPPPGTRFRAMAIYKQSQHMTEDSSGN |
| | | LLGRNSFEVCVCACPGRDRRTEEEGNLRVE |
| | | YLDDRNTFLHSVVVPYEPPEVGSDSSGNLL |
| | | GRNSFEVHVCACPGRDRRTEEEDSSGNLLG |
| | | RNSFEVLVCACPGRDRRTEEEAIYKQSQHM |
| | | TEVVRHCPHHERCSDSDGLAQLWVDSTPPP |
| | | GTRVLAMAIYKQSQHMTEVCSDSDGLAPPQ |
| | | HLIPVEGNLRVEYLDDRNYNYMCNSSCMGG |
| | | MNWRPILTIITLEDSSG |
| 14 | Amino acid | MTIHYNYMCNSSCMGSMNRRPILTIITLED |
| | suence of | LDDRNTFRHSVVVPCEPPEVGSDCTTIHYY |
| | 14xp53_E7 | NYMCNSSCMGGMNQRPILTIITLEDSSGNS |
| | (LCMV | FEVRVCACPGRDWRTEEENLRKKGEPHQSQ |
| | backbone) | HMTEVVRRCPRRERCSDSDGLAPPQHVQLW |
| | | VDSTPPPGTRFRAMAIYKQSQHMTEDSSGN |
| | | LLGRNSFEVCVCACPGRDRRTEEEGNLRVE |
| | | YLDDRNTFLHSVVVPYEPPEVGSDSSGNLL |
| | | GRNSFEVHVCACPGRDRRTEEEDSSGNLLG |
| | | RNSFEVLVCACPGRDRRTEEEAIYKQSQHM |
| | | TEVVRHCPHHERCSDSDGLAQLWVDSTPPP |
| | | GTRVLAMAIYKQSQHMTEVCSDSDGLAPPQ |
| | | HLIPVEGNLRVEYLDDRNYNYMCNSSCMGG |
| | | MNWRPILTIITLEDSSGMHGDTPTLHEYML |
| | | DLQPETTDLYCYEQLN |
| 15 | Amino acid | MTIHYNYMCNSSCMGSMNRRPILTIITLED |
| | suence of | LDDRNTFRHSVVVPCEPPEVGSDCTTIHYY |
| | 14xp53_EBV | NYMCNSSCMGGMNQRPILTIITLEDSSGNS |
| | (LCMV | FEVRVCACPGRDWRTEEENLRKKGEPHQSQ |
| | backbone) | HMTEVVRRCPRRERCSDSDGLAPPQHVQLW |
| | | VDSTPPPGTRFRAMAIYKQSQHMTEDSSGN |
| | | LLGRNSFEVCVCACPGRDRRTEEEGNLRVE |
| | | YLDDRNTFLHSVVVPYEPPEVGSDSSGNLL |
| | | GRNSFEVHVCACPGRDRRTEEEDSSGNLLG |
| | | RNSFEVLVCACPGRDRRTEEEAIYKQSQHM |
| | | TEVVRHCPHHERCSDSDGLAQLWVDSTPPP |
| | | GTRVLAMAIYKQSQHMTEVCSDSDGLAPPQ |
| | | HLIPVEGNLRVEYLDDRNYNYMCNSSCMGG |
| | | MNWRPILTIITLEDSSGDAKSTKKCRAIVT |
| | | DFSVIKAIEEEHRKKK |
| 16 | Amino acid | MLDDRNTFRHSVVVPCEPPEVGSDCTTIHY |
| | suence of | YNYMCNSSCMGGMNWRPILTIITLEDSSGD |
| | 14xp53 | SSGNLLGRNSFEVHVCACPGRDRRTEEETI |
| | (PICV | HYNYMCNSSCMGSMNRRPILTIITLEDDSS |
| | backbone) | GNLLGRNSFEVLVCACPGRDRRTEEECSDS |
| | | DGLAPPQHLIPVEGNLRVEYLDDRNVQLWV |
| | | DSTPPPGTRFRAMAIYKQSQHMTEDSSGNL |
| | | LGRNSFEVCVCACPGRDRRTEEEQLWVDST |
| | | PPPGTRVLAMAIYKQSQHMTEVNSFEVRVC |
| | | ACPGRDWRTEEENLRKKGEPHQSQHMTEVV |
| | | RRCPRRERCSDSDGLAPPQHGNLRVEYLDD |
| | | RNTFLHSVVVPYEPPEVGSAIYKQSQHMTE |
| | | VVRHCPHHERCSDSDGLAYNYMCNSSCMGG |
| | | MNQRPILTIITLEDSSG |
| 17 | Amino acid | MLDDRNTFRHSVVVPCEPPEVGSDCTTIHY |
| | suence of | YNYMCNSSCMGGMNWRPILTIITLEDSSGD |
| | 14xp53_E7 | SSGNLLGRNSFEVHVCACPGRDRRTEEETI |
| | (PICV | HYNYMCNSSCMGSMNRRPILTIITLEDDSS |
| | backbone) | GNLLGRNSFEVLVCACPGRDRRTEEECSDS |
| | | DGLAPPQHLIPVEGNLRVEYLDDRNVQLWV |
| | | DSTPPPGTRFRAMAIYKQSQHMTEDSSGNL |
| | | LGRNSFEVCVCACPGRDRRTEEEQLWVDST |
| | | PPPGTRVLAMAIYKQSQHMTEVNSFEVRVC |
| | | ACPGRDWRTEEENLRKKGEPHQSQHMTEVV |
| | | RRCPRRERCSDSDGLAPPQHGNLRVEYLDD |
| | | RNTFLHSVVVPYEPPEVGSAIYKQSQHMTE |
| | | VVRHCPHHERCSDSDGLAYNYMCNSSCMGG |
| | | MNQRPILTIITLEDSSGMHGDTPTLHEYML |
| | | DLQPETTDLYCYEQLN |
| 18 | Amino acid | MLDDRNTFRHSVVVPCEPPEVGSDCTTIHY |
| | suence of | YNYMCNSSCMGGMNWRPILTIITLEDSSGD |
| | 14xp53_EBV | SSGNLLGRNSFEVHVCACPGRDRRTEEETI |
| | (PICV | HYNYMCNSSCMGSMNRRPILTIITLEDDSS |
| | backbone) | GNLLGRNSFEVLVCACPGRDRRTEEECSDS |
| | | DGLAPPQHLIPVEGNLRVEYLDDRNVQLWV |
| | | DSTPPPGTRFRAMAIYKQSQHMTEDSSGNL |
| | | LGRNSFEVCVCACPGRDRRTEEEQLWVDST |
| | | PPPGTRVLAMAIYKQSQHMTEVNSFEVRVC |
| | | ACPGRDWRTEEENLRKKGEPHQSQHMTEVV |
| | | RRCPRRERCSDSDGLAPPQHGNLRVEYLDD |
| | | RNTFLHSVVVPYEPPEVGSAIYKQSQHMTE |
| | | VVRHCPHHERCSDSDGLAYNYMCNSSCMGG |
| | | MNQRPILTIITLEDSSGDAKSTKKCRAIVT |
| | | DFSVIKAIEEEHRKKK |
| 19 | Amino acid | YKLVVVGAGDVGKSALTIYKLVVVGAVGVG |
| | suence of | KSALTIYKLVVVGACGVGKSALTIYKLVVV |
| | 5xKRASmut-H2 | GADGVGKSALTIYKLVVVGARGVGKSALTI |
| 20 | Nucleotide | ATGTACAAACTTGTTGTGGTGGGAGCTGGG |
| | sequence of | GATGTGGGAAAGTCAGCCCTCACAATCTAC |
| | 5xKRASmut-H2 | AAATTGGTGGTTGTGGGTGCAGTTGGTGTT |
| | | GGCAAATCAGCCCTCACCATCTACAAACTT |
| | | GTTGTAGTTGGTGCCTGTGGAGTAGGGAAA |
| | | TCAGCCCTCACCATCTACAAGCTGGTTGTG |
| | | GTTGGTGCTGATGGAGTGGGAAAGTCAGCC |
| | | CTCACCATCTACAAGTTGGTGGTTGTAGGT |
| | | GCCAGAGGGGTGGGAAAATCTGCCCTGACC |
| | | ATCTGA |
| 21 | Nucleotide | GCGCACCGGGGATCCTAGGCTTTTTGGATT |
| | sequence of | GCGCTTTCCTCTAGATCAACTGGGTGTCAG |
| | artLCMV- | GCCCTATCCTACAGAAGGATGTACAAACTT |
| | 5xKRASmut- | GTTGTGGTGGGAGCTGGGGATGTGGGAAAG |
| | H2-NP-S- | TCAGCCCTCACAATCTACAAATTGGTGGTT |
| | segment. | GTGGGTGCAGTTGGTGTTGGCAAATCAGCC |
| | | CTCACCATCTACAAACTTGTTGTAGTTGGT |
| | | GCCTGTGGAGTAGGGAAATCAGCCCTCACC |
| | | ATCTACAAGCTGGTTGTGGTTGGTGCTGAT |
| | | GGAGTGGGAAAGTCAGCCCTCACCATCTAC |
| | | AAGTTGGTGGTTGTAGGTGCCAGAGGGGTG |
| | | GGAAAATCTGCCCTGACCATCTGAAGAACA |
| | | GCGCCTCCCTGACTCTCCACCTCGAAAGAG |
| | | GTGGAGAGTCAGGGAGGCCCAGAGGGTCTT |
| | | AGAGTGTCACAACATTTGGGCCTCTAAAAA |
| | | TTAGGTCATGTGGCAGAATGTTGTGAACAG |
| | | TTTTCAGATCTGGGAGCCTTGCTTTGGAGG |
| | | CGCTTTCAAAAATGATGCAGTCCATGAGTG |
| | | CACAGTGCGGGGTGATCTCTTTCTTCTTTT |
| | | TGTCCCTTACTATTCCAGTATGCATCTTAC |
| | | ACAACCAGCCATATTTGTCCCACACTTTAT |
| | | CTTCATACTCCCTCGAAGCTTCCCTGGTCA |
| | | TTTCAACATCGATAAGCTTAATGTCCTTCC |
| | | TATTTTGTGAGTCCAGAAGCTTTCTGATGT |
| | | CATCGGAGCCTTGACAGCTTAGAACCATCC |
| | | CCTGCGGAAGAGCACCTATAACTGACGAGG |
| | | TCAACCCGGGTTGCGCATTGAAGAGGTCGG |
| | | CAAGATCCATGCCGTGTGAGTACTTGGAAT |
| | | CTTGCTTGAATTGTTTTTGATCAACGGGTT |
| | | CCCTGTAAAAGTGTATGAACTGCCCGTTCT |
| | | GTGGTTGGAAAATTGCTATTTCCACTGGAT |
| | | CATTAAATCTACCCTCAATGTCAATCCATG |
| | | TAGGAGCGTTGGGGTCAATTCCTCCCATGA |
| | | GGTCTTTTAAAAGCATTGTCTGGCTGTAGC |
| | | TTAAGCCCACCTGAGGTGGACCTGCTGCTC |
| | | CAGGCGCTGGCCTGGGTGAGTTGACTGCAG |
| | | GTTTCTCGCTTGTGAGATCAATTGTTGTGT |
| | | TTTCCCATGCTCTCCCCACAATCGATGTTC |
| | | TACAAGCTATGTATGGCCATCCTTCACCTG |
| | | AAAGGCAAACTTTATAGAGGATGTTTTCAT |
| | | AAGGGTTCCTGTCCCCAACTTGGTCTGAAA |
| | | CAAACATGTTGAGTTTTCTCTTGGCCCCGA |
| | | GAACTGCCTTCAAGAGATCCTCGCTGTTGC |
| | | TTGGCTTGATCAAAATTGACTCTAACATGT |
| | | TACCCCCATCCAACAGGGCTGCCCCTGCCT |
| | | TCACGGCAGCACCAAGACTAAAGTTATAGC |
| | | CAGAAATGTTGATGCTGGACTGCTGTTCAG |
| | | TGATGACCCCCAGAACTGGGTGCTTGTCTT |
| | | TCAGCCTTTCAAGATCATTAAGATTTGGAT |
| | | ACTTGACTGTGTAAAGCAAGCCAAGGTCTG |
| | | TGAGCGCTTGTACAACGTCATTGAGCGGAG |
| | | TCTGTGACTGTTTGGCCATACAAGCCATAG |
| | | TTAGACTTGGCATTGTGCCAAATTGATTGT |
| | | TCAAAAGTGATGAGTCTTTCACATCCCAAA |
| | | CTCTTACCACACCACTTGCACCCTGCTGAG |
| | | GCTTTCTCATCCCAACTATCTGTAGGATCT |
| | | GAGATCTTTGGTCTAGTTGCTGTGTTGTTA |
| | | AGTTCCCCATATATACCCCTGAAGCCTGGG |
| | | GCCTTTCAGACCTCATGATCTTGGCCTTCA |
| | | GCTTCTCAAGGTCAGCCGCAAGAGACATCA |
| | | GTTCTTCTGCACTGAGCCTCCCCACTTTCA |
| | | AAACATTCTTCTTTGATGTTGACTTTAAAT |
| | | CCACAAGAGAATGTACAGTCTGGTTGAGAC |
| | | TTCTGAGTCTCTGTAGGTCTTTGTCATCTC |
| | | TCTTTTCCTTCCTCATGATCCTCTGAACAT |
| | | TGCTGACCTCAGAGAAGTCCAACCCATTCA |
| | | GAAGGTTGGTTGCATCCTTAATGACAGCAG |
| | | CCTTCACATCTGATGTGAAGCTCTGCAATT |
| | | CTCTTCTCAATGCTTGCGTCCATTGGAAGC |
| | | TCTTAACTTCCTTAGACAAGGACATCTTGT |
| | | TGCTCAATGGTTTCTCAAGACAAATGCGCA |
| | | ATCAAATGCCTAGGATCCACTGTGCG |
| 22 | Nucleotide | GCGCACCGGGGATCCTAGGCTTTTTGGATT |
| | sequence of | GCGCTTTCCTCTAGATCAACTGGGTGTCAG |
| | artLCMV- | GCCCTATCCTACAGAAGGATGTACAAACTT |
| | 5xKRASmut-H2- | GTTGTGGTGGGAGCTGGGGATGTGGGAAAG |
| | GP-S-segment | TCAGCCCTCACAATCTACAAATTGGTGGTT |
| | | GTGGGTGCAGTTGGTGTTGGCAAATCAGCC |
| | | CTCACCATCTACAAACTTGTTGTAGTTGGT |
| | | GCCTGTGGAGTAGGGAAATCAGCCCTCACC |
| | | ATCTACAAGCTGGTTGTGGTTGGTGCTGAT |
| | | GGAGTGGGAAAGTCAGCCCTCACCATCTAC |
| | | AAGTTGGTGGTTGTAGGTGCCAGAGGGGTG |
| | | GGAAAATCTGCCCTGACCATCTGAAGAACA |
| | | GCGCCTCCCTGACTCTCCACCTCGAAAGAG |
| | | GTGGAGAGTCAGGGAGGCCCAGAGGGTCTC |
| | | AGCGTCTTTTCCAGATAGTTTTTACACCAG |
| | | GCACCTTGAATGCACCACAACTACAGATCC |
| | | CCTTGTTGGTCAAGCGGTGTGGCTTTGGAC |
| | | ATGAACCGCCCTTTATGTGTCTATGTGTTG |
| | | GTATCTTCACAAGATGCAGAAAGATGCTGA |
| | | TTAGATATGCTGATGTTGAAAACATCAAAA |
| | | GATCCATTAAGGCTAAAGGAGTACTCCCTT |
| | | GTCTTTTTATGTAGTCCTTCCTCAACATCT |
| | | CTGTGATCATGTTATCTGCTTCTTGTTCGA |
| | | TTTGATCACTAAAGTGGGTCTCATTCAAGT |
| | | AGGAGCCATTAGTGACAAGCCAGCACTTGG |
| | | GTACACTAGTCTCACCAGTCTTAGCATGTT |
| | | CCAGATACCAGAACTTTGAGTAATTACAGT |
| | | ATGGTACCCCCATTAGATCTCTTAGATGAT |
| | | TCCTCATCAACAGCTGATCGGAAATCAGAG |
| | | AATTTACTGTTGTTTTGAATACATGCAAGG |
| | | CAGACTCTACATCTTGCTTGAACTTACTCA |
| | | GGGCGGCCTTGTTGTAATCAATTAGTCGTA |
| | | GCATGTCACAGAACTCTTCATCATGATTGA |
| | | CATTACATTTTGCAACAGCTGTATTCCCAA |
| | | AACATTTGAGCTCTGCAGCAAGGATCATCC |
| | | ATTTGGTCAGGCAATAACCACCTGGATTTT |
| | | CTACTCCTGAGGAGTCTGACAGGGTCCAGG |
| | | TGAATGTGCCTGCAAGTCTCCTAGTGAGAA |
| | | ACTTTGTCTTTTCCTGAGCAAAGAGGATTC |
| | | TAGACATCCCAAAAGGGCCTGCATATCTAC |
| | | AGTGGTTTTCCCAAGTCCTGTTTTGTATGA |
| | | TTAGGTACTGATAGCTTGTTTGGCTGCACC |
| | | AAGTGGTCTTGCCATCTGAACCTGCCCAGC |
| | | CCCAGCCACTTCTCATGTATTTTCCTCCAA |
| | | AGGCAGTTCTAAACATGTCCAAGACTCTAC |
| | | CTCTGAAAGTCCTACACTGGCTTATAGCGC |
| | | TCTGTGGGTCCGAAAATGACAAGTTGTATT |
| | | GAATGGTGATGCCATTGTTAAAATCACAAG |
| | | ACACTGCTTTGTGGTTGGAATTCCCTCTAA |
| | | TACTGAGGTGCAGACTCGAGACTATACTCA |
| | | TGAGTGTATGGTCAAAAGTCTTTTTGTTGA |
| | | AAGCGGAGGTTAAGTTGCAAAAATTGTGAT |
| | | TAAGGATGGAGTCGTTAGTGAAAGTTAGCT |
| | | CCAGTCCAGAGCTTCCCATACTGATGTAGT |
| | | GATGAGAGTTGTTGGCTGAGCACGCATTGG |
| | | GCATCGTCAGATTTAAGTGAGACATATCAA |
| | | ACTCCACTGATTTGAACTGGTAAACCCCTT |
| | | TATAGATGTCGGGACCATTAAGGCCGTACA |
| | | TGCCACAGGACCTACCAGCCAAAAAAAGGA |
| | | AGCTGACCAGTGCTAATATCCCACAGGTGG |
| | | CGAAATTGTACACAGCTTTGATGCTCGTGA |
| | | TTATAATGAGCACAATAATGACAATGTTGA |
| | | TGACCTCATCAATGATGTGAGGCAAAGCCT |
| | | CAAACATTGTCACAATCTGACCCATCTTGT |
| | | TGCTCAATGGTTTCTCAAGACAAATGCGCA |
| | | ATCAAATGCCTAGGATCCACTGTGCG |
| 23 | Nucleotide | GCGCACCGGGGATCCTAGGCATACCTTGGA |
| | sequence of | CGCGCATATTACTTGATCAAAGATGTACAA |
| | artPICV- | ACTTGTTGTGGTGGGAGCTGGGGATGTGGG |
| | 5xKRASmut-H2- | AAAGTCAGCCCTCACAATCTACAAATTGGT |
| | NP-S-segment | GGTTGTGGGTGCAGTTGGTGTTGGCAAATC |
| | | AGCCCTCACCATCTACAAACTTGTTGTAGT |
| | | TGGTGCCTGTGGAGTAGGGAAATCAGCCCT |
| | | CACCATCTACAAGCTGGTTGTGGTTGGTGC |
| | | TGATGGAGTGGGAAAGTCAGCCCTCACCAT |
| | | CTACAAGTTGGTGGTTGTAGGTGCCAGAGG |
| | | GGTGGGAAAATCTGCCCTGACCATCTGAGC |
| | | CCTAGCCTCGACATGGGCCTCGACGTCACT |
| | | CCCCAATAGGGGAGTGACGTCGAGGCCTCT |
| | | GAGGACTTGAGCTCAGAGGTTGATCAGATC |
| | | TGTGTTGTTCCTGTACAGCGTGTCAATAGG |
| | | CAAGCATCTCATCGGCTTCTGGTCCCTAAC |
| | | CCAGCCTGTCACTGTTGCATCAAACATGAT |
| | | GGTATCAAGCAATGCACAGTGAGGATTCGC |
| | | AGTGGTTTGTGCAGCCCCCTTCTTCTTCTT |
| | | CTTTATGACCAAACCTTTATGTTTGGTGCA |
| | | GAGTAGATTGTATCTCTCCCAGATCTCATC |
| | | CTCAAAGGTGCGTGCTTGCTCGGCACTGAG |
| | | TTTCACGTCAAGCACTTTTAAGTCTCTTCT |
| | | CCCATGCATTTCGAACAAACTGATTATATC |
| | | ATCTGAACCTTGAGCAGTGAAAACCATGTT |
| | | TTGAGGTAAATGTCTGATGATTGAGGAAAT |
| | | CAGGCCTGGTTGGGCATCAGCCAAGTCCTT |
| | | TAAAAGGAGACCATGTGAGTACTTGCTTTG |
| | | CTCTTTGAAGGACTTCTCATCGTGGGGAAA |
| | | TCTGTAACAATGTATGTAGTTGCCCGTGTC |
| | | AGGCTGGTAGATGGCCATTTCCACCGGATC |
| | | ATTTGGTGTTCCTTCAATGTCAATCCATGT |
| | | GGTAGCTTTTGAATCAAGCATCTGAATTGA |
| | | GGACACAACAGTATCTTCTTTCTCCTTAGG |
| | | GATTTGTTTAAGGTCCGGTGATCCTCCGTT |
| | | TCTTACTGGTGGCTGGATAGCACTCGGCTT |
| | | CGAATCTAAATCTACAGTGGTGTTATCCCA |
| | | AGCCCTCCCTTGAACTTGAGACCTTGAGCC |
| | | AATGTAAGGCCAACCATCCCCTGAAAGACA |
| | | AATCTTGTATAGTAAATTTTCATAAGGATT |
| | | TCTCTGTCCGGGTGTAGTGCTCACAAACAT |
| | | ACCTTCACGATTCTTTATTTGCAATAGACT |
| | | CTTTATGAGAGTACTAAACATAGAAGGCTT |
| | | CACCTGGATGGTCTCAAGCATATTGCCACC |
| | | ATCAATCATGCAAGCAGCTGCTTTGACTGC |
| | | TGCAGACAAACTGAGATTGTACCCTGAGAT |
| | | GTTTATGGCTGATGGCTCATTACTAATGAT |
| | | TTTTAGGGCACTGTGTTGCTGTGTGAGTTT |
| | | CTCTAGATCTGTCATGTTCGGGAACTTGAC |
| | | AGTGTAGAGCAAACCAAGTGCACTCAGCGC |
| | | TTGGACAACATCATTAAGTTGTTCACCCCC |
| | | TTGCTCAGTCATACAAGCGATGGTTAAGGC |
| | | TGGCATTGATCCAAATTGATTGATCAACAA |
| | | TGTATTATCCTTGATGTCCCAGATCTTCAC |
| | | AACCCCATCTCTGTTGCCTGTGGGTCTAGC |
| | | ATTAGCGAACCCCATTGAGCGAAGGATTTC |
| | | GGCTCTTTGTTCCAACTGAGTGTTTGTGAG |
| | | ATTGCCCCCATAAACACCAGGCTGAGACAA |
| | | ACTCTCAGTTCTAGTGACTTTCTTTCTTAA |
| | | CTTGTCCAAATCAGATGCAAGCTCCATTAG |
| | | CTCCTCTTTGGCTAAGCCTCCCACCTTAAG |
| | | CACATTGTCCCTCTGGATTGATCTCATATT |
| | | CATCAGAGCATCAACCTCTTTGTTCATGTC |
| | | TCTTAACTTGGTCAGATCAGAATCAGTCCT |
| | | TTTATCTTTGCGCATCATTCTTTGAACTTG |
| | | AGCAACTTTGTGAAAGTCAAGAGCAGATAA |
| | | CAGTGCTCTTGTGTCCGACAACACATCAGC |
| | | CTTCACAGGATGGGTCCAGTTGGATAGACC |
| | | CCTCCTAAGGGACTGTACCCAGCGGAATGA |
| | | TGGGATGTTGTCAGACATTTTGGGGTTGTT |
| | | TGCACTTCCTCCGAGTCAGTGAAGAAGTGA |
| | | ACGTACAGCGTGATCTAGAATCGCCTAGGA |
| | | TCCACTGTGCG |
| 24 | Nucleotide | GCGCACCGGGGATCCTAGGCATACCTTGGA |
| | sequence of | CGCGCATATTACTTGATCAAAGATGTACAA |
| | artPCIV- | ACTTGTTGTGGTGGGAGCTGGGGATGTGGG |
| | 5xKRASmut-H2- | AAAGTCAGCCCTCACAATCTACAAATTGGT |
| | GP-S-segment | GGTTGTGGGTGCAGTTGGTGTTGGCAAATC |
| | | AGCCCTCACCATCTACAAACTTGTTGTAGT |
| | | TGGTGCCTGTGGAGTAGGGAAATCAGCCCT |
| | | CACCATCTACAAGCTGGTTGTGGTTGGTGC |
| | | TGATGGAGTGGGAAAGTCAGCCCTCACCAT |
| | | CTACAAGTTGGTGGTTGTAGGTGCCAGAGG |
| | | GGTGGGAAAATCTGCCCTGACCATCTGAGC |
| | | CCTAGCCTCGACATGGGCCTCGACGTCACT |
| | | CCCCAATAGGGGAGTGACGTCGAGGCCTCT |
| | | GAGGACTTGAGCTTATTTACCCAGTCTCAC |
| | | CCATTTGTAGGGTTTCTTTGGGATTTTATA |
| | | ATACCCACAGCTGCAAAGAGAGTTCCTAGT |
| | | AATCCTATGTGGCTTCGGACAGCCATCACC |
| | | AATGATGTGCCTATGAGTGGGTATTCCAAC |
| | | TAAGTGGAGAAACACTGTGATGGTGTAAAA |
| | | CACCAAAGACCAGAAGCAAATGTCTGTCAA |
| | | TGCTAGTGGAGTCTTACCTTGTCTTTCTTC |
| | | ATATTCTTTTATCAGCATTTCATTGTACAG |
| | | ATTCTGGCTCTCCCACAACCAATCATTCTT |
| | | AAAATGCGTTTCATTGAGGTACGAGCCATT |
| | | GTGAACTAACCAACACTGCGGTAAAGAATG |
| | | TCTCCCTGTGATGGTATCATTGATGTACCA |
| | | AAATTTTGTATAGTTGCAATAAGGGATTTT |
| | | GGCAAGCTGTTTGAGACTGTTTCTAATCAC |
| | | AAGTGAGTCAGAAATAAGTCCGTTGATAGT |
| | | CTTTTTAAAGAGATTCAACGAATTCTCAAC |
| | | ATTAAGTTGTAAGGTTTTGATAGCATTCTG |
| | | ATTGAAATCAAATAACCTCATCGTATCGCA |
| | | AAATTCTTCATTGTGATCTTTGTTGCATTT |
| | | TGCCATCACAGTGTTATCAAAACATTTTAT |
| | | TCCAGCCCAAACAATAGCCCATTGCTCCAA |
| | | ACAGTAACCACCTGGGACATGTTGCCCAGT |
| | | AGAGTCACTCAAGTCCCAAGTGAAAAAGCC |
| | | AAGGAGTTTCCTGCTCACAGAACTATAAGC |
| | | AGTTTTTTGGAGAGCCATCCTTATTGTTGC |
| | | CATTGGAGTATATGTACAGTGATTTTCCCA |
| | | TGTGGTGTTCTGTATGATCAGGAAATTGTA |
| | | ATGTGTCCCACCTTCACAGTTTGTTAGTCT |
| | | GCAAGACCCTCCACTACAGTTATTGAAACA |
| | | TTTTCCAACCCACGCAATTTTTGGGTCCCC |
| | | AATGATTTGAGCAAGCGACGCAATAAGATG |
| | | TCTGCCAACCTCACCTCCTCTATCCCCAAC |
| | | TGTCAAGTTGTACTGGATCAACACCCCAGC |
| | | ACCCTCAACTGTTTTGCATCTGGCACCTAC |
| | | ATGACGAGTGACATGGAGCACATTGAAGTG |
| | | TAACTCATTAAGCAACCATTTTAATGTGTG |
| | | ACCTGCTTCTTCTGTCTTATCACAATTACT |
| | | AATGTTACCATATGCAAGGCTTCTGATGTT |
| | | GGAAAAGTTTCCAGTAGTTTCATTTGCAAT |
| | | GGATGTGTTTGTCAAAGTGAGTTCAATTCC |
| | | CCATGTTGTGTTAGATGGTCCTTTGTAGTA |
| | | ATGATGTGTGTTGTTCTTGCTACATGATTG |
| | | TGGCAAGTTGTCAAACATTCTTGTGAGGTT |
| | | GAACTCAACGTGGGTGAGATTGTGCCTCCT |
| | | ATCAATCATCATGCCATCACAACTTCTGCC |
| | | AGCCAAAATGAGGAAGGTGATGAGTTGGAA |
| | | TAGGCCACATCTCATCAGATTGACAAATCC |
| | | TTTGATGATGCATAGGGTTGAGACAATGAT |
| | | TAAGGCGACATTGAACACCTCCTGCAGGAC |
| | | TTCGGGTATAGACTGGATCAAAGTCACAAC |
| | | TTGTCCCATTTTGGGGTTGTTTGCACTTCC |
| | | TCCGAGTCAGTGAAGAAGTGAACGTACAGC |
| | | GTGATCTAGAATCGCCTAGGATCCACTGTG |
| | | CG |
| 25 | Amino acid | YKLVVVGAVGVGKSALTIGGSGGGGSGGYK |
| | suence of | LVVVGARGVGKSALTIGGSGGGGSGGYKLV |
| | 5xKRASmut-H1 | VVGACGVGKSALTIGGSGGGGSGGYKLVVV |
| | | GAGDVGKSALTIGGSGGGGSGGYKLVVVGA |
| | | DGVGKSALTI |
| 26 | Nucleotide | ATGTACAAGCTGGTGGTAGTTGGAGCAGTG |
| | sequence of | GGAGTGGGCAAATCAGCCTTGACCATTGGA |
| | 5xKRASmut-H1 | GGCTCAGGTGGTGGAGGCTCAGGAGGCTAC |
| | | AAACTTGTGGTGGTTGGAGCCAGAGGTGTA |
| | | GGCAAAAGTGCACTCACCATAGGAGGAAGT |
| | | GGAGGCGGAGGTAGTGGAGGCTACAAACTT |
| | | GTTGTTGTTGGGGCCTGTGGGGTGGGAAAG |
| | | TCAGCACTCACCATTGGTGGCAGTGGAGGT |
| | | GGAGGCAGTGGTGGCTACAAGTTGGTGGTC |
| | | GTAGGTGCTGGGGATGTGGGCAAATCTGCC |
| | | CTCACAATTGGAGGCTCAGGAGGTGGAGGC |
| | | AGTGGTGGCTACAAATTGGTTGTGGTGGGT |
| | | GCAGATGGTGTGGGCAAAAGTGCTTTGACC |
| | | ATCTGA |
| 27 | Nucleotide | GCGCACCGGGGATCCTAGGCTTTTTGGATT |
| | sequence of | GCGCTTTCCTCTAGATCAACTGGGTGTCAG |
| | artLCMV- | GCCCTATCCTACAGAAGGATGTACAAGCTG |
| | 5xKRASmut-H1- | GTGGTAGTTGGAGCAGTGGGAGTGGGCAAA |
| | NP-S-segment | TCAGCCTTGACCATTGGAGGCTCAGGTGGT |
| | | GGAGGCTCAGGAGGCTACAAACTTGTGGTG |
| | | GTTGGAGCCAGAGGTGTAGGCAAAAGTGCA |
| | | CTCACCATAGGAGGAAGTGGAGGCGGAGGT |
| | | AGTGGAGGCTACAAACTTGTTGTTGTTGGG |
| | | GCCTGTGGGGTGGGAAAGTCAGCACTCACC |
| | | ATTGGTGGCAGTGGAGGTGGAGGCAGTGGT |
| | | GGCTACAAGTTGGTGGTCGTAGGTGCTGGG |
| | | GATGTGGGCAAATCTGCCCTCACAATTGGA |
| | | GGCTCAGGAGGTGGAGGCAGTGGTGGCTAC |
| | | AAATTGGTTGTGGTGGGTGCAGATGGTGTG |
| | | GGCAAAAGTGCTTTGACCATCTGAAGAACA |
| | | GCGCCTCCCTGACTCTCCACCTCGAAAGAG |
| | | GTGGAGAGTCAGGGAGGCCCAGAGGGTCTT |
| | | AGAGTGTCACAACATTTGGGCCTCTAAAAA |
| | | TTAGGTCATGTGGCAGAATGTTGTGAACAG |
| | | TTTTCAGATCTGGGAGCCTTGCTTTGGAGG |
| | | CGCTTTCAAAAATGATGCAGTCCATGAGTG |
| | | CACAGTGCGGGGTGATCTCTTTCTTCTTTT |
| | | TGTCCCTTACTATTCCAGTATGCATCTTAC |
| | | ACAACCAGCCATATTTGTCCCACACTTTAT |
| | | CTTCATACTCCCTCGAAGCTTCCCTGGTCA |
| | | TTTCAACATCGATAAGCTTAATGTCCTTCC |
| | | TATTTTGTGAGTCCAGAAGCTTTCTGATGT |
| | | CATCGGAGCCTTGACAGCTTAGAACCATCC |
| | | CCTGCGGAAGAGCACCTATAACTGACGAGG |
| | | TCAACCCGGGTTGCGCATTGAAGAGGTCGG |
| | | CAAGATCCATGCCGTGTGAGTACTTGGAAT |
| | | CTTGCTTGAATTGTTTTTGATCAACGGGTT |
| | | CCCTGTAAAAGTGTATGAACTGCCCGTTCT |
| | | GTGGTTGGAAAATTGCTATTTCCACTGGAT |
| | | CATTAAATCTACCCTCAATGTCAATCCATG |
| | | TAGGAGCGTTGGGGTCAATTCCTCCCATGA |
| | | GGTCTTTTAAAAGCATTGTCTGGCTGTAGC |
| | | TTAAGCCCACCTGAGGTGGACCTGCTGCTC |
| | | CAGGCGCTGGCCTGGGTGAGTTGACTGCAG |
| | | GTTTCTCGCTTGTGAGATCAATTGTTGTGT |
| | | TTTCCCATGCTCTCCCCACAATCGATGTTC |
| | | TACAAGCTATGTATGGCCATCCTTCACCTG |
| | | AAAGGCAAACTTTATAGAGGATGTTTTCAT |
| | | AAGGGTTCCTGTCCCCAACTTGGTCTGAAA |
| | | CAAACATGTTGAGTTTTCTCTTGGCCCCGA |
| | | GAACTGCCTTCAAGAGATCCTCGCTGTTGC |
| | | TTGGCTTGATCAAAATTGACTCTAACATGT |
| | | TACCCCCATCCAACAGGGCTGCCCCTGCCT |
| | | TCACGGCAGCACCAAGACTAAAGTTATAGC |
| | | CAGAAATGTTGATGCTGGACTGCTGTTCAG |
| | | TGATGACCCCCAGAACTGGGTGCTTGTCTT |
| | | TCAGCCTTTCAAGATCATTAAGATTTGGAT |
| | | ACTTGACTGTGTAAAGCAAGCCAAGGTCTG |
| | | TGAGCGCTTGTACAACGTCATTGAGCGGAG |
| | | TCTGTGACTGTTTGGCCATACAAGCCATAG |
| | | TTAGACTTGGCATTGTGCCAAATTGATTGT |
| | | TCAAAAGTGATGAGTCTTTCACATCCCAAA |
| | | CTCTTACCACACCACTTGCACCCTGCTGAG |
| | | GCTTTCTCATCCCAACTATCTGTAGGATCT |
| | | GAGATCTTTGGTCTAGTTGCTGTGTTGTTA |
| | | AGTTCCCCATATATACCCCTGAAGCCTGGG |
| | | GC |
| 28 | Nucleotide | CTTTCAGACCTCATGATCTTGGCCTTCAGC |
| | sequence of | TTCTCAAGGTCAGCCGCAAGAGACATCAGT |
| | artLCMV- | TCTTCTGCACTGAGCCTCCCCACTTTCAAA |
| | 5xKRASmut- | ACATTCTTCTTTGATGTTGACTTTAAATCC |
| | H1-GP-S- | ACAAGAGAATGTACAGTCTGGTTGAGACTT |
| | segment | CTGAGTCTCTGTAGGTCTTTGTCATCTCTC |
| | | TTTTCCTTCCTCATGATCCTCTGAACATTG |
| | | CTGACCTCAGAGAAGTCCAACCCATTCAGA |
| | | AGGTTGGTTGCATCCTTAATGACAGCAGCC |
| | | TTCACATCTGATGTGAAGCTCTGCAATTCT |
| | | CTTCTCAATGCTTGCGTCCATTGGAAGCTC |
| | | TTAACTTCCTTAGACAAGGACATCTTGTTG |
| | | CTCAATGGTTTCTCAAGACAAATGCGCAAT |
| | | CAAATGCCTAGGATCCACTGTGCGGCGCAC |
| | | CGGGGATCCTAGGCTTTTTGGATTGCGCTT |
| | | TCCTCTAGATCAACTGGGTGTCAGGCCCTA |
| | | TCCTACAGAAGGATGTACAAGCTGGTGGTA |
| | | GTTGGAGCAGTGGGAGTGGGCAAATCAGCC |
| | | TTGACCATTGGAGGCTCAGGTGGTGGAGGC |
| | | TCAGGAGGCTACAAACTTGTGGTGGTTGGA |
| | | GCCAGAGGTGTAGGCAAAAGTGCACTCACC |
| | | ATAGGAGGAAGTGGAGGCGGAGGTAGTGGA |
| | | GGCTACAAACTTGTTGTTGTTGGGGCCTGT |
| | | GGGGTGGGAAAGTCAGCACTCACCATTGGT |
| | | GGCAGTGGAGGTGGAGGCAGTGGTGGCTAC |
| | | AAGTTGGTGGTCGTAGGTGCTGGGGATGTG |
| | | GGCAAATCTGCCCTCACAATTGGAGGCTCA |
| | | GGAGGTGGAGGCAGTGGTGGCTACAAATTG |
| | | GTTGTGGTGGGTGCAGATGGTGTGGGCAAA |
| | | AGTGCTTTGACCATCTGAAGAACAGCGCCT |
| | | CCCTGACTCTCCACCTCGAAAGAGGTGGAG |
| | | AGTCAGGGAGGCCCAGAGGGTCTCAGCGTC |
| | | TTTTCCAGATAGTTTTTACACCAGGCACCT |
| | | TGAATGCACCACAACTACAGATCCCCTTGT |
| | | TGGTCAAGCGGTGTGGCTTTGGACATGAAC |
| | | CGCCCTTTATGTGTCTATGTGTTGGTATCT |
| | | TCACAAGATGCAGAAAGATGCTGATTAGAT |
| | | ATGCTGATGTTGAAAACATCAAAAGATCCA |
| | | TTAAGGCTAAAGGAGTACTCCCTTGTCTTT |
| | | TTATGTAGTCCTTCCTCAACATCTCTGTGA |
| | | TCATGTTATCTGCTTCTTGTTCGATTTGAT |
| | | CACTAAAGTGGGTCTCATTCAAGTAGGAGC |
| | | CATTAGTGACAAGCCAGCACTTGGGTACAC |
| | | TAGTCTCACCAGTCTTAGCATGTTCCAGAT |
| | | ACCAGAACTTTGAGTAATTACAGTATGGTA |
| | | CCCCCATTAGATCTCTTAGATGATTCCTCA |
| | | TCAACAGCTGATCGGAAATCAGAGAATTTA |
| | | CTGTTGTTTTGAATACATGCAAGGCAGACT |
| | | CTACATCTTGCTTGAACTTACTCAGGGCGG |
| | | CCTTGTTGTAATCAATTAGTCGTAGCATGT |
| | | CACAGAACTCTTCATCATGATTGACATTAC |
| | | ATTTTGCAACAGCTGTATTCCCAAAACATT |
| | | TGAGCTCTGCAGCAAGGATCATCCATTTGG |
| | | TCAGGCAATAACCACCTGGATTTTCTACTC |
| | | CTGAGGAGTCTGACAGGGTCCAGGTGAATG |
| | | TGCCTGCAAGTCTCCTAGTGAGAAACTTTG |
| | | TCTTTTCCTGAGCAAAGAGGATTCTAGACA |
| | | TCCCAAAAGGGCCTGCATATCTACAGTGGT |
| | | TTTCCCAAGTCCTGTTTTGTATGATTAGGT |
| | | ACTGATAGCTTGTTTGGCTGCACCAAGTGG |
| | | TCTTGCCATCTGAACCTGCCCAGCCCCAGC |
| | | CACTTCTCATGTATTTTCCTCCAAAGGCAG |
| | | TTCTAAACATGTCCAAGACTCTACCTCTGA |
| | | AAGTCCTACACTGGCTTATAGCGCTCTGTG |
| | | GGTCCGAAAATGACAAGTTGTATTGAATGG |
| | | TGATGCCATTGTTAAAATCACAAGACACTG |
| | | CTTTGTGGTTGGAATTCCCTCTAATACTGA |
| | | GGTGCAGACTCGAGACTATACTCATGAGTG |
| | | TATGGTCAAAAGTCTTTTTGTTGAAAGCGG |
| | | AGGTTAAGTTGCAAAAATTGTGATTAAGGA |
| | | TGGAGTCGTTAGTGAAAGTTAGCTCCAGTC |
| | | CAGAGCTTCCCATACTGATGTAGTGATGAG |
| | | AGTTGTTGGCTGAGCACGCATTGGGCATCG |
| | | TCAGATTTAAGTGAGACATATCAAACTCCA |
| | | CTGATTTGAACTGGTAAACCCCTTTATAGA |
| | | TGTCGGGACCATTAAGGCCGTACATGCCAC |
| | | AGGACCTACCAGCCAAAAAAAGGAAGCTGA |
| | | CCAGTGCTAATATCCCACAGGTGGCGAAAT |
| | | TGTACACAGCTTTGATGCTCGTGATTATAA |
| | | TGAGCACAATAATGACAATGTTGATGACCT |
| | | CATCAATGATGTGAGGCAAAGCCTCAAACA |
| | | TTGTCACAATCTGACCCATCTTGTTGCTCA |
| | | ATGGTTTCTCAAGACAAATGCGCAATCAAA |
| | | TGCCTAGGATCCACTGTGCG |
| 29 | Nucleotide | GCGCACCGGGGATCCTAGGCATACCTTGGA |
| | sequence of | CGCGCATATTACTTGATCAAAGATGTACAA |
| | artPICV- | GCTGGTGGTAGTTGGAGCAGTGGGAGTGGG |
| | 5xKRASmut-H1- | CAAATCAGCCTTGACCATTGGAGGCTCAGG |
| | NP-S-segment | TGGTGGAGGCTCAGGAGGCTACAAACTTGT |
| | | GGTGGTTGGAGCCAGAGGTGTAGGCAAAAG |
| | | TGCACTCACCATAGGAGGAAGTGGAGGCGG |
| | | AGGTAGTGGAGGCTACAAACTTGTTGTTGT |
| | | TGGGGCCTGTGGGGTGGGAAAGTCAGCACT |
| | | CACCATTGGTGGCAGTGGAGGTGGAGGCAG |
| | | TGGTGGCTACAAGTTGGTGGTCGTAGGTGC |
| | | TGGGGATGTGGGCAAATCTGCCCTCACAAT |
| | | TGGAGGCTCAGGAGGTGGAGGCAGTGGTGG |
| | | CTACAAATTGGTTGTGGTGGGTGCAGATGG |
| | | TGTGGGCAAAAGTGCTTTGACCATCTGAGC |
| | | CCTAGCCTCGACATGGGCCTCGACGTCACT |
| | | CCCCAATAGGGGAGTGACGTCGAGGCCTCT |
| | | GAGGACTTGAGCTCAGAGGTTGATCAGATC |
| | | TGTGTTGTTCCTGTACAGCGTGTCAATAGG |
| | | CAAGCATCTCATCGGCTTCTGGTCCCTAAC |
| | | CCAGCCTGTCACTGTTGCATCAAACATGAT |
| | | GGTATCAAGCAATGCACAGTGAGGATTCGC |
| | | AGTGGTTTGTGCAGCCCCCTTCTTCTTCTT |
| | | CTTTATGACCAAACCTTTATGTTTGGTGCA |
| | | GAGTAGATTGTATCTCTCCCAGATCTCATC |
| | | CTCAAAGGTGCGTGCTTGCTCGGCACTGAG |
| | | TTTCACGTCAAGCACTTTTAAGTCTCTTCT |
| | | CCCATGCATTTCGAACAAACTGATTATATC |
| | | ATCTGAACCTTGAGCAGTGAAAACCATGTT |
| | | TTGAGGTAAATGTCTGATGATTGAGGAAAT |
| | | CAGGCCTGGTTGGGCATCAGCCAAGTCCTT |
| | | TAAAAGGAGACCATGTGAGTACTTGCTTTG |
| | | CTCTTTGAAGGACTTCTCATCGTGGGGAAA |
| | | TCTGTAACAATGTATGTAGTTGCCCGTGTC |
| | | AGGCTGGTAGATGGCCATTTCCACCGGATC |
| | | ATTTGGTGTTCCTTCAATGTCAATCCATGT |
| | | GGTAGCTTTTGAATCAAGCATCTGAATTGA |
| | | GGACACAACAGTATCTTCTTTCTCCTTAGG |
| | | GATTTGTTTAAGGTCCGGTGATCCTCCGTT |
| | | TCTTACTGGTGGCTGGATAGCACTCGGCTT |
| | | CGAATCTAAATCTACAGTGGTGTTATCCCA |
| | | AGCCCTCCCTTGAACTTGAGACCTTGAGCC |
| | | AATGTAAGGCCAACCATCCCCTGAAAGACA |
| | | AATCTTGTATAGTAAATTTTCATAAGGATT |
| | | TCTCTGTCCGGGTGTAGTGCTCACAAACAT |
| | | ACCTTCACGATTCTTTATTTGCAATAGACT |
| | | CTTTATGAGAGTACTAAACATAGAAGGCTT |
| | | CACCTGGATGGTCTCAAGCATATTGCCACC |
| | | ATCAATCATGCAAGCAGCTGCTTTGACTGC |
| | | TGCAGACAAACTGAGATTGTACCCTGAGAT |
| | | GTTTATGGCTGATGGCTCATTACTAATGAT |
| | | TTTTAGGGCACTGTGTTGCTGTGTGAGTTT |
| | | CTCTAGATCTGTCATGTTCGGGAACTTGAC |
| | | AGTGTAGAGCAAACCAAGTGCACTCAGCGC |
| | | TTGGACAACATCATTAAGTTGTTCACCCCC |
| | | TTGCTCAGTCATACAAGCGATGGTTAAGGC |
| | | TGGCATTGATCCAAATTGATTGATCAACAA |
| | | TGTATTATCCTTGATGTCCCAGATCTTCAC |
| | | AACCCCATCTCTGTTGCCTGTGGGTCTAGC |
| | | ATTAGCGAACCCCATTGAGCGAAGGATTTC |
| | | GGCTCTTTGTTCCAACTGAGTGTTTGTGAG |
| | | ATTGCCCCCATAAACACCAGGCTGAGACAA |
| | | ACTCTCAGTTCTAGTGACTTTCTTTCTTAA |
| | | CTTGTCCAAATCAGATGCAAGCTCCATTAG |
| | | CTCCTCTTTGGCTAAGCCTCCCACCTTAAG |
| | | CACATTGTCCCTCTGGATTGATCTCATATT |
| | | CATCAGAGCATCAACCTCTTTGTTCATGTC |
| | | TCTTAACTTGGTCAGATCAGAATCAGTCCT |
| | | TTTATCTTTGCGCATCATTCTTTGAACTTG |
| | | AGCAACTTTGTGAAAGTCAAGAGCAGATAA |
| | | CAGTGCTCTTGTGTCCGACAACACATCAGC |
| | | CTTCACAGGATGGGTCCAGTTGGATAGACC |
| | | CCTCCTAAGGGACTGTACCCAGCGGAATGA |
| | | TGGGATGTTGTCAGACATTTTGGGGTTGTT |
| | | TGCACTTCCTCCGAGTCAGTGAAGAAGTGA |
| | | ACGTACAGCGTGATCTAGAATCGCCTAGGA |
| | | TCCACTGTGCG |
| 30 | Nucleotide | GCGCACCGGGGATCCTAGGCATACCTTGGA |
| | sequence | CGCGCATATTACTTGATCAAAGATGTACAA |
| | artPCIV- | GCTGGTGGTAGTTGGAGCAGTGGGAGTGGG |
| | 5xKRASmut-H1- | CAAATCAGCCTTGACCATTGGAGGCTCAGG |
| | GP-S-segment | TGGTGGAGGCTCAGGAGGCTACAAACTTGT |
| | | GGTGGTTGGAGCCAGAGGTGTAGGCAAAAG |
| | | TGCACTCACCATAGGAGGAAGTGGAGGCGG |
| | | AGGTAGTGGAGGCTACAAACTTGTTGTTGT |
| | | TGGGGCCTGTGGGGTGGGAAAGTCAGCACT |
| | | CACCATTGGTGGCAGTGGAGGTGGAGGCAG |
| | | TGGTGGCTACAAGTTGGTGGTCGTAGGTGC |
| | | TGGGGATGTGGGCAAATCTGCCCTCACAAT |
| | | TGGAGGCTCAGGAGGTGGAGGCAGTGGTGG |
| | | CTACAAATTGGTTGTGGTGGGTGCAGATGG |
| | | TGTGGGCAAAAGTGCTTTGACCATCTGAGC |
| | | CCTAGCCTCGACATGGGCCTCGACGTCACT |
| | | CCCCAATAGGGGAGTGACGTCGAGGCCTCT |
| | | GAGGACTTGAGCTTATTTACCCAGTCTCAC |
| | | CCATTTGTAGGGTTTCTTTGGGATTTTATA |
| | | ATACCCACAGCTGCAAAGAGAGTTCCTAGT |
| | | AATCCTATGTGGCTTCGGACAGCCATCACC |
| | | AATGATGTGCCTATGAGTGGGTATTCCAAC |
| | | TAAGTGGAGAAACACTGTGATGGTGTAAAA |
| | | CACCAAAGACCAGAAGCAAATGTCTGTCAA |
| | | TGCTAGTGGAGTCTTACCTTGTCTTTCTTC |
| | | ATATTCTTTTATCAGCATTTCATTGTACAG |
| | | ATTCTGGCTCTCCCACAACCAATCATTCTT |
| | | AAAATGCGTTTCATTGAGGTACGAGCCATT |
| | | GTGAACTAACCAACACTGCGGTAAAGAATG |
| | | TCTCCCTGTGATGGTATCATTGATGTACCA |
| | | AAATTTTGTATAGTTGCAATAAGGGATTTT |
| | | GGCAAGCTGTTTGAGACTGTTTCTAATCAC |
| | | AAGTGAGTCAGAAATAAGTCCGTTGATAGT |
| | | CTTTTTAAAGAGATTCAACGAATTCTCAAC |
| | | ATTAAGTTGTAAGGTTTTGATAGCATTCTG |
| | | ATTGAAATCAAATAACCTCATCGTATCGCA |
| | | AAATTCTTCATTGTGATCTTTGTTGCATTT |
| | | TGCCATCACAGTGTTATCAAAACATTTTAT |
| | | TCCAGCCCAAACAATAGCCCATTGCTCCAA |
| | | ACAGTAACCACCTGGGACATGTTGCCCAGT |
| | | AGAGTCACTCAAGTCCCAAGTGAAAAAGCC |
| | | AAGGAGTTTCCTGCTCACAGAACTATAAGC |
| | | AGTTTTTTGGAGAGCCATCCTTATTGTTGC |
| | | CATTGGAGTATATGTACAGTGATTTTCCCA |
| | | TGTGGTGTTCTGTATGATCAGGAAATTGTA |
| | | ATGTGTCCCACCTTCACAGTTTGTTAGTCT |
| | | GCAAGACCCTCCACTACAGTTATTGAAACA |
| | | TTTTCCAACCCACGCAATTTTTGGGTCCCC |
| | | AATGATTTGAGCAAGCGACGCAATAAGATG |
| | | TCTGCCAACCTCACCTCCTCTATCCCCAAC |
| | | TGTCAAGTTGTACTGGATCAACACCCCAGC |
| | | ACCCTCAACTGTTTTGCATCTGGCACCTAC |
| | | ATGACGAGTGACATGGAGCACATTGAAGTG |
| | | TAACTCATTAAGCAACCATTTTAATGTGTG |
| | | ACCTGCTTCTTCTGTCTTATCACAATTACT |
| | | AATGTTACCATATGCAAGGCTTCTGATGTT |
| | | GGAAAAGTTTCCAGTAGTTTCATTTGCAAT |
| | | GGATGTGTTTGTCAAAGTGAGTTCAATTCC |
| | | CCATGTTGTGTTAGATGGTCCTTTGTAGTA |
| | | ATGATGTGTGTTGTTCTTGCTACATGATTG |
| | | TGGCAAGTIGTCAAACATTCTTGTGAGGTT |
| | | GAACTCAACGTGGGTGAGATTGTGCCTCCT |
| | | ATCAATCATCATGCCATCACAACTTCTGCC |
| | | AGCCAAAATGAGGAAGGTGATGAGTTGGAA |
| | | TAGGCCACATCTCATCAGATTGACAAATCC |
| | | TTTGATGATGCATAGGGTTGAGACAATGAT |
| | | TAAGGCGACATTGAACACCTCCTGCAGGAC |
| | | TTCGGGTATAGACTGGATCAAAGTCACAAC |
| | | TTGTCCCATTTTGGGGTTGTTTGCACTTCC |
| | | TCCGAGTCAGTGAAGAAGTGAACGTACAGC |
| | | GTGATCTAGAATCGCCTAGGATCCACTGTG |
| | | CG |
| 31 | Amino acid | YKLVVVGAVGVGKSALTI- |
| | suence of | YKLVVVGAGDVGKSALTI- |
| | 4xKRASmut | YKLVVVGADGVGKSALTI- |
| | (18-mer) | YKLVVVGACGVGKSALTI |
| 32 | Nucleotide | ATGTACAAGCTGGTGGTGGTGGGAGCTGTG |
| | sequence of | GGAGTGGGAAAGTCTGCCCTCACCATCTAC |
| | 4xKRASmut | AAGCTGGTGGTAGTTGGAGCTGGAGATGTT |
| | (18-mer) | GGCAAATCAGCTCTCACCATCTACAAACTG |
| | | GTTGTAGTGGGGGCAGATGGGGTGGGCAAA |
| | | AGTGCCCTCACCATTTACAAGCTGGTGGTG |
| | | GTTGGAGCCTGTGGAGTGGGCAAAAGTGCC |
| | | CTCACCATCTAA |
| 33 | Nucleotide | ATGTACAAGCTGGTGGTGGTGGGAGCTGGA |
| | sequence | GGAGTGGGCAAGAGTGCCCTGACCATCTAA |
| | KRASwt | |

### 8. EXAMPLES

### 8.1 Design of Arenavirus Vector

(a) artLCMV-4xKRASmut

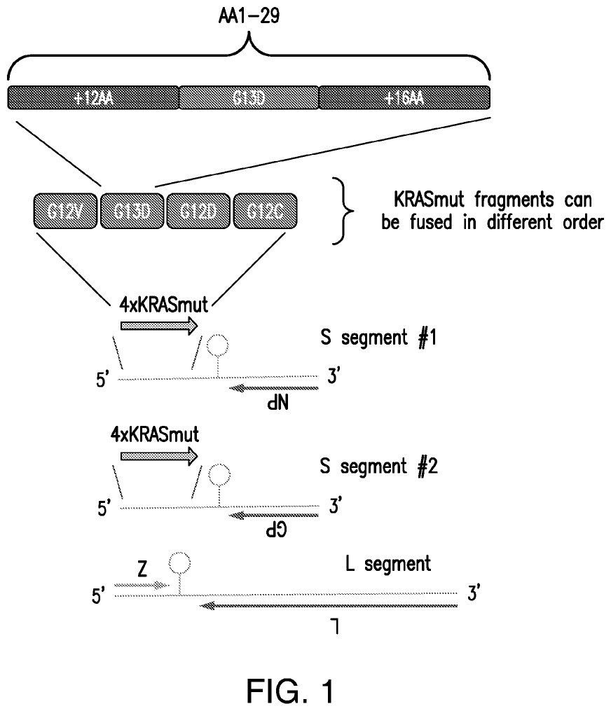

artLCMV-4xKRASmut is an attenuated, replication competent, tri-segmented vector based on LCMV clone 13 (LCMV cl13) expressing the GP of LCMV strain WE instead of its endogenous glycoprotein (LCMV cl13/WE) (FIG. 1). The NP-S segment includes a nucleotide sequence encoding four copies of KRAS amino acids 1-29 with G121D, G12V, G12C and G13D mutations, respectively (SEQ ID NO: 1) and the GP-S segment includes a nucleotide sequence encoding four copies of KRAS amino acids 1-29 with G121D, G12V, G12C and G13D mutations, respectively (SEQ TD NO: 1). The nucleotide sequences of KRAS epitopes are modified to be devoid of CpG dinucleotide motifs. The vector is generated de novo by electroporation of production cells using a five-plasmid co-transfection system, as described previously by Kallert et al. Nat Commun 2017; 8:15327.

(b) artLCMV-4xKRASmut_E7

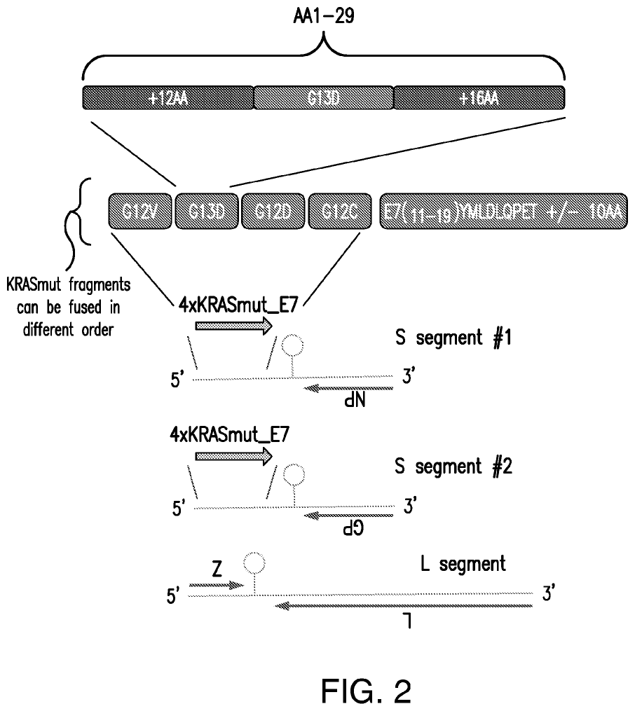

artLCMV-4xKRASmut_E7 is an attenuated, replication competent, tri-segmented vector based on LCMV clone 13 (LCMV c113) expressing the GP of LCMV strain WE instead of its endogenous glycoprotein (LCMV cl13/WE) (FIG. 2). The NP-S segment includes a nucleotide sequence encoding four copies of KRAS amino acids 1-29 with G12D, G12V, G12C and G13D mutations, respectively, and an HPV E7 epitope (SEQ ID NO:2) and the GP-S segment includes a nucleotide sequence encoding four copies of KRAS amino acids 1-29 with G12D, G12V, G12C and G13D mutations, respectively, and an HPV E7 epitope (SEQ ID NO:2). The nucleotide sequences of the KRAS epitopes and the HPV E7 epitope are modified to be devoid of CpG dinucleotide motifs. The vector is generated de novo by electroporation of production cells using a five-plasmid co-transfection system, as described previously by Kallert et al. Nat Commun 2017; 8:15327.

(c) artLCMV-4xKRASmut_EBV

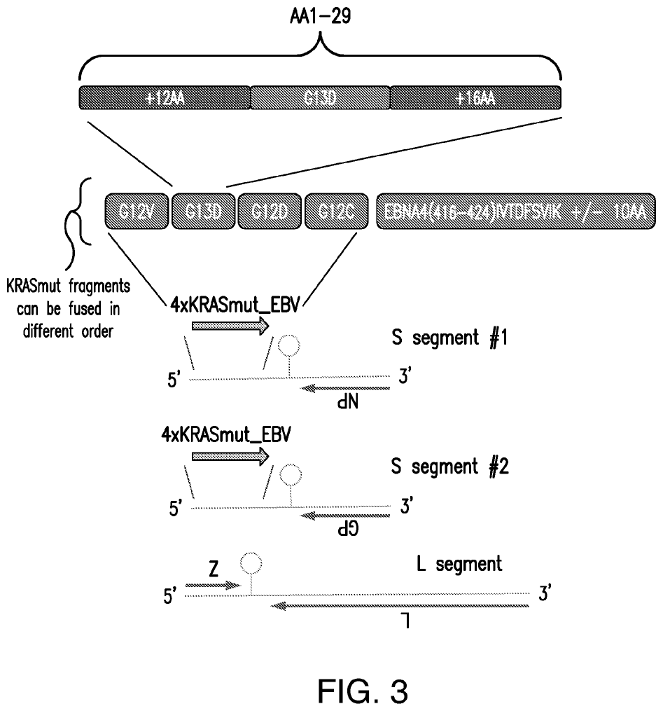

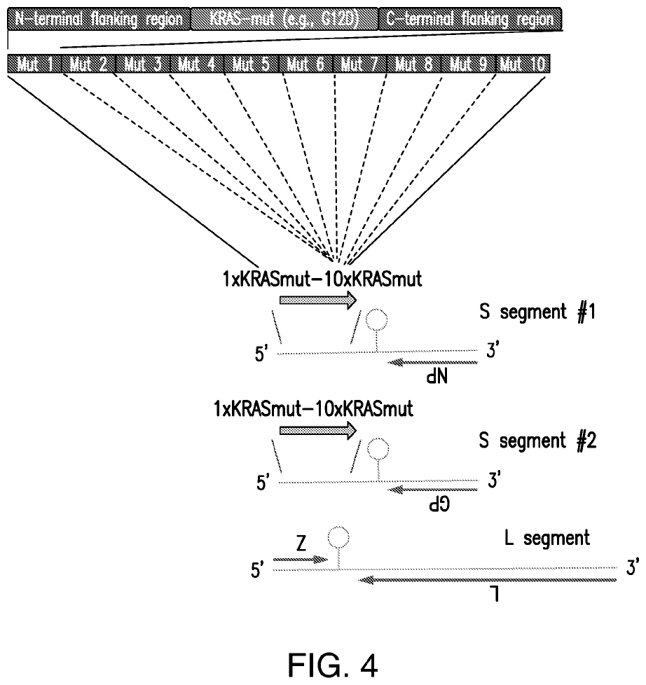

artLCMV-4xKRASmut_EBV is an attenuated, replication competent, tri-segmented vector based on LCMV clone 13 (LCMV c113) expressing the GP of LCMV strain WE instead of its endogenous glycoprotein (LCMV c113/WE) (FIG. 3). The NP-S segment includes a nucleotide sequence encoding four copies of KRAS amino acids 1-29 with G12D, G12V, G12C and G13D mutations, respectively, and an EBV epitope (SEQ ID NO:3) and the GP-S segment includes a nucleotide sequence encoding four copies of KRAS amino acids 1-29 with G12D, G12V, G12C and G13D mutations, respectively, and an EBV epitope (SEQ ID NO:3). The nucleotide sequences of the KRAS epitopes and the HPV E7 epitope are modified to be devoid of CpG dinucleotide motifs. The vector is generated de novo by electroporation of production cells using a five-plasmid co-transfection system, as described previously by Kallert et al. Nat Commun 2017; 8:15327.

(d) artPICV-4xKRASmut

artPICV-4xKRASmut is an attenuated, replication competent, tri-segmented vector based on virulent strain passage 18 of Pichinde Virus (PIC; alternatively named PICV p18) (FIG. 1). The NP-S segment includes a nucleotide sequence encoding four copies of KRAS amino acids 1-29 with G12D, G12V, G12C and G13D mutations, respectively (SEQ ID NO:1) and the GP-S segment includes a nucleotide sequence encoding four copies of KRAS amino acids 1-29 with G12D, G12V, G12C and G13D mutations, respectively (SEQ ID NO:1). The nucleotide sequences of KRAS epitopes are modified to be devoid of CpG dinucleotide motifs. The vector is generated de novo by electroporation of production cells using a five-plasmid co-transfection system, as described previously by Kallert et al. Nat Commun 2017; 8:15327.

(e) artPICV-4xKRASmut_E7

artPICV-4xKRASmut_E7 is an attenuated, replication competent, tri-segmented vector based on virulent strain passage 18 of Pichinde Virus (PIC; alternatively named PICV p18) (FIG. 2). The NP-S segment includes a nucleotide sequence encoding four copies of KRAS amino acids 1-29 with G12D, G12V, G12C and G13D mutations, respectively, and an HPV E7 epitope (SEQ ID NO:2) and the GP-S segment includes a nucleotide sequence encoding four copies of KRAS amino acids 1-29 with G12D, G12V, G12C and G13D mutations, respectively, and an HPV E7 epitope (SEQ ID NO:2). The nucleotide sequences of the KRAS epitopes and the HPV E7 epitope are modified to be devoid of CpG dinucleotide motifs. The vector is generated de novo by electroporation of production cells using a five-plasmid co-transfection system, as described previously by Kallert et al. Nat Commun 2017; 8:15327.

(f) artPICV-4xKRASmut_EBV

artPICV-4xKRASmut_EBV is an attenuated, replication competent, tri-segmented vector based on virulent strain passage 18 of Pichinde Virus (PIC; alternatively named PICV p18) (FIG. 3). The NP-S segment includes a nucleotide sequence encoding four copies of KRAS amino acids 1-29 with G12D, G12V, G12C and G13D mutations, respectively, and an EBV epitope (SEQ ID NO:3) and the GP-S segment includes a nucleotide sequence encoding four copies of KRAS amino acids 1-29 with G12D, G12V, G12C and G13D mutations, respectively, and an EBV epitope (SEQ ID NO:3). The nucleotide sequences of the KRAS epitopes and the HPV E7 epitope are modified to be devoid of CpG dinucleotide motifs. The vector is generated de novo by electroporation of production cells using a five-plasmid co-transfection system, as described previously by Kallert et al. Nat Commun 2017; 8:15327.

(g) artLCMV-14xp53mut

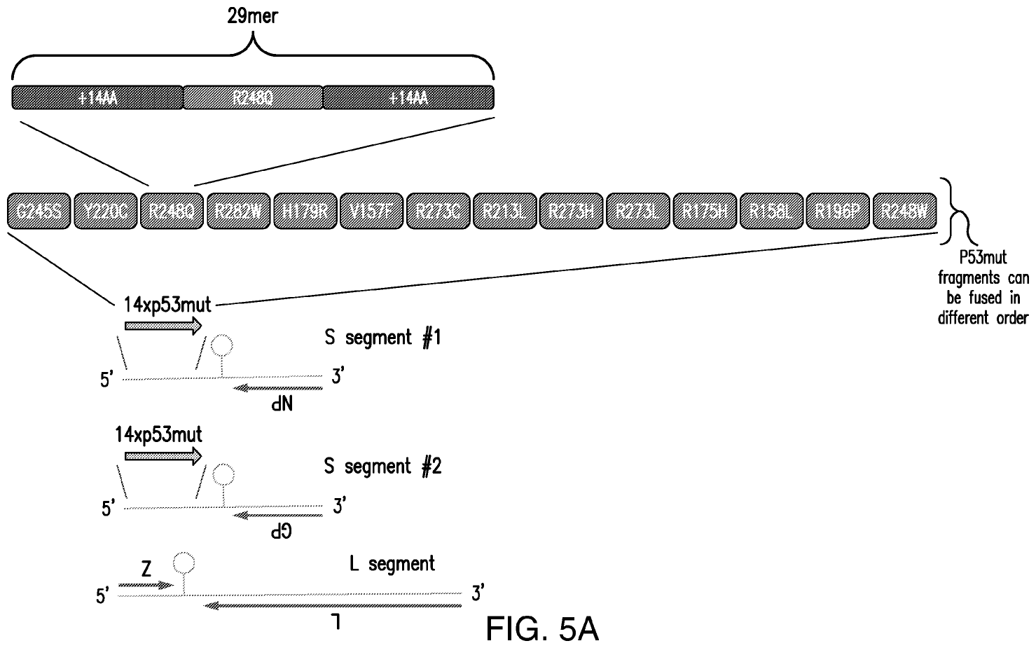

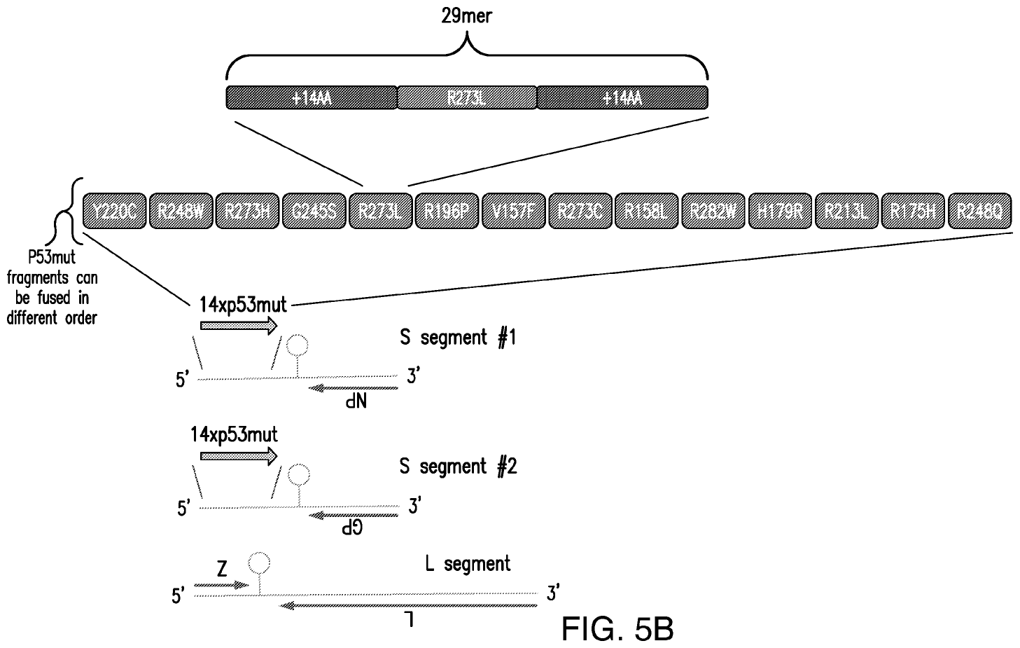

artLCMV-14xp53mut is an attenuated, replication competent, tri-segmented vector based on LCMV clone 13 (LCMV c113) expressing the GP of LCMV strain WE instead of its endogenous glycoprotein (LCMV c113/WE) (FIG. 5A). The NP-S segment includes a nucleotide sequence encoding 14 p53 neoepitopes, each consisting of 29 amino acids (SEQ ID NO:7) and the GP-S segment includes a nucleotide sequence encoding 14 p53 neoepitopes, each consisting of 29 amino acids (SEQ ID NO:7). The nucleotide sequences encoding the p53 neoepitopes are modified to be devoid of CpG dinucleotide motifs. The vector is generated de novo by electroporation of production cells using a five-plasmid co-transfection system, as described previously by Kallert et al. Nat Commun 2017; 8:15327.

(h) artLCMV-14xp53mut_E7

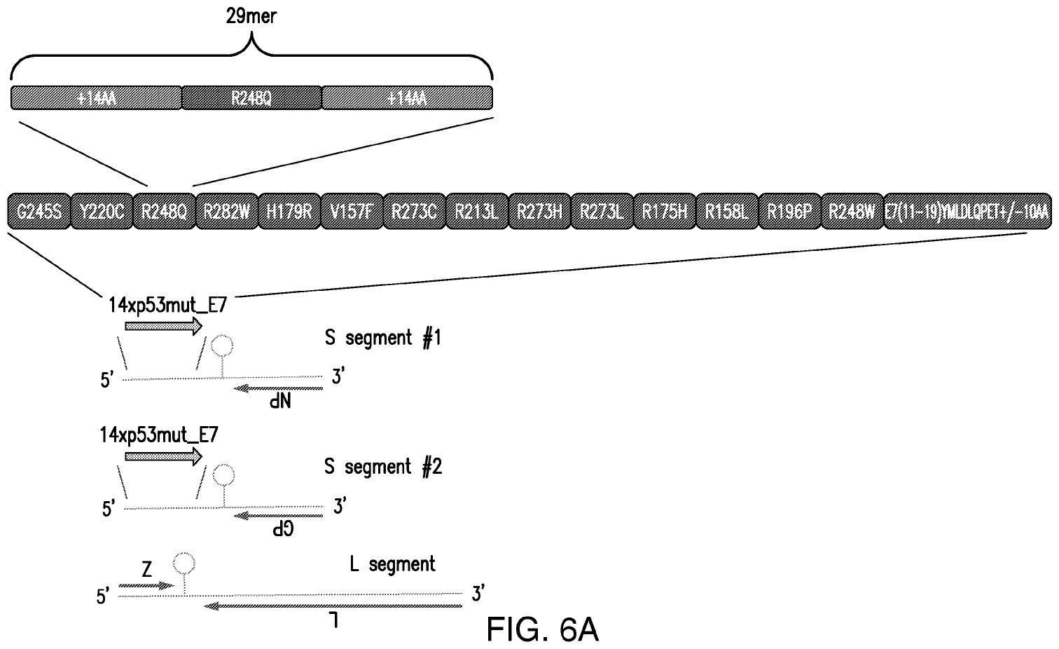

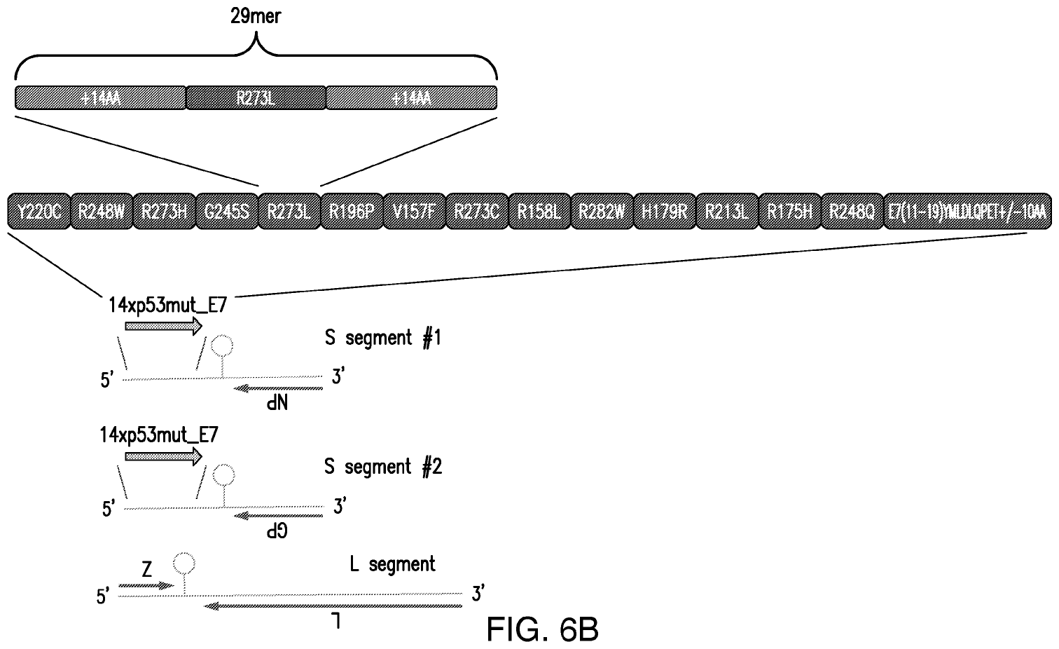

artLCMV-14xp53mut_E7 is an attenuated, replication competent, tri-segmented vector based on LCMV clone 13 (LCMV c113) expressing the GP of LCMV strain WE instead of its endogenous glycoprotein (LCMV c113/WE) (FIG. 6A). The NP-S segment includes a nucleotide sequence encoding 14 p53 neoepitopes, each consisting of 29 amino acids, and an HPV E7 epitope (SEQ ID NO:8) and the GP-S segment includes a nucleotide sequence encoding 14 p53 neoepitopes, each consisting of 29 amino acids, and an HPV E7 epitope (SEQ ID NO:8). The nucleotide sequences encoding the p53 neoepitopes and the HPV E7 epitope are modified to be devoid of CpG dinucleotide motifs. The vector is generated de novo by electroporation of production cells using a five-plasmid co-transfection system, as described previously by Kallert et al. Nat Commun 2017; 8:15327.

(i) artLCMV-14xp53mut_EBV

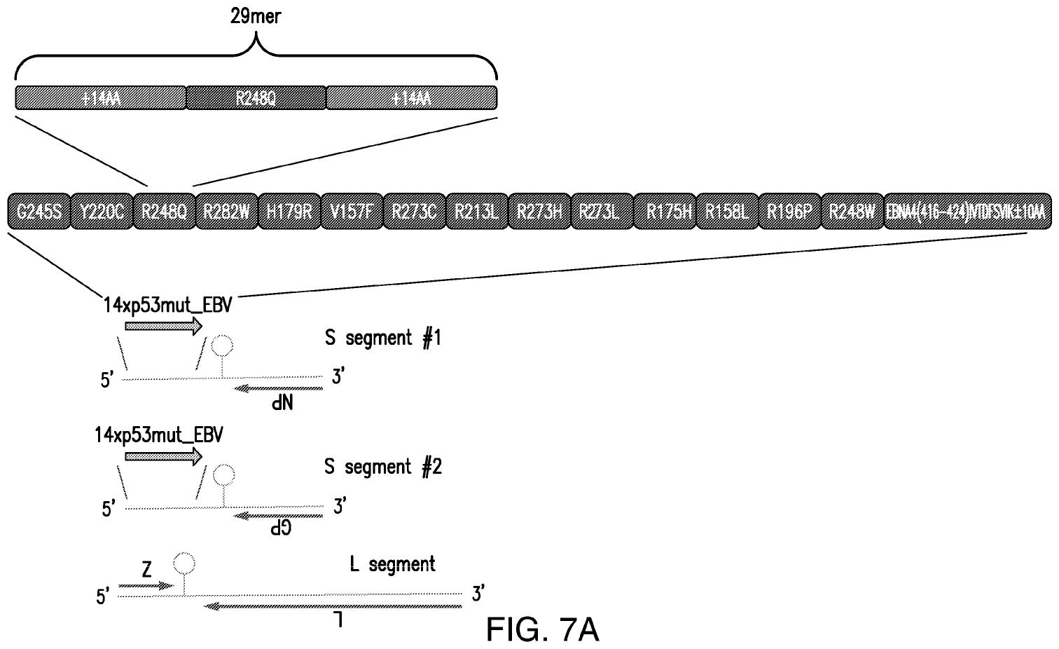

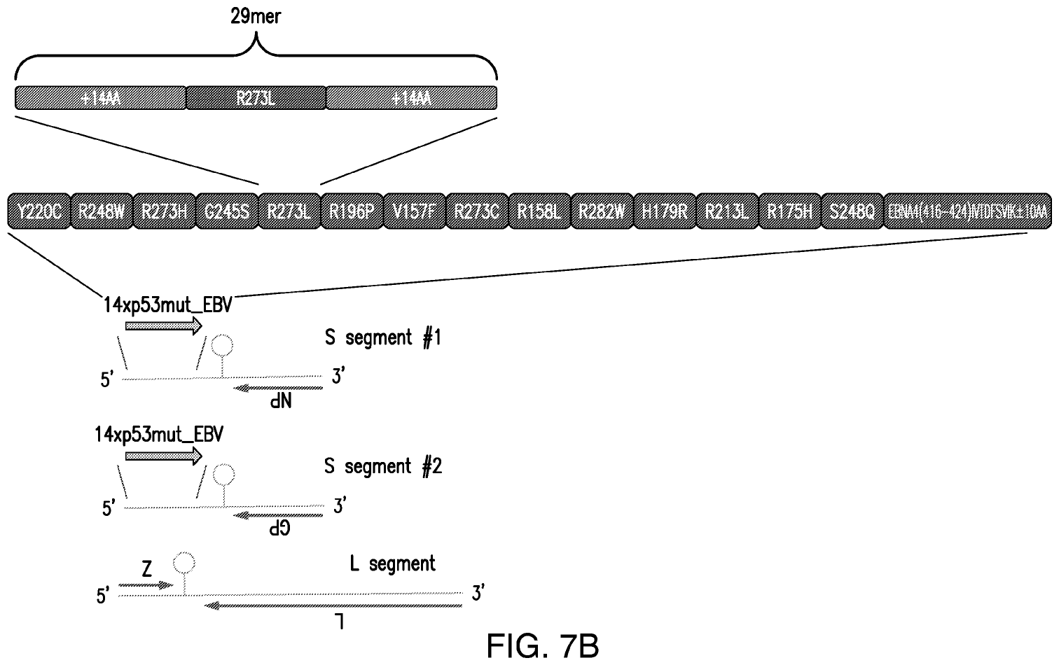

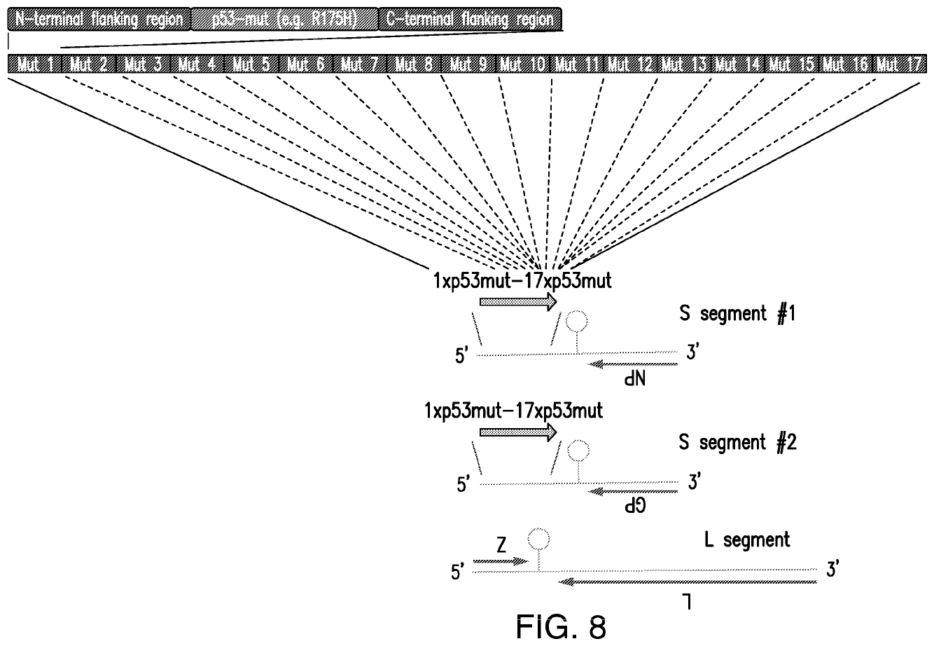

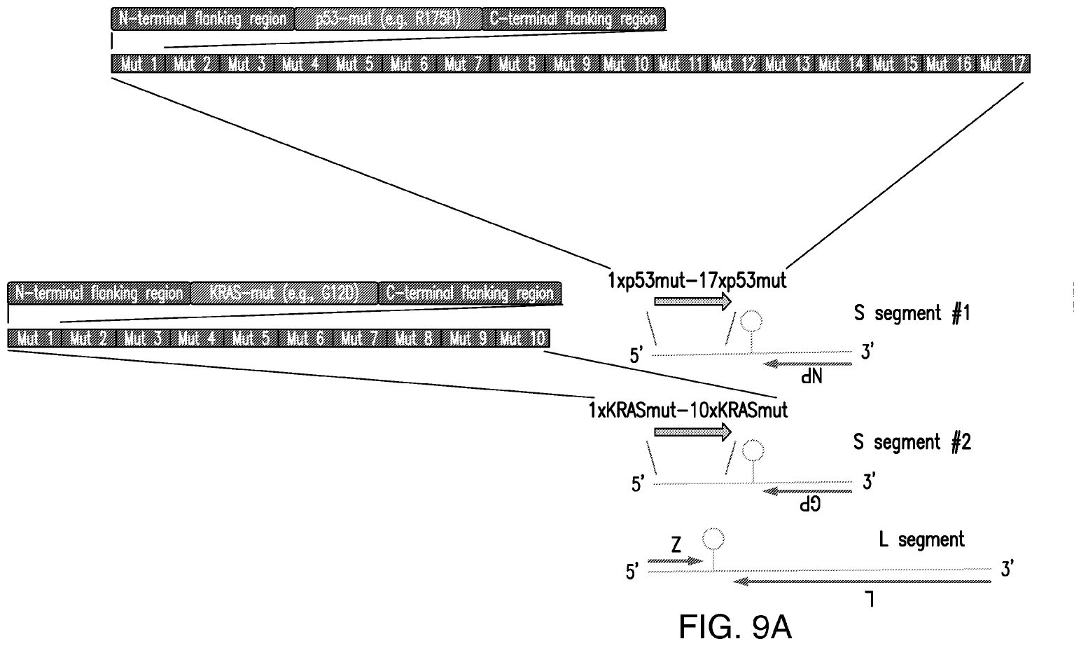

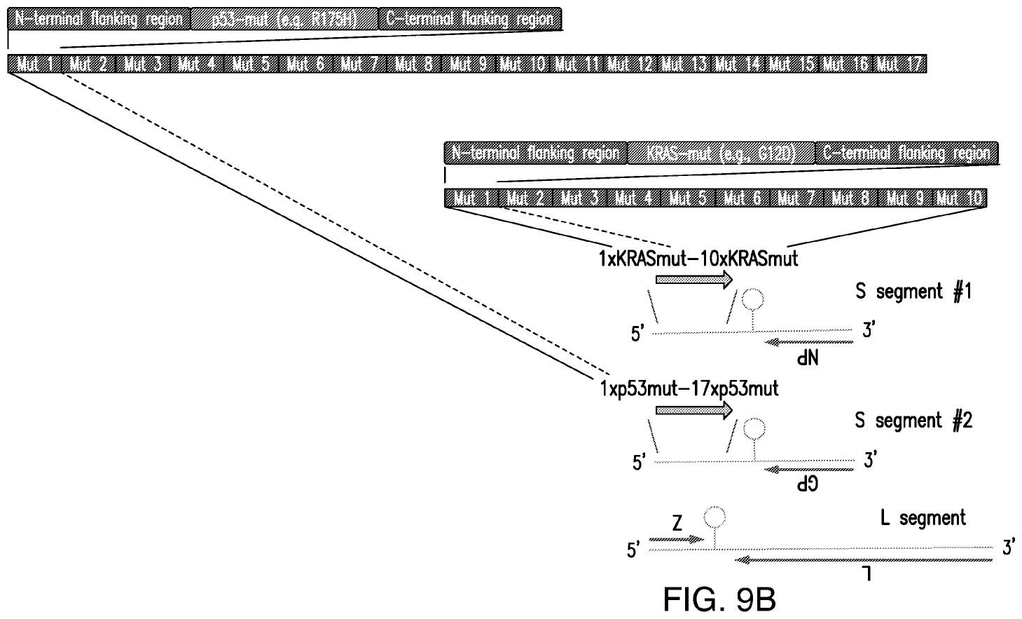

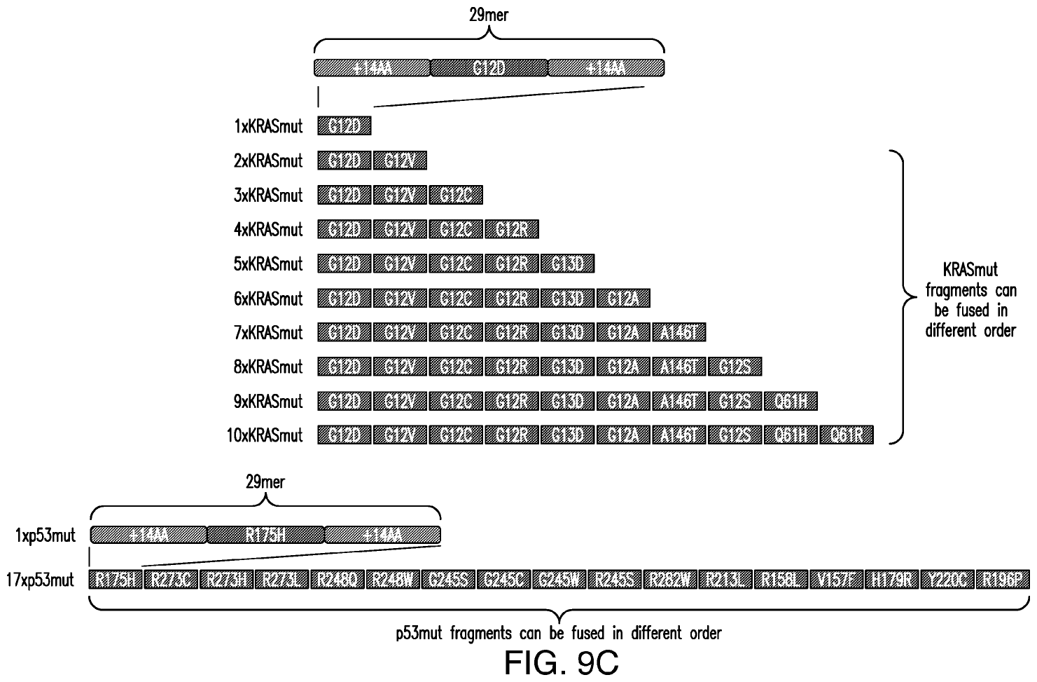

artLCMV-14xp53mut_EBV is an attenuated, replication competent, tri-segmented vector based on LCMV clone 13 (LCMV c113) expressing the GP of LCMV strain WE instead of its endogenous glycoprotein (LCMV c113/WE) (FIG. 7A). The NP-S segment includes a nucleotide sequence encoding 14 p53 neoepitopes, each consisting of 29 amino acids, and an EBV epitope (SEQ ID NO:9) and the GP-S segment includes a nucleotide sequence encoding 14 p53 neoepitopes, each consisting of 29 amino acids, and an EBV epitope (SEQ ID NO:9). The nucleotide sequences encoding the p53 neoepitopes are modified to be devoid of CpG dinucleotide motifs. The vector is generated de novo by electroporation of production cells using a five-plasmid co-transfection system, as described previously by Kallert et al. Nat Commun 2017; 8:15327.

(j) artPICV-14xp53mut

artPICV-14xp53mut is an attenuated, replication competent, tri-segmented vector based on virulent strain passage 18 of Pichinde Virus (PIC; alternatively named PICV p18) (FIG. 5B). The NP-S segment includes a nucleotide sequence encoding 14 p53 neoepitopes, each consisting of 29 amino acids (SEQ ID NO:10) and the GP-S segment includes a nucleotide sequence encoding 14 p53 neoepitopes, each consisting of 29 amino acids (SEQ ID NO:10). The nucleotide sequences encoding the p53 neoepitopes are modified to be devoid of CpG dinucleotide motifs. The vector is generated de novo by electroporation of production cells using a five-plasmid co-transfection system, as described previously by Kallert et al. Nat Commun 2017; 8:15327.

(k) artPICV-14xp53mut_E7

artPICV-14xp53mut_E7 is an attenuated, replication competent, tri-segmented vector based on virulent strain passage 18 of Pichinde Virus (PIC; alternatively named PICV p18) (FIG. 6B). The NP-S segment includes a nucleotide sequence encoding 14 p53 neoepitopes, each consisting of 29 amino acids, and an HPV E7 epitope (SEQ ID NO:11) and the GP-S segment includes a nucleotide sequence encoding 14 p53 neoepitopes, each consisting of 29 amino acids, and an HPV E7 epitope (SEQ ID NO:11). The nucleotide sequences encoding the p53 neoepitopes and the HPV E7 epitope are modified to be devoid of CpG dinucleotide motifs. The vector is generated de novo by electroporation of production cells using a five-plasmid co-transfection system, as described previously by Kallert et al. Nat Commun 2017; 8:15327.

(l) artPICV-14xp53mut_EBV

artPICV-14xp53mut_EBV is an attenuated, replication competent, tri-segmented vector based on virulent strain passage 18 of Pichinde Virus (PIC; alternatively named PICV p18) (FIG. 7B). The NP-S segment includes a nucleotide sequence encoding 14 p53 neoepitopes, each consisting of 29 amino acids, and an EBV epitope (SEQ ID NO:12) and the GP-S segment includes a nucleotide sequence encoding 14 p53 neoepitopes, each consisting of 29 amino acids, and an EBV epitope (SEQ ID NO:12). The nucleotide sequences encoding the p53 neoepitopes are modified to be devoid of CpG dinucleotide motifs. The vector is generated de novo by electroporation of production cells using a five-plasmid co-transfection system, as described previously by Kallert et al. Nat Commun 2017; 8:15327.

(m) artLCMV-5xKRASmut-H2

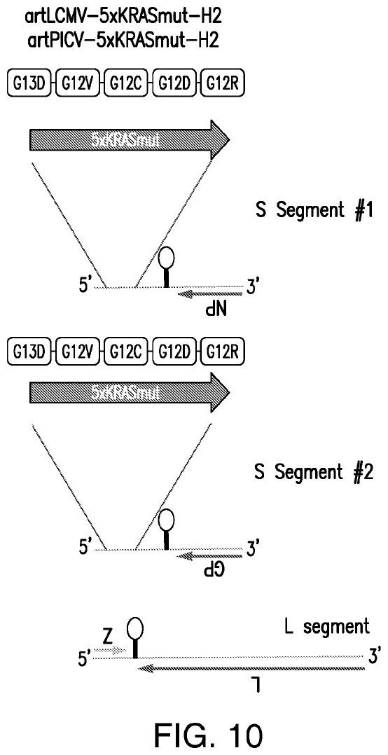

artLCMV-5xKRASmut-H2 is an attenuated, replication competent, tri-segmented vector based on LCMV clone 13 (LCMV c113) expressing the GP of LCMV strain WE instead of its endogenous glycoprotein (LCMV c113/WE). The NP-S segment as well as the GP-S segment encode for an antigenic insert comprised of five mutant epitopes of KRAS, each consisting of 18 amino acids (SEQ ID NO:19). The nucleotide sequence of the antigenic insert was modified to be devoid of CpG dinucleotide motifs. The vector was generated de novo by electroporation of production cells using a five-plasmid co-transfection system, as described previously by Kallert et al. Nat Commun 2017; 8:15327. FIG. 10 shows a schematic representation of artLCMV-5xKRASmut-H2.

(n) artPICV-5xKRASmut-H2

artPICV-5xKRASmut-H2 is an attenuated, replication competent, tri-segmented vector based on virulent strain passage 18 of Pichinde Virus (PIC; alternatively named PICV p18). The NP-S segment as well as the GP-S segment encode for an antigenic insert comprised of five mutant epitopes of KRAS, each consisting of 18 amino acids (SEQ ID NO:19). The nucleotide sequence of the antigenic insert was modified to be devoid of CpG dinucleotide motifs. The vector was generated de novo by electroporation of production cells using a five-plasmid co-transfection system, as described previously by Kallert et al. Nat Commun 2017; 8:15327. FIG. 10 shows a schematic representation of artPICV-5xKRASmut-H2.

### 8.2 In Vitro T Cell Activation and Function

### (a) Human PBMC Activation Assay

Human PBMCs are isolated from healthy, HLA-A2+ donors and pulsed with HLA-A2-restricted KRASmut and control peptides. T cell activation is measured by ELISPOT, proliferation (FACS CFSE) and cytokine expression profile (supernatant, intracellular FACS staining).

### (b) In Vitro T Cell Activation System

Monocyte-derived dendritic cells (moDCs) or artificial antigen presenting cells (aAPCs) are infected with arenavirus particles encoding antigenic fragment(s) of mutant KRAS (KRASmut vectors) and control vectors (i.e., arenavirus particles that do not encode an antigenic fragment of mutant KRAS) and incubated with isolated human T cells. T cell activation is measured by ELISPOT, proliferation (FACS CFSE) and cytokine expression profile (supernatant, intracellular FACS staining).

### (c) Cytotoxicity

In vitro killing of KRASmut human cancer cell lines (i.e., human cancer cell lines that express mutant KRAS) by human T cells primed with KRASmut vectors is assessed by LDH release, ⁵¹Cr release, flow cytometry or live cell imaging assays.

### (d) KRAS-Reporter Cell Assay

KRAS-mut specific T cell receptor (TCR) activation is measured with reporter cell lines which have been genetically engineered to express KRAS-mut specific TCRs and produce a bioluminescent or fluorescent signal induced by TCR signaling. These cells are incubated with HLA-matched, KRASmut vector transfected cell lines. 8.3 In vivo Immunogenicity and Efficacy

### (a) Immunogenicity

Human HLA-transgenic mice are immunized with KRASmut vectors and control vectors. PBMCs and/or splenocytes of immunized animals are isolated and KRASmut-specific T cell responses are measured by ELISPOT, intracellular cytokine staining, MHC multimer staining and by multiplex cytokine profiling.

Humanized mice are immunized with KRASmut vectors and control vectors. PBMCs and/or splenocytes of immunized animals are isolated and KRASmut-specific T cell responses are measured by ELISPOT, intracellular cytokine staining, MHC multimer staining and by multiplex cytokine profiling.

To analyze the ability of vector constructs encoding different combinations of mutated KRAS epitopes (i.e., fragments of mutant KRAS) to induce an antigen-specific immune response in mice transgenic for HLA-A*11 (i.e., CB6F1-Tg(HLA-A*1101/H2-Kb)A11.01 mice), intravenous (i.v.) immunization was performed with the indicated vector constructs at 1×10⁵ RCV FFU/dose.

Table 2, below, is an illustration of the experiment to test in vivo immunogenicity of vectors described in the present application.

TABLE 2

| | Prime Boost Vaccination | | | | | | | |
|---|---|---|---|---|---|---|---|---|
| | | | | | | | | No. of |
| | | | Dose | | | | | HLA- |
| | | | (RCV | Day | Day | | | A*11:01 |
| Group | Vector Prime | Vector Boost | FFU) | Prime | Boost | Route | ELISPOT | mice |
| 1 | Formulation | Formulation | N/A | Day 0 | Day 21 | i.v. | Spleen | 5 |
| | Buffer | Buffer | | | | | Day 26 | |
| 2 | artPICV- | artLCMV- | 1 × 10⁵ | | | | | 5 |
| | 5xKRASmut-H2 | 5xKRASmut-H2 | | | | | | |
| 3 | artPICV- | artLCMV- | | | | | | 5 |
| | 5xKRASmut-H1 | 5xKRASmut-H1 | | | | | | |
| 4 | artPICV- | artLCMV- | | | | | | 5 |
| | 4xKRASmut(18-mer) | 4xKRASmut(18-mer) | | | | | | |
| 5 | artPICV-KRASwt | artLCMV-KRASwt | | | | | | 5 |

On Day 0, mice transgenic for HLA-A*11 (i.e., CB6F1-Tg(HLA-A*1101/H2-Kb)A11.01 mice) were immunized intravenously with 1×10⁵ RCV FFU/dose of artPICV-5xKRASmut-H2 (group 2), artPICV-5xKRASmut-H1 (group 3), artPICV-4xKRASmut(18-mer) (group 4), or an artPICV-based vector encoding the corresponding 18-mer wild-type epitope of KRAS (i.e., artPICV-KRASwt) (group 5). Control mice were treated with formulation buffer only (group 1). On Day 21, mice were immunized intravenously with 1×10⁵ RCV FFU/dose of artLCMV-5xKRASmut-H2 (group 2), artLCMV-5xKRASmut-H1 (group 3), artLCMV-4xKRASmut(18-mer) (group 4), artLCMV-KRASwt (group 5) or were treated with formulation buffer (group 1). On Day 26, KRAS epitope-specific CD8+ T cell responses were analyzed by ELISPOT analysis using wild-type and mutation-specific KRAS-based peptides for stimulation. A mixture of NP-based peptides derived from LCMV and PICV was used as control.

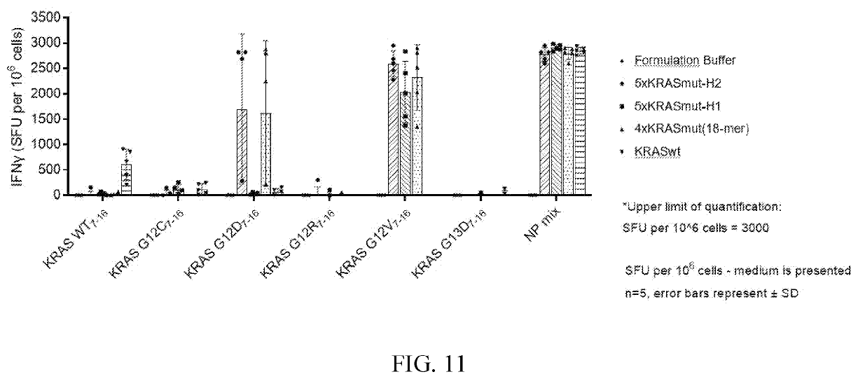

As shown in FIG. 11, CD8+ T cell responses directed against two of the encoded mutated KRAS epitopes (i.e., KRAS G12D and KRAS G12V) could be detected in animals of group 2 (treated with the combination of artPICV- and artLCMV-based vectors encoding the 5xKRASmut-H2 epitope cassette) as well as in mice of group 4 (treated with the combination of artPICV- and artLCMV-based vectors encoding the 4xKRASmut(18-mer) epitope cassette). Surprisingly, in contrast, in animals of group 3 (treated with the combination of artPICV- and artLCMV-based vectors encoding the 5xKRASmut-H1 epitope cassette), CD8+ T cell responses could only be observed against one of the encoded mutated KRAS epitopes (i.e., KRAS G12D), whereas this vector combination did not induce detectable immune responses against the KRAS G12V epitope. Importantly, none of the tested vector constructs encoding mutated epitopes of KRAS induced a detectable CD8+ T cell response against the wild-type KRAS protein.

### (b) Efficacy

### (i) CT26 Model

Balb/c mice with subcutaneously transplanted CT26 (KRAS-mut) tumors are injected with KRASmut vectors, control vectors, or buffer. Tumor control is assessed by monitoring tumor growth (caliper measurement) after vector treatment. KRASmut-specific T cell response is analyzed by MHC multimer staining and flow cytometry.

### (ii) KPC PDAC Model

KPC mice harboring mutations in KRAS (e.g., G12D) and Tp53 (e.g., R172H) spontaneously develop tumors and build metastases in lung and liver. KPC mice or mice transplanted with KPC tumors are injected with KRASmut vectors, control vectors or buffer. Primary tumor and metastasis control is assessed by histological analysis of pancreas, liver and lymph node after vector treatment. KRASmut-specific T cell response is analyzed by MHC multimer staining and flow cytometry.

(iii) Humanized PDX and KRAS-mut tumor cell lines

Humanized mice are transplanted with patient-derived HLA-matched human KRASmut xenografts or human KRASmut tumor cell lines and injected with KRASmut vectors, control vectors or buffer. Tumor control is assessed by monitoring tumor growth (caliper measurement) after vector treatment. KRASmut-specific T cell response is analyzed by MHC multimer staining and flow cytometry.

### 8.4 Transgene Stability

(i) artLCMV-5xKRASmut-H2

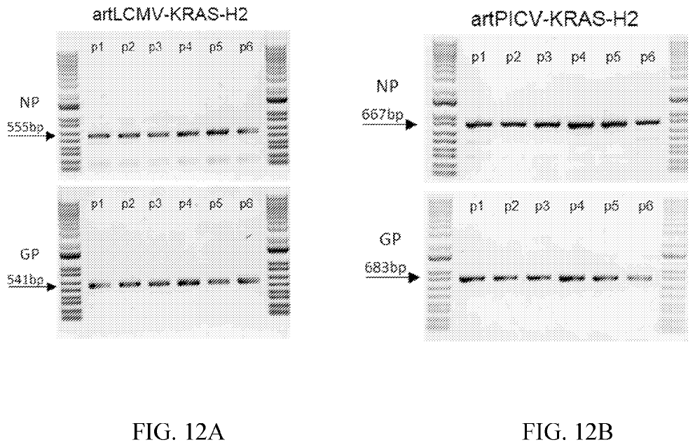

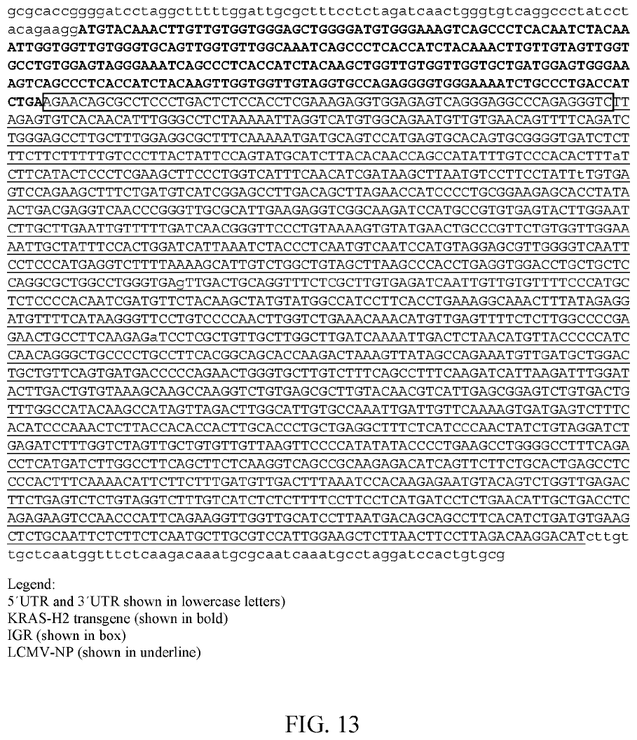

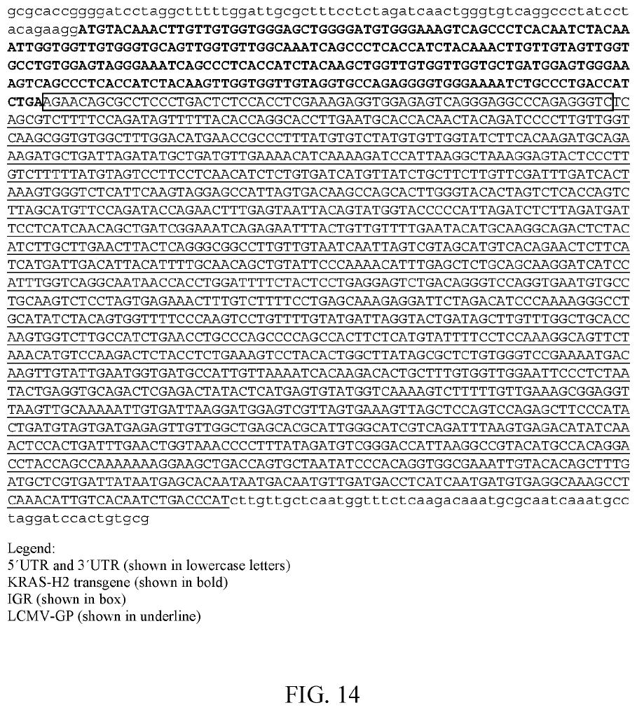

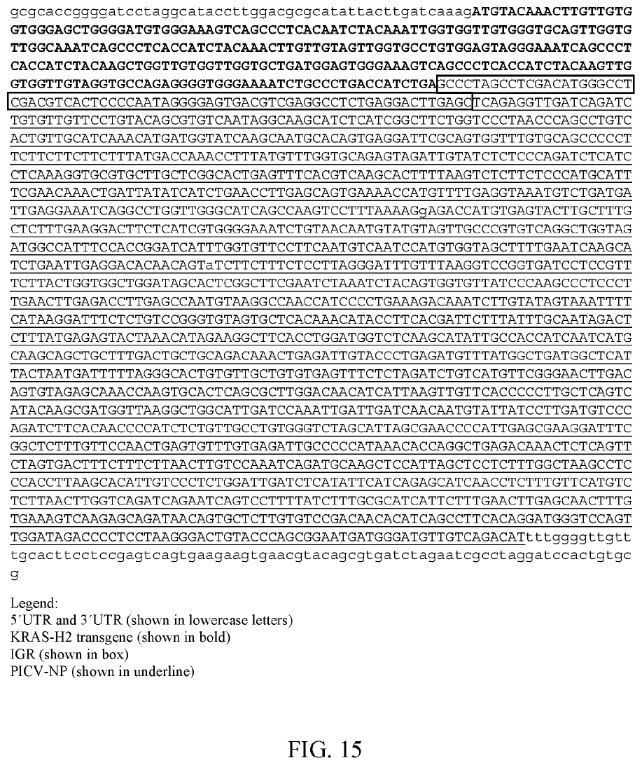

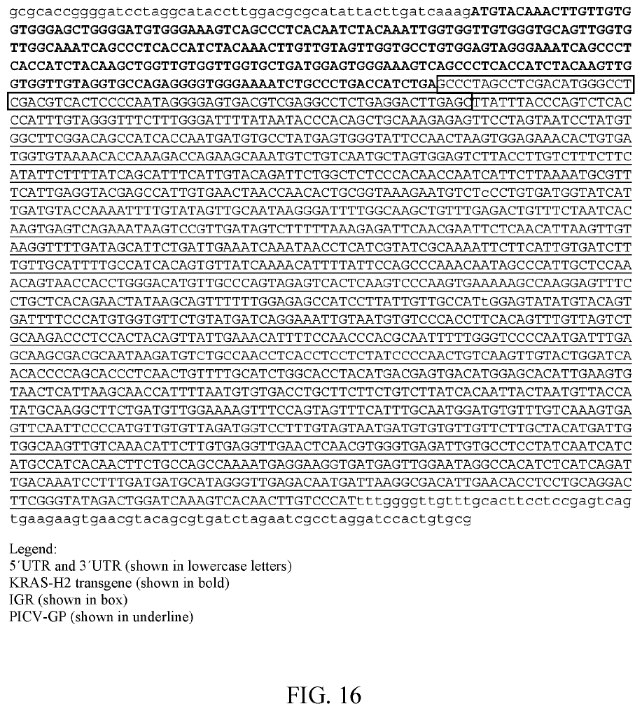

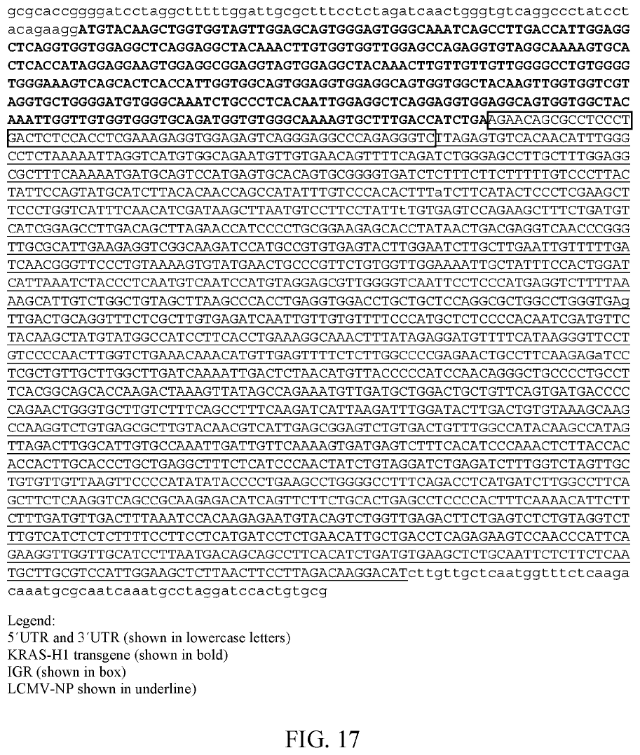

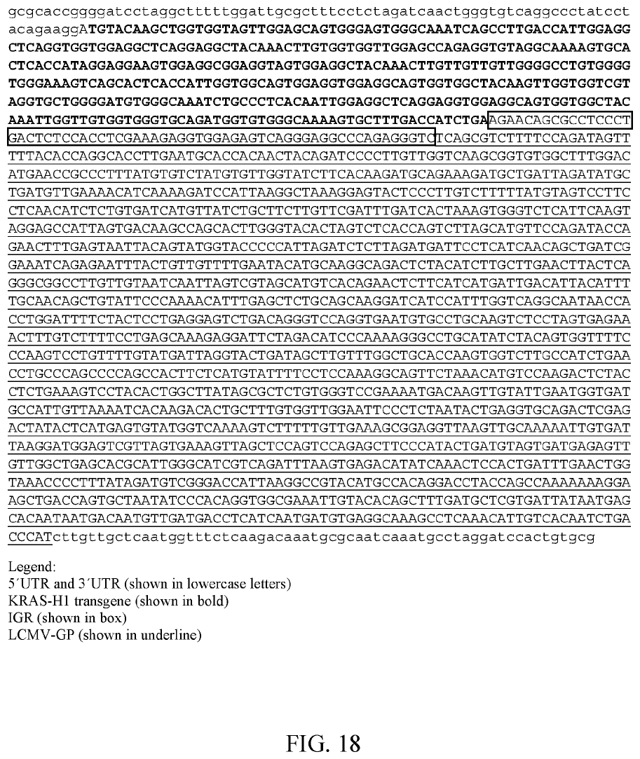

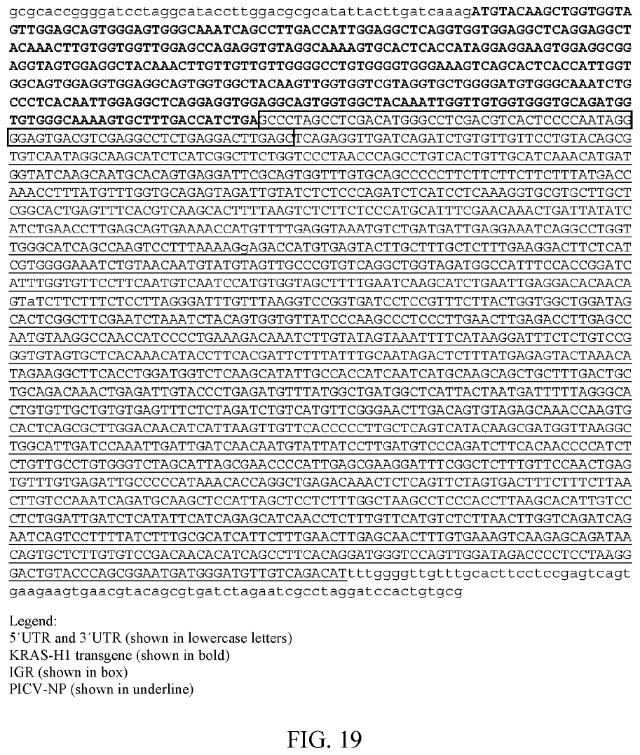

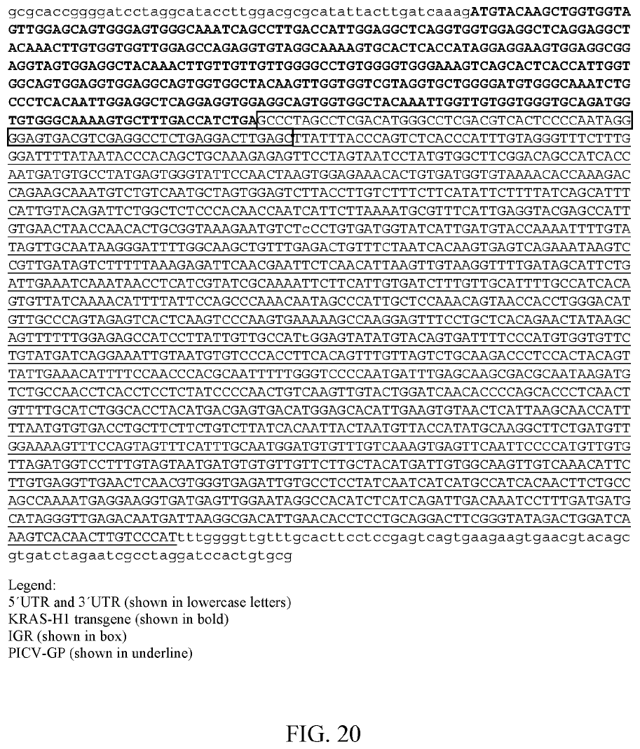

The genetic stability of the encoded transgene after generation of the artLCMV-5xKRASmut-H2 vector was analyzed by PCR at increasing passage levels (FIG. 12A). The 5xKRASmut transgene was stable among all tested passage levels.

(ii) artPICV-5xKRASmut-H2

The genetic stability of the encoded transgene after generation of the artPICV-5xKRASmut-H2 vector was analyzed by PCR at increasing passage levels (FIG. 12B). The 5xKRASmut transgene was stable among all tested passage levels.

### 8.5 Vector Immunogenicity

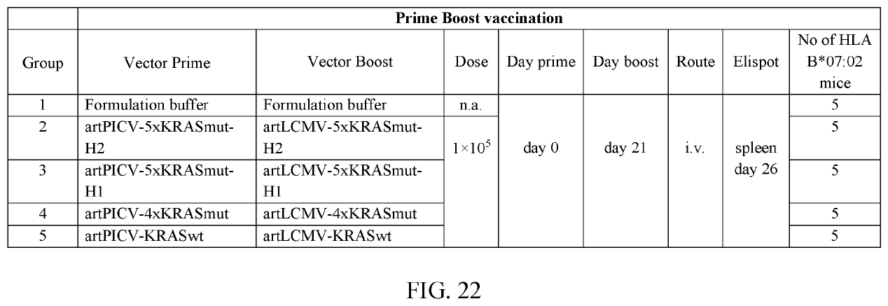

To analyze the ability of vector constructs encoding different combinations of mutated KRAS epitopes to induce an antigen-specific immune response, intravenous immunization was performed in mice with the indicated vector constructs at 1×10⁵ RCV FFU/dose (see FIG. 22 for the study design).

On Day 0 mice transgenicforlHLA-B*07 (i.e., CB6F1-Tg(HLA-B*0702/H2-Kb)B7.xx mice) were immunized intravenously with 1×10⁵ RCV FFU/dose of artPICV-5xKRASmut-H2 (group 2), artPICV-5xKRASmut-H1 (group 3), artPICV-4xKRASmut (group 4), or an artPICV-based vector encoding the corresponding 18-mer wild-type epitope of KRAS (i.e., artPICV-KRASwt) (group 5). Control mice were treated with formulation buffer only (group 1). Twenty-one days later, mice were immunized intravenously with 1×10⁵ RCV FFU/dose of artLCMV-5xKRASmut-H2 (group 2), artLCMV-5xKRASmut-H1 (group 3), artLCMV-4xKRASmut (group 4), artLCMV-KRASwt (group 5), or were treated with formulation buffer (group 1). KRAS epitope-specific CD8 T cell responses were analyzed on day 26 by ELISpot analysis using wild-type and mutation-specific KRAS-based peptides for stimulation. A mixture of NP-based peptides derived from LCMV and PICV was used as control.

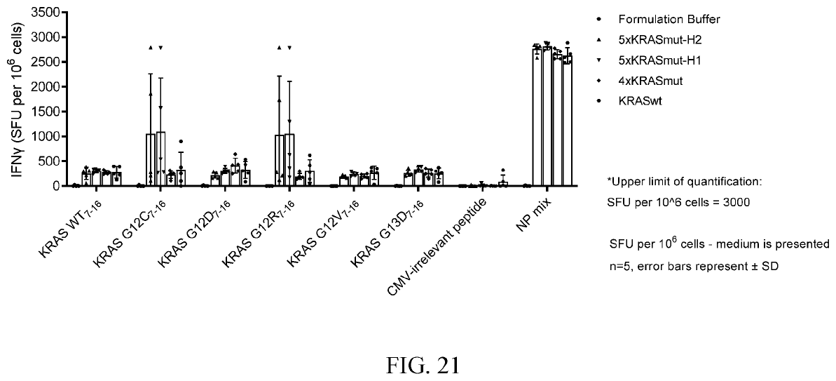

As shown in FIG. 21, CD8 T cell responses directed against two of the encoded mutated KRAS epitopes (i.e., KRAS G12C and KRAS G12R) could be detected in (2 out of 5) the animals of group 2 (treated with the combination of artPICV- and artLCMV-based vectors encoding the 5xKRASmut-H2 epitope cassette) as well as in (2 out of 5) mice of group 3 (treated with the combination of artPICV- and artLCMV-based vectors encoding the 5xKRASmut-H1 epitope cassette). In contrast, in animals of group 4 (treated with the combination of artPICV- and artLCMV-based vectors encoding the 4xKRASmut epitope cassette), CD8 T cell responses could not be observed. Moreover, none of the tested vector constructs encoding mutated epitopes of KRAS induced a detectable CD8 T cell response against the wild-type KRAS protein.

### 8.6 Vector Immunogenicity

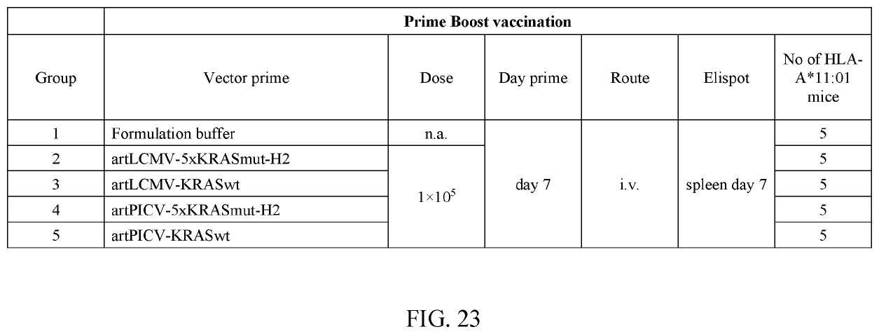

To analyze the ability of vector constructs encoding mutated KRAS epitopes to induce an antigen-specific immune response, intravenous immunization was performed in mice with the indicated vector constructs at 1×10⁵ RCV FFU/dose (see FIG. 23 for the study design).

On Day 0, mice transgenic for HLA-A*11 (i.e., CB6F1-Tg(HLA-A*1101/H2-Kb)A11.01 mice) were immunized intravenously with 1×10⁵ RCV FFU/dose of artLCMV-5xKRASmut-H2 (group 2), artPICV-5xKRASmut-H2 (group 4), or an artLCMV and artPICV-based vector encoding the corresponding 18-mer wild-type epitope of KRAS (i.e., artLCMV-KRASwt and artPICV-KRASwt) (groups 3 and 5). Control mice were treated with formulation buffer only (group 1). KRAS epitope-specific CD8 T cell responses were analyzed on day 7 post immunization by ELISpot analysis using wild-type and mutation-specific KRAS-based peptides for stimulation. A mixture of NP-based peptides derived from LCMV was used as control. NP-based peptides derived from PICV were not detected, due to a technical error.

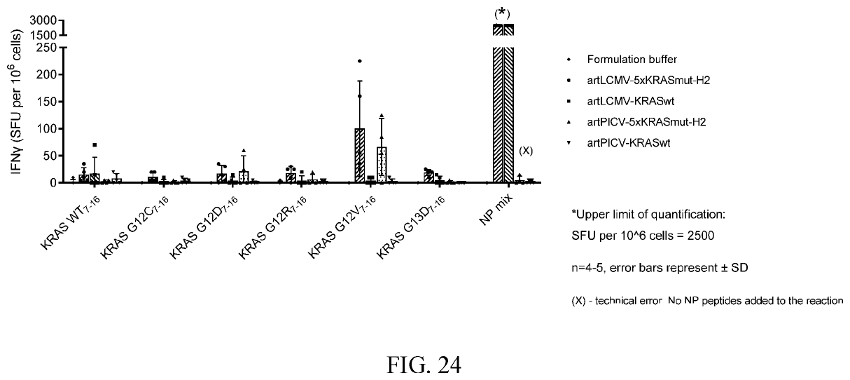

As shown in FIG. 24, CD8 T cell responses directed against KRAS G12V could be detected in animals of group 2 (treated with artLCMV-5xKRASmut-H2) as well in mice of group 4 (treated with artLCMV-5xKRASmut-H2). Moreover, none of the tested vector constructs encoding mutated epitopes of KRAS induced a detectable CD8 T cell response against the wild-type KRAS protein.

What is claimed is:

1. An arenavirus particle, wherein
a. the arenavirus particle comprises an arenavirus genome comprising a nucleotide sequence encoding an antigenic fragment of mutant KRAS, wherein the antigenic fragment comprises the mutation in KRAS; and
b. at least one arenavirus open reading frame (“ORF”) of the arenavirus genome is either (i) functionally inactivated or deleted; or (ii) located in a position other than the wild-type position of said at least one arenavirus ORF; or (iii) sequestered into two or more functional fragments and a fragment of the at least one arenavirus ORF is located in a position other than the wild-type position of said at least one arenavirus ORF.

2. The arenavirus particle of claim 1, wherein the mutation in KRAS is at amino acid position G12, G13, A18, A59, Q61, K117, A146, or D119 of KRAS.

3. The arenavirus particle of any of the preceding claims, wherein the mutation in KRAS is A18D, A59E, A59G, A59P, A59T, A59S, A59V, A146P, A146S, A146T, A146V, D119N, G12A, G12C, G12D, G12F, G12L, G12R, G12S, G12V, G13A, G13C, G13D, G13E, G13R, G13S, G13V, K117N, Q61E, Q61H, Q61K, Q61L, Q61P, Q61R or a combination thereof.

4. The arenavirus particle of any of the preceding claims, wherein the mutation in KRAS is G12A, G12C, G12D, G12R, G12S, G12V, G13D, Q61H, Q61R, A146T or a combination thereof.

5. The arenavirus particle of claim 1, wherein the mutation in KRAS is one, more, or all of G13D, G12V, G12C, G12D, and G12R.

6. The arenavirus particle of any one of claims 1-5, wherein the nucleotide sequence encodes from N- to C-terminus fragments of mutant KRAS comprising the mutations G13D, G12V, G12C, G12D, and G12R, respectively.

7. The arenavirus particle of claim 5, wherein the mutations in KRAS is all of G13D, G12V, G12C, G12D, and G12R in any possible order.

8. The arenavirus particle of claim 1, wherein the arenavirus genome comprises a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of SEQ ID NO:20.

9. The arenavirus particle of claim 1, wherein the arenavirus genome comprises a nucleotide sequence encoding an expression product whose amino acid sequence is at least 80%, 81%, 82%, 83%, 84%, 85%, 86%, 87%, 88%, 89%, 90%, 91%9, 92%, 93%, 94%, 95%, 96%, 97%, 98%, 99%, or 100% identical to the amino acid sequence of SEQ ID NO:19.

10. The arenavirus particle of any of the preceding claims, wherein the fragment of mutant KRAS is 8, 9, 10, 11, 12, 13, 14, 15, 16, 17, 18, 19, 20, 21, 22, 23, 24, 25, 26, 27, 28, 29, or 30 amino acids long.

11. The arenavirus particle of any of the preceding claims, wherein the fragment of mutant KRAS is 18 amino acids long.

12. The arenavirus particle of any of the preceding claims, wherein the region flanking the mutation at the N-terminus of the antigenic fragment is 0, 1, 2, 3, 4, 5, 6, 7, 8, 9, 10, 11, 12, 13, 14, 15, 16, 17, 18, 19, or 20 amino acids long.

13. The arenavirus particle of any of the preceding claims, wherein the region flanking the mutation at the N-terminus of the antigenic fragment is 8 or 9 amino acids long.

14. The arenavirus particle of any of the preceding claims, wherein the region flanking the mutation at the C-terminus of the antigenic fragment is 0, 1, 2, 3, 4, 5, 6, 7, 8, 9, 10, 11, 12, 13, 14, 15, 16, 17, 18, 19, or 20 amino acids long.

15. The arenavirus particle of any of the preceding claims, wherein the region flanking the mutation at the C-terminus of the antigenic fragment is 8 or 9 amino acids long.

16. The arenavirus particle of any of the preceding claims, wherein the nucleotide sequence encodes two, three, four, five, six, seven, eight, nine, 10, 11, 12, 13, 14, 15, 16, 17, 18, 19, or 20 antigenic fragments of mutant KRAS, and wherein each antigenic fragment comprises the mutation of one of the mutant KRAS proteins.

17. The arenavirus particle of any of the preceding claims, wherein the nucleotide sequence encodes five antigenic fragments of a mutant KRAS, and wherein each antigenic fragment comprises the mutation of one of the mutant KRAS proteins.

18. The arenavirus particle of claim 17, wherein the five antigenic fragments of a mutant KRAS comprise the mutations G13D, G12V, G12C, G12D, and G12R.

19. The arenavirus particle of any of claims 16 to 17, wherein the antigenic fragments comprise the same or different mutations of mutant KRAS proteins.

20. The arenavirus particle of any of claims 16 to 18, wherein the antigenic fragments are fused to each other via the same or different linkers.

21. The arenavirus particle of any of claims 16 to 18, wherein the antigenic fragments are fused directly to each other without intervening sequences.

22. The arenavirus particle of claim 20, wherein the linker is AAY linker (AAY), AAA linker (AAA), GS linker (GGSGGGGSGG) (SEQ ID NO:42), or variants of AAY, AAA, and GS linker sequences optimized via in silico prediction.

23. The arenavirus particle of any of the preceding claims, wherein the nucleotide sequence is engineered to reduce or remove any CpG and TpA islands.

24. The arenavirus particle of claim 23, wherein the removal of the CpG and TpA islands comprises three cycles:
(i) CpG is removed in a first cycle;
(ii) TpA is removed in a second cycle; and
(iii) CpG is removed in a third cycle to remove newly introduced CpG in the second cycle.

25. The arenavirus particle of any of the preceding claims, wherein the arenavirus genome comprises:
(i) a first S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral glycoprotein (“GP”) under control of an arenavirus genomic 3′ UTR; and
(iii) an L-Segment.

26. The arenavirus particle of any of claims 1 to 19, 21, and 23 to 25, wherein the arenavirus genome comprises:
(i) a first S-Segment comprising the nucleotide sequence of SEQ ID NO:20 under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence of SEQ ID NO:20 under control of an arenavirus genomic 5′ UTR and an ORF encoding the arenaviral glycoprotein (“GP”) under control of an arenavirus genomic 3′ UTR; and
(iii) an L-Segment.

27. The arenavirus particle of any of claims 1 to 19, 21, and 23 to 26, wherein the arenavirus genome comprises:
(i) a first S-Segment comprising the nucleotide sequence of SEQ ID NO:21; and
(ii) a second S-Segment comprising the nucleotide sequence of SEQ ID NO:22; and
(iii) an L-Segment.

28. The arenavirus particle of any of claims 1 to 19, 21, and 23 to 26, wherein the arenavirus genome comprises:
(i) a first S-Segment comprising the nucleotide sequence of SEQ ID NO:23; and
(ii) a second S-Segment comprising the nucleotide sequence of SEQ ID NO:24; and
(iii) an L-Segment.

29. The arenavirus particle of any of claims 1 to 24, wherein the arenavirus genome comprises:
(i) a first S-Segment comprising an ORF encoding the arenavirus GP1 and GP2 subunits fused to a heterologous signal peptide under control of an arenavirus genomic 5′ UTR and an ORF encoding a fusion of arenavirus GP signal peptide and a nucleotide sequence encoding the antigenic fragment(s) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(iii) an L-Segment.

30. The arenavirus particle of claims 25 and 29, wherein the nucleotide sequence encoding the antigenic fragment(s) on the first S-Segment is different from the nucleotide sequence encoding the antigenic fragment(s) on the second S-Segment.

31. The arenavirus particle of claims 25 and 29, wherein the nucleotide sequence encoding the antigenic fragment(s) on the first S-Segment is the same as the nucleotide sequence encoding the antigenic fragment(s) on the second S-Segment.

32. The arenavirus particle of claims 25 and 29, wherein the antigenic fragment(s) encoded on the first S-Segment is/are different from the antigenic fragment(s) encoded on the second S-Segment.

33. The arenavirus particle of claims 25 and 29, wherein the antigenic fragment(s) encoded on the first S-Segment is/are the same as the antigenic fragment(s) encoded on the second S-Segment.

34. The arenavirus particle of claim 33, wherein the antigenic fragments encoded on the first S-Segment are the same as the antigenic fragments encoded on the second S-Segment but are fused to each other in a different order from the order in which the antigenic fragments encoded on the second S-Segment are fused to each other.

35. The arenavirus particle of any of claims 1 to 26 and 29 to 34, wherein the arenavirus genome further comprises a nucleotide sequence encoding an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation.

36. The arenavirus particle of any of claims 1 to 26 and 29 to 35, wherein the arenavirus genome further comprises a nucleotide sequence encoding an antigenic fragment of mutant PI3KCA, wherein the antigenic fragment comprises the E545K, H1047R and/or E542K mutation.

37. The arenavirus particle of any of claims 1 to 26 and 29 to 36, wherein the arenavirus genome further comprises a nucleotide sequence encoding an antigenic fragment of mutant BRAF, wherein the antigenic fragment comprises the V600E mutation.

38. The arenavirus particle of any of claims 1 to 26 and 29 to 37, wherein the arenavirus genome further comprises a nucleotide sequence encoding an antigenic fragment of mutant U2AF1, wherein the antigenic fragment comprises the S34F mutation.

39. The arenavirus particle of any of claims 1 to 26 and 29 to 38, wherein the arenavirus genome further comprises a nucleotide sequence encoding an antigenic fragment of mutant TP53, wherein the antigenic fragment comprises the G245S, Y220C, R248Q, R282W, H179R, V157F, R273C, R213L, R273H, R273L, R175H, R158L, R196P, R248W and/or C277F mutation.

40. The arenavirus particle of any of claims 1 to 26 and 29 to 39, wherein the arenavirus genome further comprises a nucleotide sequence encoding an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family.

41. The arenavirus particle of any of the preceding claims, wherein the arenavirus particle is derived from lymphocytic choriomeningitis virus (LCMV) or Pichinde virus.

42. A pharmaceutical composition comprising the arenavirus particle of any of the preceding claims.

43. A set of one or more nucleic acids encoding the genome of the arenavirus particle of any of claims 1 to 41.

44. A host cell comprising the set of one or more nucleic acids of claim 43.

45. A method of making the arenavirus particle of any of claims 1 to 41, wherein the method comprises culturing the host cell of claim 44, and harvesting the arenavirus particle.

46. A method for treating a neoplastic disease in a subject in need thereof, wherein the method comprises administering to the subject an arenavirus particle, wherein
a. the arenavirus particle comprises an arenavirus genome comprising a nucleotide sequence encoding an antigenic fragment of mutant KRAS, wherein the antigenic fragment comprises the mutation; and
b. at least one arenavirus open reading frame (“ORF”) of the arenavirus genome is either (i) functionally inactivated or deleted; or (ii) located in a position other than the wild-type position of said at least one arenavirus ORF; or (iii) sequestered into two or more functional fragments and a fragment of the at least one arenavirus ORF is located in a position other than the wild-type position of said at least one arenavirus ORF.

47. The method of claim 46, wherein the mutation in KRAS is at amino acid position G12, G13, A18, A59, Q61, K117, A146, or D119 of KRAS.

48. The method of any of claims 46 to 47, wherein the mutation in KRAS is A18D, A59E, A59G, A59P, A59T, A59S, A59V, A146P, A146S, A146T, A146V, D119N, G12A, G12C, G12D, G12F, G12L, G12R, G12S, G12V, G13A, G13C, G13D, G13E, G13R, G13S, G13V, K117N, Q61E, Q61H, Q61K, Q61L, Q61P, Q61R or a combination thereof.

49. The method of any of claims 46 to 48, wherein the mutation in KRAS is G12A, G12C, G12D, G12R, G12S, G12V, G13D, Q61H, Q61R, A146T or a combination thereof.

50. The method of any of claims 46 to 49, wherein the mutation in KRAS is one, more, or all of G13D, G12V, G12C, G12D, and G12R.

51. The method of claim 50, wherein the nucleotide sequence encodes from N- to C-terminus fragments of mutant KRAS comprising the mutations G13D, G12V, G12C, G12D, and G12R, respectively.

52. The method of claim 50 wherein the mutations in KRAS are all of G13D, G12V, G12C, G12D, and G12R in any possible order.

53. The method of claim 46, wherein the arenavirus genome comprises a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of SEQ ID NO:20.

54. The method of claim 46, wherein the arenavirus genome comprises a nucleotide sequence encoding an expression product whose amino acid sequence is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:19.

55. The method of any of claims 46 to 54, wherein the antigenic fragment of mutant KRAS is 8, 9, 10, 11, 12, 13, 14, 15, 16, 17, 18, 19, 20, 21, 22, 23, 24, 25, 26, 27, 28, 29, or 30 amino acids long.

56. The method of any of claims 46 to 54, wherein the antigenic fragment of mutant KRAS is 18 amino acids long.

57. The method of any of claims 46 to 52, wherein the region flanking the mutation at the N-terminus of the antigenic fragment is 0, 1, 2, 3, 4, 5, 6, 7, 8, 9, 10, 11, 12, 13, 14, 15, 16, 17, 18, 19, or 20 amino acids long.

58. The method of any of claims 46 to 52, wherein the region flanking the mutation at the N-terminus of the antigenic fragment is 8 or 9 amino acids long.

59. The method of any of claims 46 to 58, wherein the region flanking the mutation at the C-terminus of the antigenic fragment is 0, 1, 2, 3, 4, 5, 6, 7, 8, 9, 10, 11, 12, 13, 14, 15, 16, 17, 18, 19, or 20 amino acids long.

60. The method of any of claims 46 to 58, wherein the region flanking the mutation at the C-terminus of the antigenic fragment is 8 or 9 amino acids long.

61. The method of any of claims 46 to 60, wherein the nucleotide sequence encodes two, three, four, five, six, seven, eight, nine, 10, 11, 12, 13, 14, 15, 16, 17, 18, 19, or 20 antigenic fragments of mutant KRAS, and wherein each antigenic fragment comprises the mutation of one of the mutant KRAS proteins.

62. The method of any of claims 46 to 60, wherein the nucleotide sequence encodes five antigenic fragments of mutant KRAS, and wherein each antigenic fragment comprises the mutation of one of the mutant KRAS proteins.

63. The method of claim 62, wherein the five antigenic fragments of mutant KRAS comprise the mutations G13D, G12V, G12C, G12D, and G12R.

64. The method of claim 61, wherein the antigenic fragments comprise the same or different mutations of mutant KRAS proteins.

65. The method of claim 61, wherein the antigenic fragments are fused to each other via the same or different linkers.

66. The method of claim any one of claims 61-63, wherein the antigenic fragments are fused directly to each other without intervening sequences.

67. The method of claim 65, wherein the linker is AAY linker (AAY), AAA linker (AAA), GS linker (GGSGGGGSGG) (SEQ ID NO:42), or variants of AAY, AAA, and GS linker sequences optimized via in silico prediction.

68. The method of any of claims 46 to 67, wherein the nucleotide sequence is engineered to reduce or remove any CpG and TpA islands.

69. The method of claim 68, wherein the removal of the CpG and TpA islands comprises three cycles:
(i) CpG is removed in a first cycle;
(ii) TpA is removed in a second cycle; and
(iii) CpG is removed in a third cycle to remove newly introduced CpG in the second cycle.

70. The method of any of claims 46 to 69, wherein the arenavirus genome comprises:
(i) a first S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral glycoprotein (“GP”) under control of an arenavirus genomic 3′ UTR; and
(iii) an L-Segment.

71. The method of any of claims 46 to 64, 66, and 68 to 70, wherein the arenavirus genome comprises:
(i) a first S-Segment comprising the nucleotide sequence of SEQ ID NO:20 under control of an arenavirus genomic 5′ UTR and an ORF encoding the arenaviral nucleoprotein (“NP”) under control of an arenavirus genomic 3′ UTR; and
(ii) a second S-Segment comprising the nucleotide sequence of SEQ ID NO:20 under control of an arenavirus genomic 5′ UTR and an ORF encoding the arenaviral glycoprotein (“GP”) under control of an arenavirus genomic 3′ UTR; and
(iii) an L-Segment.

72. The method of any of claims 46 to 64, 66, and 68 to 70, wherein the arenavirus genome comprises:
(i) a first S-Segment comprising the nucleotide sequence of SEQ ID NO:21; and
(ii) a second S-Segment comprising the nucleotide sequence of SEQ ID NO:22; and
(iii) an L-Segment.

73. The method of any of claims 46 to 64, 66, and 68 to 70, wherein the arenavirus genome comprises:
(i) a first S-Segment comprising the nucleotide sequence of SEQ ID NO:23; and
(ii) a second S-Segment comprising the nucleotide sequence of SEQ ID NO:24; and
(iii) an L-Segment.

74. The method of any of claims 46 to 69, wherein the arenavirus genome comprises:
(i) a first S-Segment comprising an ORF encoding the arenavirus GP1 and GP2 subunits fused to a heterologous signal peptide under control of an arenavirus genomic 5′ UTR and an ORF encoding a fusion of arenavirus GP signal peptide and a nucleotide sequence encoding the antigenic fragment(s) under control of an arenavirus genomic 3′ UTR; and
(ii) a second S-Segment comprising the nucleotide sequence encoding the antigenic fragment(s) under control of an arenavirus genomic 5′ UTR and an ORF encoding the arenaviral nucleoprotein (“NP”) under control of an arenavirus genomic 3′ UTR; and
(iii) an L-Segment.

75. The method of claims 70 and 74, wherein the nucleotide sequence encoding the antigenic fragment(s) on the first S-Segment is different from the nucleotide sequence encoding the antigenic fragment(s) on the second S-Segment.

76. The method of claims 70 and 74, wherein the nucleotide sequence encoding the antigenic fragment(s) on the first S-Segment is the same as the nucleotide sequence encoding the antigenic fragment(s) on the second S-Segment.

77. The method of claims 70 and 74, wherein the antigenic fragment(s) encoded on the first S-Segment is/are different from the antigenic fragment(s) encoded on the second S-Segment.

78. The method of claims 70 and 74, wherein the antigenic fragment(s) encoded on the first S-Segment is/are the same as the antigenic fragment(s) encoded on the second S-Segment.

79. The method of claim 78, wherein the antigenic fragments encoded on the first S-Segment are the same as the antigenic fragments encoded on the second S-Segment but are fused to each other in a different order from the order in which the antigenic fragments encoded on the second S-Segment are fused to each other.

80. The method of any of claims 46 to 79, wherein the neoplastic disease is pancreatic cancer, colorectal cancer, lung adenocarcinoma, lung squamous cell carcinoma, or non-small cell lung cancer (NSCLC).

81. The method of any of claims 46 to 79, wherein the arenavirus genome comprises a nucleotide sequence encoding antigenic fragments of mutant KRAS, wherein the antigenic fragments comprise the mutation, wherein the mutation in KRAS is G12D, G12V, G12R, Q61H, Q61R, G12C, G12S, and/or G12A and wherein the neoplastic disease is pancreatic cancer.

82. The method of any of claims 46 to 79, wherein the arenavirus genome comprises a nucleotide sequence encoding antigenic fragments of mutant KRAS, wherein the antigenic fragments comprise the mutation, and wherein the mutation in KRAS is G13D, G12V, G12C, G12D, and G12R and wherein the neoplastic disease is pancreatic cancer, colorectal cancer or lung adenocarcinoma.

83. The method of any of claims 46 to 71 and 74 to 79, wherein the arenavirus genome further comprises a nucleotide sequence encoding an antigenic fragment of mutant TP53, wherein the antigenic fragment comprises the mutation, wherein the mutation in KRAS is G12D, G12R, G12V, Q61H and/or Q61R, wherein the mutation in TP53 is R175H, R248W, G245S, R282W, R248Q and/or R273C, and wherein the neoplastic disease is pancreatic cancer.

84. The method of any of claims 46 to 71 and 74 to 79, wherein the arenavirus genome further comprises a nucleotide sequence encoding an antigenic fragment of mutant TP53, and/or mutant U2AF1, wherein the antigenic fragment comprises the mutation, wherein the mutation in KRAS is G12D, G12V, G12R, Q61H and/or Q61R, wherein the mutation in TP53 is R175H, R248W, G245S, R282W, R248Q and/or R273C, wherein the mutation in U2AF1 is S34F, and wherein the neoplastic disease is pancreatic cancer.

85. The method of any of claims 46 to 71 and 74 to 79, wherein the arenavirus genome further comprises a nucleotide sequence encoding an antigenic fragment of mutant BRAF, mutant TP53, mutant FBXW7 and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is colorectal cancer.

86. The method of any of claims 46 to 71 and 74 to 79, wherein the arenavirus genome further comprises a nucleotide sequence encoding an antigenic fragment of mutant BRAF, mutant TP53, mutant FBXW7, and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is lung adenocarcinoma.

87. The method of any of claims 46 to 71 and 74 to 79, wherein the arenavirus genome further comprises a nucleotide sequence encoding an antigenic fragment of mutant BRAF, mutant PIK3CA, mutant EGFR, mutant TP53, and/or mutant U2AF1, wherein the antigenic fragment comprises the mutation, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, wherein the mutation in U2AF1 is S34F, and wherein the neoplastic disease is pancreatic cancer, colorectal cancer or lung adenocarcinoma.

88. The method of any of claims 46 to 79, wherein the arenavirus genome comprises a nucleotide sequence encoding antigenic fragments of mutant KRAS, wherein the antigenic fragment comprises the mutation, and wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma.

89. The method of any of claims 46 to 71 and 74 to 79, wherein the arenavirus genome further comprises a nucleotide sequence encoding an antigenic fragment of mutant TP53, mutant U2AF1, mutant PIK3CA, mutant EGFR and/or mutant BRAF, wherein the antigenic fragment comprises the mutation, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E, and wherein the neoplastic disease is lung adenocarcinoma.

90. The method of any of claims 46 to 71 and 74 to 79, wherein the arenavirus genome further comprises a nucleotide sequence encoding an antigenic fragment of mutant AKT1, mutant BRAF, mutant HER2, mutant MEK1, mutant MET, mutant NRAS, mutant PIK3CA and/or mutant RET, wherein the antigenic fragment comprises the mutation, and wherein the neoplastic disease is non-small cell lung cancer (NSCLC).

91. The method of any of claims 46 to 71 and 74 to 90, wherein the arenavirus genome further comprises a nucleotide sequence encoding an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation.

92. The method of any of claims 46 to 71 and 74 to 91, wherein the arenavirus genome further comprises a nucleotide sequence encoding an antigenic fragment of mutant PI3KCA, wherein the antigenic fragment comprises the mutation, and wherein the mutation in PI3KCA is E545K, H1047R and/or E542K.

93. The method of any of claims 46 to 71 and 74 to 92, wherein the arenavirus genome further comprises a nucleotide sequence encoding an antigenic fragment of mutant BRAF, wherein the antigenic fragment comprises the mutation, and wherein the mutation in BRAF is V600E.

94. The method of any of claims 46 to 71 and 74 to 93, wherein the arenavirus genome further comprises a nucleotide sequence encoding an antigenic fragment of mutant U2AF1, wherein the antigenic fragment comprises the mutation, and wherein the mutation in U2AF1 is S34F.

95. The method of any of claims 46 to 71 and 74 to 94, wherein the arenavirus genome further comprises a nucleotide sequence encoding an antigenic fragment of mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W and/or C277F.

96. The method of any of claims 46 to 71 and 74 to 95, wherein the arenavirus genome further comprises a nucleotide sequence encoding an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family.

97. The method of any of claims 46 to 85, 87 to 88, and 91 to 96, wherein the method further comprises administering a second arenavirus particle, wherein the second arenavirus particle encodes an antigenic fragment of mutant KRAS, wherein the antigenic fragment comprises the mutation, and wherein the neoplastic disease is pancreatic cancer or colorectal cancer.

98. The method of any of claims 46 to 84, 87 to 88, and 91 to 96, wherein the method further comprises administering a second arenavirus particle, wherein the second arenavirus particle encodes an antigenic fragment of mutant KRAS, wherein the antigenic fragments comprise the mutation, wherein the mutation in KRAS is G12D, G12V, G12R, Q61H, Q61R, G12C, G12S and/or G12A and wherein the neoplastic disease is pancreatic cancer.

99. The method of any of claims 46 to 84, 87 to 88, and 91 to 96, wherein the method further comprises administering a second arenavirus particle, wherein the second arenavirus particle encodes an antigenic fragment of mutant KRAS and/or mutant TP53, wherein the antigenic fragment comprises the mutation, wherein the mutation in KRAS is G12D, G12R, G12V Q61H and/or Q61R, wherein the mutation in TP53 is R175H, R248W, G245S, R282W, R248Q and/or R273C, and wherein the neoplastic disease is pancreatic cancer.

100. The method of any of claims 46 to 84, 87 to 88, and 91 to 96, wherein the method further comprises administering a second arenavirus particle, wherein the second arenavirus particle encodes an antigenic fragment of mutant KRAS, mutant TP53, mutant U2AF1, wherein the antigenic fragment comprises the mutation, wherein the mutation in KRAS is G12D, G12V, G12R, Q61H and/or Q61R, wherein the mutation in TP53 is R175H, R248W, G245S, R282W, R248Q and/or R273C, wherein the mutation in U2AF1 is S34F, and wherein the neoplastic disease is pancreatic cancer.

101. The method of any of claims 46 to 80, 82, 85, 87 to 88 and 91 to 96, wherein the method further comprises administering a second arenavirus particle, wherein the second arenavirus particle encodes an antigenic fragment of mutant KRAS, mutant BRAF, mutant TP53, mutant FBXW7, mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H and/or R465C, wherein the mutation in PIK3CA is E545K and/or H1047R, and wherein the neoplastic disease is colorectal cancer.

102. The method of any of claims 46 to 80, 82, 86 to 89, and 91 to 96, wherein the method further comprises administering a second arenavirus particle, wherein the second arenavirus particle encodes an antigenic fragment of mutant KRAS, mutant BRAF, mutant TP53, mutant FBXW7 and/or mutant PIK3CA, wherein the antigenic fragment comprises the mutation, wherein the mutation in KRAS is G12D, G12V, G13D, A146T and/or G12C, wherein the mutation in BRAF is V600E, wherein the mutation in TP53 is R175H, R273H and/or R248W, wherein the mutation in FBXW7 is R465H, wherein the mutation in PIK3CA is E545K and/or H1047R, and wherein the neoplastic disease is lung adenocarcinoma.

103. The method of any of claims 46 to 89, and 91 to 96, wherein the method further comprises administering a second arenavirus particle, wherein the second arenavirus particle encodes an antigenic fragment of mutant KRAS, mutant BRAF, mutant PIK3CA, mutant EGFR, mutant TP53, and/or mutant U2AF1, wherein the antigenic fragment comprises the mutation, wherein the mutation in KRAS is G12C, G12D, G12R, G13D and/or G12V, wherein the mutation in BRAF is V600E, wherein the mutation in PIK3CA is E545K, wherein the mutation in EGFR is L858R, wherein the mutation in TP53 is R175H, R273H and/or R248W, wherein the mutation in U2AF1 is S34F, and wherein the neoplastic disease is pancreatic cancer, colorectal cancer or lung adenocarcinoma.

104. The method of any of claims 46 to 89, and 91 to 96, wherein the method further comprises administering a second arenavirus particle, wherein the second arenavirus particle encodes an antigenic fragment of mutant KRAS, wherein the antigenic fragment comprises the mutation, and wherein the mutation in KRAS is G12D, G12V, G12C, G12R, G13D, A146T, G12S, Q61H, G12A, and/or Q61R and wherein the neoplastic disease is pancreatic cancer, colorectal cancer or lung adenocarcinoma.

105. The method of any of claims 46 to 89, and 91 to 96, wherein the method further comprises administering a second arenavirus particle, wherein the second arenavirus particle encodes an antigenic fragment of mutant KRAS, wherein the antigenic fragment comprises the mutation, and wherein the mutation in KRAS is G13D, G12V, G12C, G12D, and G12R and wherein the neoplastic disease is pancreatic cancer, colorectal cancer or lung adenocarcinoma.

106. The method of any of claims 46 to 80, 82, 86 to 89, and 91 to 96, wherein the method further comprises administering a second arenavirus particle, wherein the second arenavirus particle encodes an antigenic fragment of mutant KRAS, mutant TP53, mutant U2AF1, mutant PIK3CA, mutant EGFR and/or mutant BRAF, wherein the antigenic fragment comprises the mutation, wherein the mutation in KRAS is G12C, G12V, G12D, and/or G12S, wherein the mutation in TP53 is R175H, wherein the mutation in U2AF1 is S34F, wherein the mutation in EGFR is L858R, L861Q and/or E746_A750del, wherein the mutation in PIK3CA is E545K and/or E542K, wherein the mutation in BRAF is V600E, and wherein the neoplastic disease is lung adenocarcinoma.

107. The method of any of claims 46 to 106, wherein the method further comprises administering a second arenavirus particle, wherein the second arenavirus particle encodes an antigenic fragment of mutant TP53, and wherein the antigenic fragment comprises the mutation.

108. The method of any of claims 46 to 106, wherein the method further comprises administering a second arenavirus particle, wherein the second arenavirus particle encodes an antigenic fragment of mutant AKT1, mutant BRAF, mutant HER2, mutant MEK1, mutant MET, mutant NRAS, mutant PIK3CA, mutant RET, mutant APC, mutant U2AF1, mutant EGFR, mutant FBXW7, mutant SMAD4, mutant GNAS, mutant ERBB2, mutant ERBB3, mutant CDKN2A, mutant TP53 and/or mutant CTNNB1, and wherein the antigenic fragment comprises the respective mutation.

109. The method of any of claims 46 to 96, wherein the method further comprises administering a second arenavirus particle, wherein the second arenavirus particle encodes an antigenic fragment of mutant PI3KCA, wherein the antigenic fragment comprises the mutation, and wherein the mutation in PI3KCA is E545K, H1047R and/or E542K.

110. The method of any of claims 46 to 96, wherein the method further comprises administering a second arenavirus particle, wherein the second arenavirus particle encodes an antigenic fragment of mutant BRAF, wherein the antigenic fragment comprises the mutation, and wherein the mutation in BRAF is V600E.

111. The method of any of claims 46 to 96, wherein the method further comprises administering a second arenavirus particle, wherein the second arenavirus particle encodes an antigenic fragment of mutant U2AF1, wherein the antigenic fragment comprises the mutation, and wherein the mutation in U2AF1 is S34F.

112. The method of any of claims 46 to 96, wherein the method further comprises administering a second arenavirus particle, wherein the second arenavirus particle encodes an antigenic fragment of mutant TP53, wherein the antigenic fragment comprises the mutation, and wherein the mutation in TP53 is G245S, Y220C, R248Q, R282W, H179R, V157F, R273C, R213L, R273H, R273L, R175H, R158L, R196P, R248W and/or C277F.

113. The method of any of claims 46 to 96, wherein the method further comprises administering a second arenavirus particle, wherein the second arenavirus particle encodes an antigenic fragment of BIRC family, CEACAM family, CTA family, EPH family, ERBB family, FOLR family, GAST family, GUCY2 family, IDO family, IL13RA family, KDR family, KLK family, MAGE family, MUC family, PEMT family, SDC family, SLAMF family, TERT family, TLR family, TPTE family, TYR family, WT family and/or XBP family.

114. The method of any of claims 46 to 96, wherein the method further comprises administering a second arenavirus particle, wherein the second arenavirus particle comprises an arenavirus genome comprising the nucleotide sequences of SEQ ID NOs:21 and 22.

115. The method of any of claims 46 to 96, wherein the method further comprises administering a second arenavirus particle, wherein the second arenavirus particle comprises an arenavirus genome comprising the nucleotide sequences of SEQ ID NOs:23 and 24.

116. The method of any of claims 46 to 115, wherein the arenavirus particle is derived from lymphocytic choriomeningitis virus (LCMV) or Pichinde virus.

117. The method of any of claims 46 to 116, wherein the neoplastic disease is a solid tumor, and wherein the method results in an increase of the concentration of T cells within the solid tumor.

118. The method of any of claims 46 to 79, 91 to 96, and 107 to 117, wherein the neoplastic disease is acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; childhood thyroid cancer; transitional cell cancer of the renal pelvis and ureter; urethral cancer; uterine cancer, endometrial; uterine sarcoma; vaginal cancer; vulvar cancer; and Wilms tumor.

119. The method of any of claims 46 to 117, wherein the neoplastic disease is a solid tumor, and wherein the route of administration of the arenavirus particle is via intratumoral injection.


SEQUENCE LISTING (42 sequences)

SEQ ID NO: 1 - DNA, 354 bp, synthetic construct
  source 1..354: mol_type = other DNA; note = Nucleotide sequence of 4xKRASmut
atgacagagtacaaactggtggtggtgggtgctgtgggggtgggaaaaagtgccttgaccattcagctcattcagaatcactttgtggacacagagtacaagttggtggttgtcggagcaggtgatgttggaaaatcagccctcaccatccagctgatccagaaccactttgttgacacagaatacaagctggtggttgtgggggctgatggagtgggcaagagtgcactcaccatccaactgatccagaatcattttgtggacactgagtacaagctggttgtggtgggggcctgtggggtgggcaaatcagccctcaccattcaactcattcaaaaccattttgtggattga

SEQ ID NO: 2 - DNA, 441 bp, synthetic construct
  source 1..441: mol_type = other DNA; note = Nucleotide sequence of 4xKRASmut_E7
atgacagagtacaaactggtggtggtgggtgctgtgggggtgggaaaaagtgccttgaccattcagctcattcagaatcactttgtggacacagagtacaagttggtggttgtcggagcaggtgatgttggaaaatcagccctcaccatccagctgatccagaaccactttgttgacacagaatacaagctggtggttgtgggggctgatggagtgggcaagagtgcactcaccatccaactgatccagaatcattttgtggacactgagtacaagctggttgtggtgggggcctgtggggtgggcaaatcagccctcaccattcaactcattcaaaaccattttgtggatatgcatggggacacccccaccctgcatgagtacatgctggacctccagcctgagaccacagacctgtactgctatgagcagctgaactga

SEQ ID NO: 3 - DNA, 441 bp, synthetic construct
  source 1..441: mol_type = other DNA; note = Nucleotide sequence of 4xKRASmut_EBV
atgacagagtacaaactggtggtggtgggtgctgtgggggtgggaaaaagtgccttgaccattcagctcattcagaatcactttgtggacacagagtacaagttggtggttgtcggagcaggtgatgttggaaaatcagccctcaccatccagctgatccagaaccactttgttgacacagaatacaagctggtggttgtgggggctgatggagtgggcaagagtgcactcaccatccaactgatccagaatcattttgtggacactgagtacaagctggttgtggtgggggcctgtggggtgggcaaatcagccctcaccattcaactcattcaaaaccattttgtggatgatgccaagagcaccaagaagtgcagagccattgtgactgacttcagtgtgatcaaggccatagaggaggagcacagaaagaagaagtga

SEQ ID NO: 4 - protein, 117 aa, synthetic construct
  source 1..117: note = Amino acid sequence of 4xKRASmut
MTEYKLVVVGAVGVGKSALTIQLIQNHFVDTEYKLVVVGAGDVGKSALTIQLIQNHFVDTEYKLVVVGADGVGKSALTIQLIQNHFVDTEYKLVVVGACGVGKSALTIQLIQNHFVD

SEQ ID NO: 5 - protein, 146 aa, synthetic construct
  source 1..146: note = Amino acid sequence of 4xKRASmut_E7
MTEYKLVVVGAVGVGKSALTIQLIQNHFVDTEYKLVVVGAGDVGKSALTIQLIQNHFVDTEYKLVVVGADGVGKSALTIQLIQNHFVDTEYKLVVVGACGVGKSALTIQLIQNHFVDMHGDTPTLHEYMLDLQPETTDLYCYEQLN

SEQ ID NO: 6 - protein, 146 aa, synthetic construct
  source 1..146: note = Amino acid sequence of 4xKRASmut_EBV
MTEYKLVVVGAVGVGKSALTIQLIQNHFVDTEYKLVVVGAGDVGKSALTIQLIQNHFVDTEYKLVVVGADGVGKSALTIQLIQNHFVDTEYKLVVVGACGVGKSALTIQLIQNHFVDDAKSTKKCRAIVTDFSVIKAIEEEHRKKK

SEQ ID NO: 7 - DNA, 1224 bp, synthetic construct
  source 1..1224: mol_type = other DNA; note = Nucleotide sequence of 14xp53 (LCMV backbone)
atgacgatacactacaattatatgtgcaacagcagctgtatgggctcgatgaacagaagacccatcctgacaatcattaccctggaggatttggatgaccgcaatactttccgacactcagtcgtagtgccgtgtgaaccccccgaggtagggagcgactgcaccactattcattattataattacatgtgtaacagcagttgcatgggtggtatgaaccaacggcccatcctgactatcatcacccttgaagattcaagcgggaacagctttgaggtaagagtgtgtgcctgcccgggaagagactggcgcacggaagaagagaatctgagaaagaagggcgagccccaccagtcacaacatatgacagaagtagtgagaagatgccctagaagagagagatgcagtgattccgatggcctggcgccaccacaacatgtacagttatgggtagactccacgcctccccccggcacaagattcagagccatggcgatctacaaacagtcgcaacacatgactgaggattcaagcggtaaccttttgggccggaatagctttgaagtgtgcgtttgcgcgtgccccggcagagatagacggaccgaggaagagggtaatctgagagtggaatatctggatgataggaataccttcctccacagcgtcgtggtcccctacgaaccccccgaggtggggtcagactcctctggcaacttgctgggtcggaacagcttcgaagtgcacgtgtgcgcatgtcctggcagagatcgccgcacagaggaggaggactcgagcgggaacctgctcggtcggaacagtttcgaggtgctggtctgcgcatgtcccggcagagaccgcagaaccgaggaggaggcgatttataagcaatcgcaacatatgacggaggtagtgcgacactgcccccaccacgagcggtgtagcgatagtgatgggctagctcagctgtgggtggatagcaccccaccgcccggcacccgcgttctggcgatggctatctataaacaatctcagcatatgactgaagtgtgctccgactcggacggacttgccccccctcagcacttaatccctgtggaggggaacctgagggtggagtatttggatgatcgcaattacaactatatgtgtaattctagttgtatgggtggaatgaactggaggcccattctaaccattatcacactcgaagattctagcggctga

SEQ ID NO: 8 - DNA, 1311 bp, synthetic construct
  source 1..1311: mol_type = other DNA; note = Nucleotide sequence of 14xp53_E7 (LCMV backbone)
atgacgatacactacaattatatgtgcaacagcagctgtatgggctcgatgaacagaagacccatcctgacaatcattaccctggaggatttggatgaccgcaatactttccgacactcagtcgtagtgccgtgtgaaccccccgaggtagggagcgactgcaccactattcattattataattacatgtgtaacagcagttgcatgggtggtatgaaccaacggcccatcctgactatcatcacccttgaagattcaagcgggaacagctttgaggtaagagtgtgtgcctgcccgggaagagactggcgcacggaagaagagaatctgagaaagaagggcgagccccaccagtcacaacatatgacagaagtagtgagaagatgccctagaagagagagatgcagtgattccgatggcctggcgccaccacaacatgtacagttatgggtagactccacgcctccccccggcacaagattcagagccatggcgatctacaaacagtcgcaacacatgactgaggattcaagcggtaaccttttgggccggaatagctttgaagtgtgcgtttgcgcgtgccccggcagagatagacggaccgaggaagagggtaatctgagagtggaatatctggatgataggaataccttcctccacagcgtcgtggtcccctacgaaccccccgaggtggggtcagactcctctggcaacttgctgggtcggaacagcttcgaagtgcacgtgtgcgcatgtcctggcagagatcgccgcacagaggaggaggactcgagcgggaacctgctcggtcggaacagtttcgaggtgctggtctgcgcatgtcccggcagagaccgcagaaccgaggaggaggcgatttataagcaatcgcaacatatgacggaggtagtgcgacactgcccccaccacgagcggtgtagcgatagtgatgggctagctcagctgtgggtggatagcaccccaccgcccggcacccgcgttctggcgatggctatctataaacaatctcagcatatgactgaagtgtgctccgactcggacggacttgccccccctcagcacttaatccctgtggaggggaacctgagggtggagtatttggatgatcgcaattacaactatatgtgtaattctagttgtatgggtggaatgaactggaggcccattctaaccattatcacactcgaagattctagcggcatgcatggggacacccccaccctgcatgagtacatgctggacctccagcctgagaccacagacctgtactgctatgagcagctgaactga

SEQ ID NO: 9 - DNA, 1311 bp, synthetic construct
  source 1..1311: mol_type = other DNA; note = Nucleotide sequence of 14xp53_EBV (LCMV backbone)
atgacgatacactacaattatatgtgcaacagcagctgtatgggctcgatgaacagaagacccatcctgacaatcattaccctggaggatttggatgaccgcaatactttccgacactcagtcgtagtgccgtgtgaaccccccgaggtagggagcgactgcaccactattcattattataattacatgtgtaacagcagttgcatgggtggtatgaaccaacggcccatcctgactatcatcacccttgaagattcaagcgggaacagctttgaggtaagagtgtgtgcctgcccgggaagagactggcgcacggaagaagagaatctgagaaagaagggcgagccccaccagtcacaacatatgacagaagtagtgagaagatgccctagaagagagagatgcagtgattccgatggcctggcgccaccacaacatgtacagttatgggtagactccacgcctccccccggcacaagattcagagccatggcgatctacaaacagtcgcaacacatgactgaggattcaagcggtaaccttttgggccggaatagctttgaagtgtgcgtttgcgcgtgccccggcagagatagacggaccgaggaagagggtaatctgagagtggaatatctggatgataggaataccttcctccacagcgtcgtggtcccctacgaaccccccgaggtggggtcagactcctctggcaacttgctgggtcggaacagcttcgaagtgcacgtgtgcgcatgtcctggcagagatcgccgcacagaggaggaggactcgagcgggaacctgctcggtcggaacagtttcgaggtgctggtctgcgcatgtcccggcagagaccgcagaaccgaggaggaggcgatttataagcaatcgcaacatatgacggaggtagtgcgacactgcccccaccacgagcggtgtagcgatagtgatgggctagctcagctgtgggtggatagcaccccaccgcccggcacccgcgttctggcgatggctatctataaacaatctcagcatatgactgaagtgtgctccgactcggacggacttgccccccctcagcacttaatccctgtggaggggaacctgagggtggagtatttggatgatcgcaattacaactatatgtgtaattctagttgtatgggtggaatgaactggaggcccattctaaccattatcacactcgaagattctagcggcgatgccaagagcaccaagaagtgcagagccattgtgactgacttcagtgtgatcaaggccatagaggaggagcacagaaagaagaagtga

SEQ ID NO: 10 - DNA, 1224 bp, synthetic construct
  source 1..1224: mol_type = other DNA; note = Nucleotide sequence of 14xp53 (PICV backbone)
atgttggatgaccgcaatactttccgacactcagtcgtagtgccgtgtgaaccccccgaggtagggagcgactgcaccactattcattattacaactatatgtgtaattctagttgtatgggtggaatgaactggaggcccattctaaccattatcacactcgaagattctagcggcgactcctctggcaacttgctgggtcggaacagcttcgaagtgcacgtgtgcgcatgtcctggcagagatcgccgcacagaggaggagacgatacactacaattatatgtgcaacagcagctgtatgggctcgatgaacagaagacccatcctgacaatcattaccctggaggatgactcgagcgggaacctgctcggtcggaacagtttcgaggtgctggtctgcgcatgtcccggcagagaccgcagaaccgaggaggagtgctccgactcggacggacttgccccccctcagcacttaatccctgtggaggggaacctgagggtggagtatttggatgatcgcaatgtacagttatgggtagactccacgcctccccccggcacaagattcagagccatggcgatctacaaacagtcgcaacacatgactgaggattcaagcggtaaccttttgggccggaatagctttgaagtgtgcgtttgcgcgtgccccggcagagatagacggaccgaggaagagcagctgtgggtggatagcaccccaccgcccggcacccgcgttctggcgatggctatctataaacaatctcagcatatgactgaagtgaacagctttgaggtaagagtgtgtgcctgcccgggaagagactggcgcacggaagaagagaatctgagaaagaagggcgagccccaccagtcacaacatatgacagaagtagtgagaagatgccctagaagagagagatgcagtgattccgatggcctggcgccaccacaacatggtaatctgagagtggaatatctggatgataggaataccttcctccacagcgtcgtggtcccctacgaaccccccgaggtggggtcagcgatttataagcaatcgcaacatatgacggaggtagtgcgacactgcccccaccacgagcggtgtagcgatagtgatgggctagcttataattacatgtgtaacagcagttgcatgggtggtatgaaccaacggcccatcctgactatcatcacccttgaagattcaagcgggtga

SEQ ID NO: 11 - DNA, 1311 bp, synthetic construct
  source 1..1311: mol_type = other DNA; note = Nucleotide sequence of 14xp53_E7 (PICV backbone)
atgttggatgaccgcaatactttccgacactcagtcgtagtgccgtgtgaaccccccgaggtagggagcgactgcaccactattcattattacaactatatgtgtaattctagttgtatgggtggaatgaactggaggcccattctaaccattatcacactcgaagattctagcggcgactcctctggcaacttgctgggtcggaacagcttcgaagtgcacgtgtgcgcatgtcctggcagagatcgccgcacagaggaggagacgatacactacaattatatgtgcaacagcagctgtatgggctcgatgaacagaagacccatcctgacaatcattaccctggaggatgactcgagcgggaacctgctcggtcggaacagtttcgaggtgctggtctgcgcatgtcccggcagagaccgcagaaccgaggaggagtgctccgactcggacggacttgccccccctcagcacttaatccctgtggaggggaacctgagggtggagtatttggatgatcgcaatgtacagttatgggtagactccacgcctccccccggcacaagattcagagccatggcgatctacaaacagtcgcaacacatgactgaggattcaagcggtaaccttttgggccggaatagctttgaagtgtgcgtttgcgcgtgccccggcagagatagacggaccgaggaagagcagctgtgggtggatagcaccccaccgcccggcacccgcgttctggcgatggctatctataaacaatctcagcatatgactgaagtgaacagctttgaggtaagagtgtgtgcctgcccgggaagagactggcgcacggaagaagagaatctgagaaagaagggcgagccccaccagtcacaacatatgacagaagtagtgagaagatgccctagaagagagagatgcagtgattccgatggcctggcgccaccacaacatggtaatctgagagtggaatatctggatgataggaataccttcctccacagcgtcgtggtcccctacgaaccccccgaggtggggtcagcgatttataagcaatcgcaacatatgacggaggtagtgcgacactgcccccaccacgagcggtgtagcgatagtgatgggctagcttataattacatgtgtaacagcagttgcatgggtggtatgaaccaacggcccatcctgactatcatcacccttgaagattcaagcgggatgcatggggacacccccaccctgcatgagtacatgctggacctccagcctgagaccacagacctgtactgctatgagcagctgaactga

SEQ ID NO: 12 - DNA, 1311 bp, synthetic construct
  source 1..1311: mol_type = other DNA; note = Nucleotide sequence of 14xp53_EBV (PICV backbone)
atgttggatgaccgcaatactttccgacactcagtcgtagtgccgtgtgaaccccccgaggtagggagcgactgcaccactattcattattacaactatatgtgtaattctagttgtatgggtggaatgaactggaggcccattctaaccattatcacactcgaagattctagcggcgactcctctggcaacttgctgggtcggaacagcttcgaagtgcacgtgtgcgcatgtcctggcagagatcgccgcacagaggaggagacgatacactacaattatatgtgcaacagcagctgtatgggctcgatgaacagaagacccatcctgacaatcattaccctggaggatgactcgagcgggaacctgctcggtcggaacagtttcgaggtgctggtctgcgcatgtcccggcagagaccgcagaaccgaggaggagtgctccgactcggacggacttgccccccctcagcacttaatccctgtggaggggaacctgagggtggagtatttggatgatcgcaatgtacagttatgggtagactccacgcctccccccggcacaagattcagagccatggcgatctacaaacagtcgcaacacatgactgaggattcaagcggtaaccttttgggccggaatagctttgaagtgtgcgtttgcgcgtgccccggcagagatagacggaccgaggaagagcagctgtgggtggatagcaccccaccgcccggcacccgcgttctggcgatggctatctataaacaatctcagcatatgactgaagtgaacagctttgaggtaagagtgtgtgcctgcccgggaagagactggcgcacggaagaagagaatctgagaaagaagggcgagccccaccagtcacaacatatgacagaagtagtgagaagatgccctagaagagagagatgcagtgattccgatggcctggcgccaccacaacatggtaatctgagagtggaatatctggatgataggaataccttcctccacagcgtcgtggtcccctacgaaccccccgaggtggggtcagcgatttataagcaatcgcaacatatgacggaggtagtgcgacactgcccccaccacgagcggtgtagcgatagtgatgggctagcttataattacatgtgtaacagcagttgcatgggtggtatgaaccaacggcccatcctgactatcatcacccttgaagattcaagcggggatgccaagagcaccaagaagtgcagagccattgtgactgacttcagtgtgatcaaggccatagaggaggagcacagaaagaagaagtga

SEQ ID NO: 13 - protein, 407 aa, synthetic construct
  source 1..407: note = Amino acid sequence of 14xp53 (LCMV backbone)
MTIHYNYMCNSSCMGSMNRRPILTIITLEDLDDRNTFRHSVVVPCEPPEVGSDCTTIHYYNYMCNSSCMGGMNQRPILTIITLEDSSGNSFEVRVCACPGRDWRTEEENLRKKGEPHQSQHMTEVVRRCPRRERCSDSDGLAPPQHVQLWVDSTPPPGTRFRAMAIYKQSQHMTEDSSGNLLGRNSFEVCVCACPGRDRRTEEEGNLRVEYLDDRNTFLHSVVVPYEPPEVGSDSSGNLLGRNSFEVHVCACPGRDRRTEEEDSSGNLLGRNSFEVLVCACPGRDRRTEEEAIYKQSQHMTEVVRHCPHHERCSDSDGLAQLWVDSTPPPGTRVLAMAIYKQSQHMTEVCSDSDGLAPPQHLIPVEGNLRVEYLDDRNYNYMCNSSCMGGMNWRPILTIITLEDSSG

SEQ ID NO: 14 - protein, 436 aa, synthetic construct
  source 1..436: note = Amino acid sequence of 14xp53_E7 (LCMV backbone)
MTIHYNYMCNSSCMGSMNRRPILTIITLEDLDDRNTFRHSVVVPCEPPEVGSDCTTIHYYNYMCNSSCMGGMNQRPILTIITLEDSSGNSFEVRVCACPGRDWRTEEENLRKKGEPHQSQHMTEVVRRCPRRERCSDSDGLAPPQHVQLWVDSTPPPGTRFRAMAIYKQSQHMTEDSSGNLLGRNSFEVCVCACPGRDRRTEEEGNLRVEYLDDRNTFLHSVVVPYEPPEVGSDSSGNLLGRNSFEVHVCACPGRDRRTEEEDSSGNLLGRNSFEVLVCACPGRDRRTEEEAIYKQSQHMTEVVRHCPHHERCSDSDGLAQLWVDSTPPPGTRVLAMAIYKQSQHMTEVCSDSDGLAPPQHLIPVEGNLRVEYLDDRNYNYMCNSSCMGGMNWRPILTIITLEDSSGMHGDTPTLHEYMLDLQPETTDLYCYEQLN

SEQ ID NO: 15 - protein, 436 aa, synthetic construct
  source 1..436: note = Amino acid sequence of 14xp53_EBV (LCMV backbone)
MTIHYNYMCNSSCMGSMNRRPILTIITLEDLDDRNTFRHSVVVPCEPPEVGSDCTTIHYYNYMCNSSCMGGMNQRPILTIITLEDSSGNSFEVRVCACPGRDWRTEEENLRKKGEPHQSQHMTEVVRRCPRRERCSDSDGLAPPQHVQLWVDSTPPPGTRFRAMAIYKQSQHMTEDSSGNLLGRNSFEVCVCACPGRDRRTEEEGNLRVEYLDDRNTFLHSVVVPYEPPEVGSDSSGNLLGRNSFEVHVCACPGRDRRTEEEDSSGNLLGRNSFEVLVCACPGRDRRTEEEAIYKQSQHMTEVVRHCPHHERCSDSDGLAQLWVDSTPPPGTRVLAMAIYKQSQHMTEVCSDSDGLAPPQHLIPVEGNLRVEYLDDRNYNYMCNSSCMGGMNWRPILTIITLEDSSGDAKSTKKCRAIVTDFSVIKAIEEEHRKKK

SEQ ID NO: 16 - protein, 407 aa, synthetic construct
  source 1..407: note = Amino acid sequence of 14xp53 (PICV backbone)
MLDDRNTFRHSVVVPCEPPEVGSDCTTIHYYNYMCNSSCMGGMNWRPILTIITLEDSSGDSSGNLLGRNSFEVHVCACPGRDRRTEEETIHYNYMCNSSCMGSMNRRPILTIITLEDDSSGNLLGRNSFEVLVCACPGRDRRTEEECSDSDGLAPPQHLIPVEGNLRVEYLDDRNVQLWVDSTPPPGTRFRAMAIYKQSQHMTEDSSGNLLGRNSFEVCVCACPGRDRRTEEEQLWVDSTPPPGTRVLAMAIYKQSQHMTEVNSFEVRVCACPGRDWRTEEENLRKKGEPHQSQHMTEVVRRCPRRERCSDSDGLAPPQHGNLRVEYLDDRNTFLHSVVVPYEPPEVGSAIYKQSQHMTEVVRHCPHHERCSDSDGLAYNYMCNSSCMGGMNQRPILTIITLEDSSG

SEQ ID NO: 17 - protein, 436 aa, synthetic construct
  source 1..436: note = Amino acid sequence of 14xp53_E7 (PICV backbone)
MLDDRNTFRHSVVVPCEPPEVGSDCTTIHYYNYMCNSSCMGGMNWRPILTIITLEDSSGDSSGNLLGRNSFEVHVCACPGRDRRTEEETIHYNYMCNSSCMGSMNRRPILTIITLEDDSSGNLLGRNSFEVLVCACPGRDRRTEEECSDSDGLAPPQHLIPVEGNLRVEYLDDRNVQLWVDSTPPPGTRFRAMAIYKQSQHMTEDSSGNLLGRNSFEVCVCACPGRDRRTEEEQLWVDSTPPPGTRVLAMAIYKQSQHMTEVNSFEVRVCACPGRDWRTEEENLRKKGEPHQSQHMTEVVRRCPRRERCSDSDGLAPPQHGNLRVEYLDDRNTFLHSVVVPYEPPEVGSAIYKQSQHMTEVVRHCPHHERCSDSDGLAYNYMCNSSCMGGMNQRPILTIITLEDSSGMHGDTPTLHEYMLDLQPETTDLYCYEQLN

SEQ ID NO: 18 - protein, 436 aa, synthetic construct
  source 1..436: note = Amino acid sequence of 14xp53_EBV (PICV backbone)
MLDDRNTFRHSVVVPCEPPEVGSDCTTIHYYNYMCNSSCMGGMNWRPILTIITLEDSSGDSSGNLLGRNSFEVHVCACPGRDRRTEEETIHYNYMCNSSCMGSMNRRPILTIITLEDDSSGNLLGRNSFEVLVCACPGRDRRTEEECSDSDGLAPPQHLIPVEGNLRVEYLDDRNVQLWVDSTPPPGTRFRAMAIYKQSQHMTEDSSGNLLGRNSFEVCVCACPGRDRRTEEEQLWVDSTPPPGTRVLAMAIYKQSQHMTEVNSFEVRVCACPGRDWRTEEENLRKKGEPHQSQHMTEVVRRCPRRERCSDSDGLAPPQHGNLRVEYLDDRNTFLHSVVVPYEPPEVGSAIYKQSQHMTEVVRHCPHHERCSDSDGLAYNYMCNSSCMGGMNQRPILTIITLEDSSGDAKSTKKCRAIVTDFSVIKAIEEEHRKKK

SEQ ID NO: 19 - protein, 90 aa, synthetic construct
  source 1..90: note = Amino acid sequence of 5xKRASmut-H2
YKLVVVGAGDVGKSALTIYKLVVVGAVGVGKSALTIYKLVVVGACGVGKSALTIYKLVVVGADGVGKSALTIYKLVVVGARGVGKSALTI

SEQ ID NO: 20 - DNA, 276 bp, synthetic construct
  source 1..276: mol_type = other DNA; note = Nucleotide sequence of 5xKRASmut-H2
atgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctga

SEQ ID NO: 21 - DNA, 2156 bp, synthetic construct
  source 1..2156: mol_type = other DNA; note = Nucleotide sequence of artLCMV-5xKRASmut-H2-NP-S-segment
gcgcaccggggatcctaggctttttggattgcgctttcctctagatcaactgggtgtcaggccctatcctacagaaggatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 22 - DNA, 1976 bp, synthetic construct
  source 1..1976: mol_type = other DNA; note = Nucleotide sequence of artLCMV-5xKRASmut-H2-GP-S-segment
gcgcaccggggatcctaggctttttggattgcgctttcctctagatcaactgggtgtcaggccctatcctacagaaggatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 23 - DNA, 2171 bp, synthetic construct
  source 1..2171: mol_type = other DNA; note = Nucleotide sequence of artPICV-5xKRASmut-H2-NP-S-segment
gcgcaccggggatcctaggcataccttggacgcgcatattacttgatcaaagatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 24 - DNA, 2012 bp, synthetic construct
  source 1..2012: mol_type = other DNA; note = Nucleotide sequence of artPCIV-5xKRASmut-H2-GP-S-segment
gcgcaccggggatcctaggcataccttggacgcgcatattacttgatcaaagatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 25 - protein, 130 aa, synthetic construct
  source 1..130: note = Amino acid sequence of 5xKRASmut-H1
YKLVVVGAVGVGKSALTIGGSGGGGSGGYKLVVVGARGVGKSALTIGGSGGGGSGGYKLVVVGACGVGKSALTIGGSGGGGSGGYKLVVVGAGDVGKSALTIGGSGGGGSGGYKLVVVGADGVGKSALTI

SEQ ID NO: 26 - DNA, 396 bp, synthetic construct
  source 1..396: mol_type = other DNA; note = Nucleotide sequence of 5xKRASmut-H1
atgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctga

SEQ ID NO: 27 - DNA, 2276 bp, synthetic construct
  source 1..2276: mol_type = other DNA; note = Nucleotide sequence of artLCMV-5xKRASmut-H1-NP-S-segment
gcgcaccggggatcctaggctttttggattgcgctttcctctagatcaactgggtgtcaggccctatcctacagaaggatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 28 - DNA, 2096 bp, synthetic construct
  source 1..2096: mol_type = other DNA; note = Nucleotide sequence of artLCMV-5xKRASmut-H1-GP-S-segment
gcgcaccggggatcctaggctttttggattgcgctttcctctagatcaactgggtgtcaggccctatcctacagaaggatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 29 - DNA, 2291 bp, synthetic construct
  source 1..2291: mol_type = other DNA; note = Nucleotide sequence of artPICV-5xKRASmut-H1-NP-S-segment
gcgcaccggggatcctaggcataccttggacgcgcatattacttgatcaaagatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 30 - DNA, 2132 bp, synthetic construct
  source 1..2132: mol_type = other DNA; note = Nucleotide sequence artPCIV-5xKRASmut-H1-GP-S-segment
gcgcaccggggatcctaggcataccttggacgcgcatattacttgatcaaagatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 31 - protein, 72 aa, synthetic construct
  source 1..72: note = Amino acid sequence of 4xKRASmut(18-mer)
YKLVVVGAVGVGKSALTIYKLVVVGAGDVGKSALTIYKLVVVGADGVGKSALTIYKLVVVGACGVGKSALTI

SEQ ID NO: 32 - DNA, 222 bp, synthetic construct
  source 1..222: mol_type = other DNA; note = Nucleotide sequence of 4xKRASmut(18-mer)
atgtacaagctggtggtggtgggagctgtgggagtgggaaagtctgccctcaccatctacaagctggtggtagttggagctggagatgttggcaaatcagctctcaccatctacaaactggttgtagtgggggcagatggggtgggcaaaagtgccctcaccatttacaagctggtggtggttggagcctgtggagtgggcaaaagtgccctcaccatctaa

SEQ ID NO: 33 - DNA, 60 bp, synthetic construct
  source 1..60: mol_type = other DNA; note = Nucleotide sequence KRASwt
atgtacaagctggtggtggtgggagctggaggagtgggcaagagtgccctgaccatctaa

SEQ ID NO: 34 - RNA, 2156 nt, Synthetic Construct
  source 1..2156: mol_type = other RNA; note = Nucleotide sequence of artLCMV-5xKRASmut-H2-NP-S-segment (RNA sequence of SEQ ID NO:21)
gcgcaccggggatcctaggctttttggattgcgctttcctctagatcaactgggtgtcaggccctatcctacagaaggatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 35 - RNA, 1976 nt, Synthetic Construct
  source 1..1976: mol_type = other RNA; note = Nucleotide sequence of artLCMV-5xKRASmut-H2-GP-S-segment (RNA sequence of SEQ ID NO:22)
gcgcaccggggatcctaggctttttggattgcgctttcctctagatcaactgggtgtcaggccctatcctacagaaggatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 36 - RNA, 2171 nt, synthetic construct
  source 1..2171: mol_type = other RNA; note = Nucleotide sequence of artPICV-5xKRASmut-H2-NP-S-segment (RNA sequence of SEQ ID NO:23)
gcgcaccggggatcctaggcataccttggacgcgcatattacttgatcaaagatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 37 - RNA, 2012 nt, Synthetic Construct
  source 1..2012: mol_type = other RNA; note = Nucleotide sequence of artPCIV-5xKRASmut-H2-GP-S-segment (RNA sequence of SEQ ID NO:24)
gcgcaccggggatcctaggcataccttggacgcgcatattacttgatcaaagatgtacaaacttgttgtggtgggagctggggatgtgggaaagtcagccctcacaatctacaaattggtggttgtgggtgcagttggtgttggcaaatcagccctcaccatctacaaacttgttgtagttggtgcctgtggagtagggaaatcagccctcaccatctacaagctggttgtggttggtgctgatggagtgggaaagtcagccctcaccatctacaagttggtggttgtaggtgccagaggggtgggaaaatctgccctgaccatctgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 38 - RNA, 2276 nt, Synthetic Construct
  source 1..2276: mol_type = other RNA; note = Nucleotide sequence of artLCMV-5xKRASmut-H1-NP-S-segment (RNA sequence of SEQ ID NO:27)
gcgcaccggggatcctaggctttttggattgcgctttcctctagatcaactgggtgtcaggccctatcctacagaaggatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 39 - RNA, 2096 nt, Synthetic Construct
  source 1..2096: mol_type = other RNA; note = Nucleotide sequence of artLCMV-5xKRASmut-H1-GP-S-segment (RNA sequence of SEQ ID NO:28)
gcgcaccggggatcctaggctttttggattgcgctttcctctagatcaactgggtgtcaggccctatcctacagaaggatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 40 - RNA, 2291 nt, Synthetic Construct
  source 1..2291: mol_type = other RNA; note = Nucleotide sequence of artPICV-5xKRASmut-H1-NP-S-segment (RNA sequence of SEQ ID NO:29)
gcgcaccggggatcctaggcataccttggacgcgcatattacttgatcaaagatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 41 - RNA, 2132 nt, Synthetic Construct
  source 1..2132: mol_type = other RNA; note = Nucleotide sequence artPCIV-5xKRASmut-H1-GP-S-segment (RNA sequence of SEQ ID NO:30)
gcgcaccggggatcctaggcataccttggacgcgcatattacttgatcaaagatgtacaagctggtggtagttggagcagtgggagtgggcaaatcagccttgaccattggaggctcaggtggtggaggctcaggaggctacaaacttgtggtggttggagccagaggtgtaggcaaaagtgcactcaccataggaggaagtggaggcggaggtagtggaggctacaaacttgttgttgttggggcctgtggggtgggaaagtcagcactcaccattggtggcagtggaggtggaggcagtggtggctacaagttggtggtcgtaggtgctggggatgtgggcaaatctgccctcacaattggaggctcaggaggtggaggcagtggtggctacaaattggttgtggtgggtgcagatggtgtgggcaaaagtgctttgaccatctgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 42 - protein, 10 aa, Synthetic Construct
  source 1..10: note = GS linker
GGSGGGGSGG